(19) United States
(12) Patent Application Publication
(10) Pub. No.: US 2017/0174669 A1
(43) Pub. Date: Jun. 22, 2017

(54) POCHOXIME CONJUGATES USEFUL FOR THE TREATMENT OF HSP90 RELATED PATHOLOGIES

(71) Applicants: Universite de Strasbourg, Strasbourg (FR); Le Centre National De La Recherche Scientifique, Paris (FR)

(72) Inventors: Nicolas WINSSINGER, Strasbourg (FR); Sofia BARLUENGA, Strasbourg (FR)

(21) Appl. No.: 15/250,742

(22) Filed: Aug. 29, 2016

Related U.S. Application Data
(63) Continuation of application No. 14/611,978, filed on Feb. 2, 2015, now abandoned.
Application No. 14/611,978 is a continuation of application No. 13/880,878, filed on Oct. 18, 2013, now abandoned.
(60) Provisional application No. 61/405,882, filed on Oct. 22, 2010.

(51) Int. Cl. C07D 405/12; C07D 495/04; C07D 405/14

(52) U.S. Cl. CPC C07D 405/12; C07D 405/14; C07D 495/04

(57) ABSTRACT

The present invention includes novel derivatives, analogs, and intermediates of the natural products radicicol, pochonins, pochoximes, and their syntheses. The present invention also provides a pharmaceutical composition comprising the present compound and the use of the compound as inhibitors of kinases and of the enzyme family known as heat shock protein 90 (HSP90).

### CROSS-REFERENCE TO RELATED APPLICATION

This application claims the benefit of priority to U.S. Provisional Application No. 61/405,882, filed on Oct. 22, 2010 and entitled “POCHOXIME CONJUGATES USEFUL FOR THE TREATMENT OF HSP90 RELATED PATHOLOGIES”, the disclosures of which are hereby incorporated by reference in their entireties for all purposes. This application is also related to International Application No. PCT/US2007/017754, filed on Aug. 10, 2007 and entitled “Macrocyclic Compounds Useful as Inhibitors of Kinase and HSP90”; and International Application No. PCT/US2009/031149, filed on Jan. 15, 2009 and entitled “Synthesis of Resorcylic Acid Lactones Useful as Therapeutic Agents”, the disclosures of each of which are hereby incorporated by reference in their entireties for all purposes.

### FIELD OF THE INVENTION

The present invention is related to novel derivatives, analogs, and intermediates of the natural products radicicol, pochonins, pochoximes, and to their syntheses. The present invention is also related to the use of these compounds as inhibitors of kinases and of the enzyme family known as heat shock protein 90 (HSP90).

### BACKGROUND OF THE INVENTION

The heat shock protein 90 (HSP90) has emerged as an extremely promising therapeutic target in recent years.^{[1-3]} Despite the seemingly ubiquitous functions of this highly expressed chaperone, its role in stabilizing conformationally labile proteins has implications in diverse pathologies. Inhibitors of HSP90 have been shown to be broadly effective for a number of cancer indications,^{[4, 5]} neurodegenerative diseases,^{[6-10]} infectious diseases,^{[11]} and inflammation-related disorders.^{[12]} Two natural products, radicicol and geldanamycin (as shown in Scheme 1 below), both of which disrupt the ATPase activity of Hsp90, have been instrumental in understanding the role of HSP90 in oncogenic processes and the therapeutic potential of its inhibition.^{[13-15]} However, neither natural product has acceptable pharmacological properties for clinical application. Medicinal chemistry efforts have led to the discovery of novel scaffolds such as purines^{[16, 17]} (CNF-2024), resorcinol-isoxazoles^{[18-20]} (VER-52296) and 2-aminobenzamides^{[21-22]} (SNX 2112) which are currently in clinical or preclinical development.^{[5]} However, improving the pharmacological properties and potency of the natural pharmacophores remains important. Indeed, the most advanced clinical candidate is a semi synthetic derivative of geldanamycin, 17AAG (3, FIG. 1).^{[23, 24]} Another semi synthetic derivative with a dimethoxyhydroquinone functionality has recently been reported to have better pharmacological properties than 17AAG while acting as a prodrug.^{[25]}

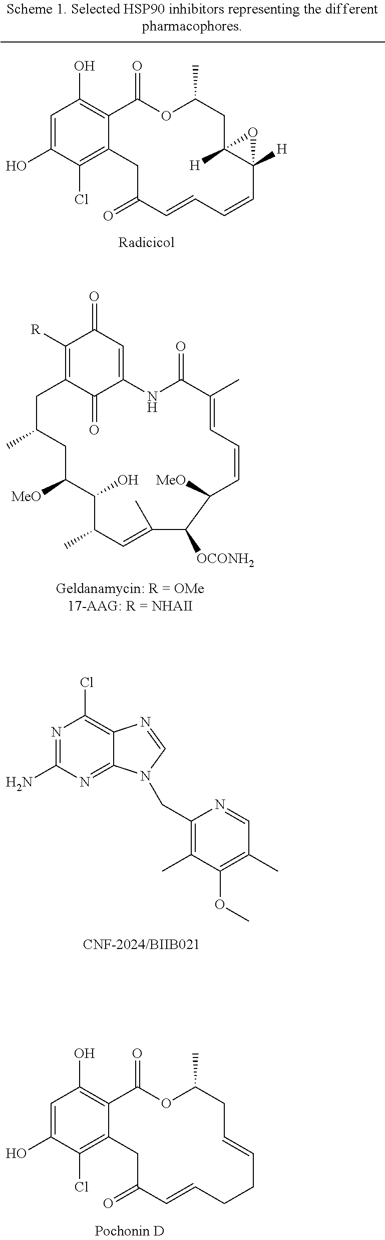

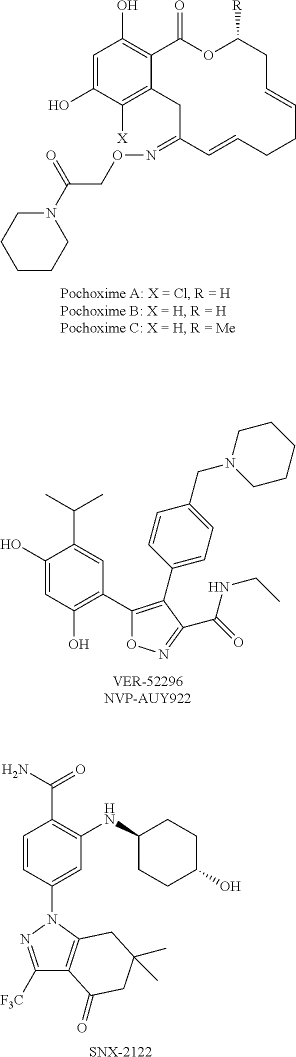

The present inventors have previously demonstrated^{[26]} that pochonin D represents a simplified pharmacophore of radicicol which recapitulates its activity. Furthermore, significant improvements in cellular efficacy could be achieved through the formation of oximes.^{[27]} In fact, pochoximes A, B, and C (as shown in Scheme 1 above), are amongst the most potent HSP90 inhibitors reported to date, inducing client protein degradation in SKBR3 cell lines at low nM concentration, and pochoxime A treatment leads to tumor regression in xenografts bearing BT474 breast tumor cells.

This application provides crystal structures obtained by co-crystallization of pochoxime A and B with human HSP90α and a compound library extending the diversity of the pochoximes as well as asymmetric synthesis of the pochoxime analogs with C-6 modifications.

### SUMMARY OF THE INVENTION

In one embodiment, the present invention provides a compound of formula (I), or a pharmaceutically acceptable salt, solvate, and/or prodrug thereof:

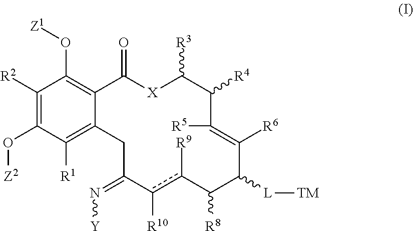

wherein:

X is O, S or NR;

Y is —OR, —O—(CH₂)ₘCOOR, —O—(CH₂)ₘCON(R)₂, —N(R)₂, —N(R)SOR or —N(R)SO₂R, wherein the groups bound to the nitrogen atom may be in Z- or E-configuration;

Z¹ and Z² are independently hydrogen or —(CH₂)—O—R^{Z};

R^{Z} is optionally substituted alkyl;

R¹ and R² are independently hydrogen, halogen, OR, N(R)₂, SR, azido, nitro, cyano, aliphatic, aryl, alkylaryl, arylalkyl, heterocyclyl, heteroaryl, —S(O)R, —S(O)₂R, —SO₂N(R)₂, —N(R)SO₂R, —N(CO)R, —N(CO)N(R)₂, —N(CO)OR, —O(CO)R, —(CO)R, —(CO)OR, —(CO)N(R)₂, —O(CO)OR, or —O(CO)N(R)₂;

R³, R⁴, R⁵, R⁶, R⁸, R⁹ and R¹⁰ are independently hydrogen, halogen, azido, nitro, cyano, aliphatic, alkylaryl, aralkyl, aryl, heteroalkyl, alkylheteroaryl, heterocyclyl, heteroaryl, OR, N(R)₂, SR, —O(CH₂)ₘN(R)C(O)(CH₂)ₚR, —O(CH₂)ₘOC(O)(CH₂)ₚR, —O(CH₂)ₘC(O)(CH₂)ₚN(R)₂, —O(CH₂)ₘC(O)(CH₂)ₚOR, —O(CH₂)ₘN(R)C(O)(CH₂)ₚOR, —O(CH₂)ₘN(R)C(O)(CH₂)ₚN(R)₂, —O(CH₂)ₘOC(O)(CH₂)ₚOR, —O(CH₂)ₘOC(O)(CH₂)ₚN(R)₂, —NR(CH₂)ₘN(R)C(O)(CH₂)ₚR, —NR(CH₂)ₘOC(O)(CH₂)ₚR, —NR(CH₂)ₘC(O)(CH₂)ₚN(R)₂, —NR(CH₂)ₘC(O)(CH₂)ₚOR, —NR(CH₂)ₘN(R)C(O)(CH₂)ₚOR, —NR(CH₂)ₘN(R)C(O)(CH₂)ₚN(R)₂, —NR(CH₂)ₘOC(O)(CH₂)ₚOR, —NR(CH₂)ₘOC(O)(CH₂)ₚN(R)₂, —(CH₂)ₘN(R)C(O)CH₂)ₚR, —(CH₂)ₘOC(O)(CH₂)ₚR, —(CH₂)ₘC(O)(CH₂)ₚN(R)₂, —(CH₂)ₘC(O)(CH₂)ₚOR, —(CH₂)ₘN(R)C(O)(CH₂)ₚOR, —(CH₂)ₘN(R)C(O)(CH₂)ₚN(R)₂, —(CH₂)ₘOC(O)(CH₂)ₚOR, —(CH₂)ₘOC(O)(CH₂)ₚN(R)₂, —(CH₂)ₘN₃, —O(CH₂)ₘN₃—(CH₂)ₘN(R)₂, —(CH₂)ₘOR, —(CH₂)ₘS(O)(CH₂)ₚR, —(CH₂)ₘS(O)₂(CH₂)ₚR, —(CH₂)ₘSO₂(CH₂)ₚN(R)₂, or —(CH₂)ₘN(R)SO₂(CH₂)ₚR; and

each R is independently hydrogen, aliphatic, amino, azido, cyano, nitro, alkylamino, dialkylamino, OH, alkoxy, carbonylamino, aminocarbonyl, alkoxycarbonyl, carbonyloxy, carboxy, acyl, aryl, alkaryl, arylalkyl including benzyl, heteroalkyl, heteroaryl, heterocyclyl, or a protecting group; or two R on the same nitrogen are taken together with the nitrogen to form a 5 to 8 membered heterocyclic or heteroaryl ring; wherein where a group contains more than one R substituent; wherein R is optionally substituted, and each R can be the same or different;

m and p are independently 0, 1, 2, 3, 4 or 5;

the dashed lines between the carbon atoms bearing R⁹ and R¹⁰ indicate either a single or a double bond, where the valence requirements are fulfilled by additional hydrogen atoms; and

L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and

TM is a targeting moiety that specifically binds with a biological situs under physiological conditions; or alternatively, L-TM is a group an oxygen- or nitrogen-based functional group.

In another embodiment, the present invention provides a pharmaceutical composition comprising a compound of the present invention and a pharmaceutically acceptable carrier.

In another embodiment, the present invention provides a method of treating a patient with a disease comprising administering to the patient with the disease an effective amount of a compound of the present invention, wherein the disease is mediated by kinases and Heat Shock Protein 90 (HSP90). In one embodiment, the disease is an autoimmune disease, inflammatory disease, neurological or neurodegenerative disease, cancer, cardiovascular disease, allergy, asthma, or a hormone-related disease.

### BRIEF DESCRIPTION OF THE DRAWINGS

FIG. 1. Co-crystal structure of radicicol (panel A—pdb: 1bgq) pochoxime A (panel B—pdb: 3inw) and pochoxime B (panel C—pdb: 3inx) with HSP90.

FIG. 2. Cellular efficacy of pochoximes A1B5C1D1 (IC₅₀). Depletion of Her-2 in SkBr3 cells were treated with the inhibitor for 18 h.

FIG. 3. Key differentiating proton NMR signals between pochonin E and its C-6 epimer (epi-pochonin E).

FIG. 4. X-ray crystallographic structure of pochoxime F 10a(6-R).

FIG. 5. Docking of pochoxime E 10b-R (top) and epi-pochonin E 10b-S (bottom) to HSP90.

### DETAILED DESCRIPTION OF THE INVENTION

The present invention provides pochoxime derivatives as herein described. In one embodiment, the present compounds have structural formulae (I), (II), (IIIa), (IIIb), and (IIIc) as well as any subgenus and species thereof, wherein a targeting moiety is appended to the allylic carbon 6 via a linkage moiety. Specifically, the allylic carbon 6 refers to the carbon atom (marked with *) covalently bonded to “L” as shown in structural formula (I) below:

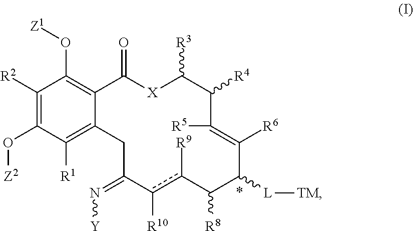

wherein the various substituents are the same as defined above in the Summary of the Invention section.

As used herein, the term “targeting moiety” can be a molecular moiety that specifically binds with a biological situs under physiological conditions. For example, the targeting moiety may bind to a defined population of cells or selected cell type. The targeting moiety may also bind a receptor, an oligonucleotide, an enzymatic substrate, an antigenic determinant, or other binding site present on or in the target cell or cell population. In some embodiments, the targeting moiety comprises an antibody, antibody fragment, or substance specific for a given receptor binding site. In other embodiments, the ligand, or targeting moiety comprises a receptor-specific peptide, carbohydrate, protein, lipid, nucleoside, peptide nucleic acid, or combinations thereof. In yet further embodiments the ligand or targeting moiety is an organic compound.

The targeting moiety can be used to enhance the pharmacological properties or to specifically capitalize on an active transport mechanism to enrich the concentration of the drug in specific cell types using conjugates such as glucose or biotin or peptides known to interact with cell surface receptors. The targeting group can be linked via an ether, ester, carbonate, thioether, thioester, amine, amide, urea, carbonate urea, thiourea, imine, hydrazine, hydrazone, and etc.

The term “nitrogen-based functional group”, as used herein, refers to an organic moiety containing a nitrogen and other atom(s) including any one or more of hydrogen, carbon, halogen, nitrogen, oxygen, sulfur, and etc., wherein the nitrogen atom is covalently attached to the allylic carbon 6. Examples of nitrogen-based functional group include, but are not limited to, amino, azide, N-alkyl substituted amino, N,N-dialkyl substituted amino, acyl substituted amino, and etc. wherein each of the alkyl and acyl is optionally substituted.

The term “nitrogen-based functional group”, as used herein, refers to an organic moiety containing an oxygen and other atom(s) including any one or more of hydrogen, carbon, halogen, nitrogen, oxygen, sulfur, and etc., wherein the oxygen atom is covalently attached to the allylic carbon 6. Examples of nitrogen-based functional group include, but are not limited to, hydroxyl, alkoxy, acyl substituted oxygen, and etc. wherein each of the alkyl and acyl is optionally substituted.

In one embodiment, the present invention provides a compound of formula (I) as described above, or a pharmaceutically acceptable salt, solvate, and/or prodrug thereof.

In one embodiment, the structural Formula (I) does not include the compound listed in Table X below:

TABLE X

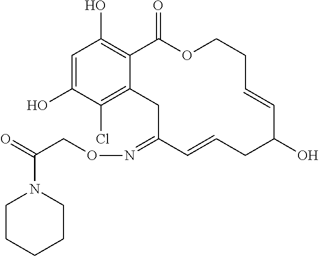 [SMILES: O=C1OCC/C=C/C(O)C/C=C/C(=N/OCC(=O)N2CCCCC2)Cc2c(Cl)c(O)cc(O)c21]

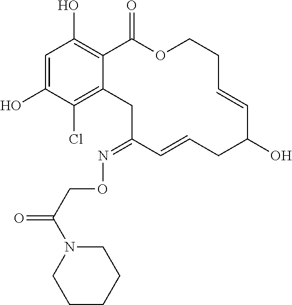 [SMILES: O=C1OCC/C=C/C(O)C/C=C/C(=N\OCC(=O)N2CCCCC2)Cc2c(Cl)c(O)cc(O)c21]

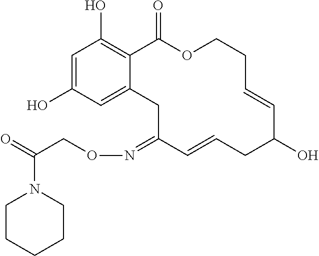 [SMILES: O=C1OCC/C=C/C(O)C/C=C/C(=N/OCC(=O)N2CCCCC2)Cc2cc(O)cc(O)c21]

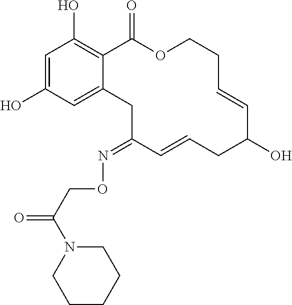 [SMILES: O=C1OCC/C=C/C(O)C/C=C/C(=N\OCC(=O)N2CCCCC2)Cc2cc(O)cc(O)c21]

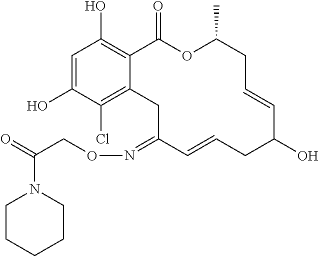 [SMILES: C[C@@H]1C/C=C/C(O)C/C=C/C(=N/OCC(=O)N2CCCCC2)Cc2c(Cl)c(O)cc(O)c2C(=O)O1]

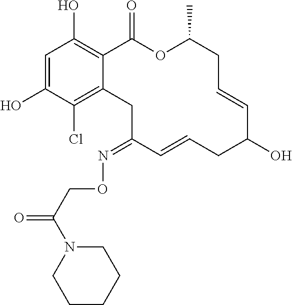 [SMILES: C[C@@H]1C/C=C/C(O)C/C=C/C(=N\OCC(=O)N2CCCCC2)Cc2c(Cl)c(O)cc(O)c2C(=O)O1]

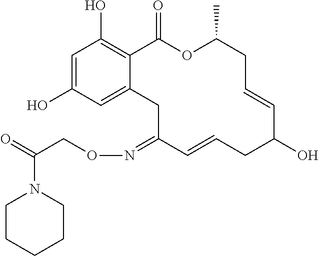 [SMILES: C[C@@H]1C/C=C/C(O)C/C=C/C(=N/OCC(=O)N2CCCCC2)Cc2cc(O)cc(O)c2C(=O)O1]

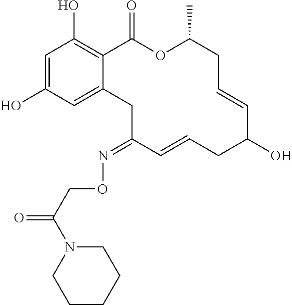 [SMILES: C[C@@H]1C/C=C/C(O)C/C=C/C(=N\OCC(=O)N2CCCCC2)Cc2cc(O)cc(O)c2C(=O)O1]

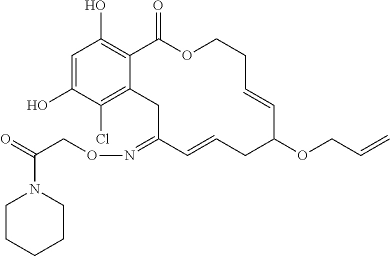 [SMILES: C=CCOC1/C=C/CCOC(=O)c2c(O)cc(O)c(Cl)c2CC(=N\OCC(=O)N2CCCCC2)/C=C/C1]

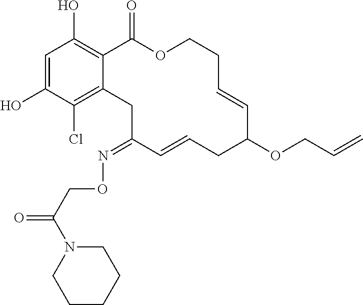 [SMILES: C=CCOC1/C=C/CCOC(=O)c2c(O)cc(O)c(Cl)c2CC(=N/OCC(=O)N2CCCCC2)/C=C/C1]

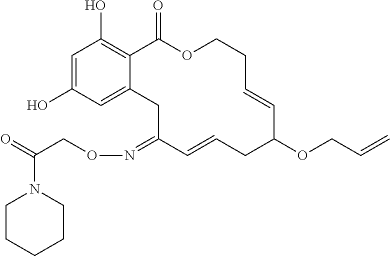 [SMILES: C=CCOC1/C=C/CCOC(=O)c2c(O)cc(O)cc2CC(=N\OCC(=O)N2CCCCC2)/C=C/C1]

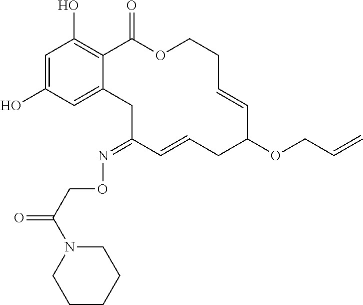 [SMILES: C=CCOC1/C=C/CCOC(=O)c2c(O)cc(O)cc2CC(=N/OCC(=O)N2CCCCC2)/C=C/C1]

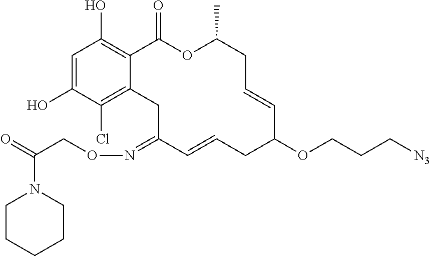 [SMILES: C[C@@H]1C/C=C/C(OCCCN=[N+]=[N-])C/C=C/C(=N/OCC(=O)N2CCCCC2)Cc2c(Cl)c(O)cc(O)c2C(=O)O1]

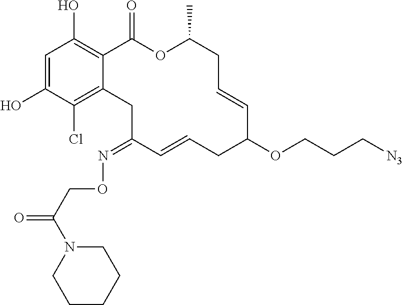 [SMILES: C[C@@H]1C/C=C/C(OCCCN=[N+]=[N-])C/C=C/C(=N\OCC(=O)N2CCCCC2)Cc2c(Cl)c(O)cc(O)c2C(=O)O1]

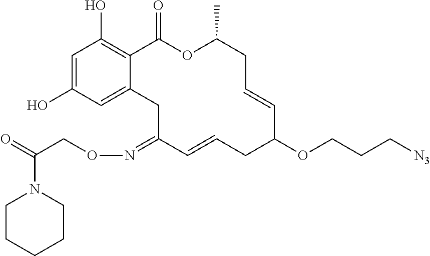 [SMILES: C[C@@H]1C/C=C/C(OCCCN=[N+]=[N-])C/C=C/C(=N/OCC(=O)N2CCCCC2)Cc2cc(O)cc(O)c2C(=O)O1]

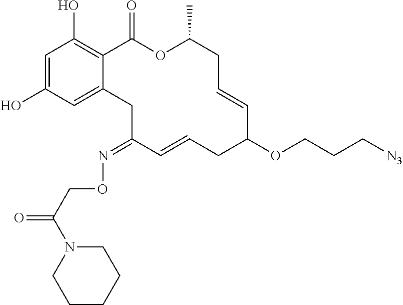 [SMILES: C[C@@H]1C/C=C/C(OCCCN=[N+]=[N-])C/C=C/C(=N\OCC(=O)N2CCCCC2)Cc2cc(O)cc(O)c2C(=O)O1]

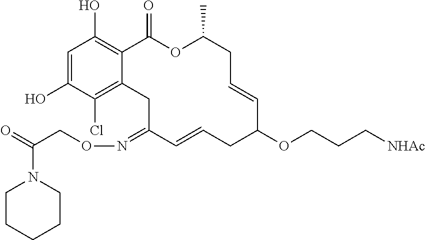 [SMILES: CC(=O)NCCCOC1/C=C/C[C@@H](C)OC(=O)c2c(O)cc(O)c(Cl)c2CC(=N\OCC(=O)N2CCCCC2)/C=C/C1]

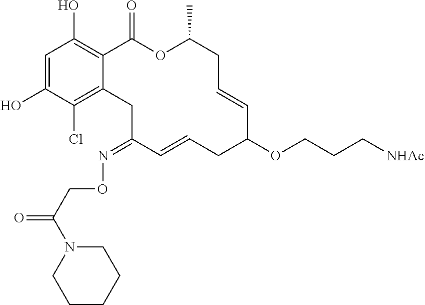 [SMILES: CC(=O)NCCCOC1/C=C/C[C@@H](C)OC(=O)c2c(O)cc(O)c(Cl)c2CC(=N/OCC(=O)N2CCCCC2)/C=C/C1]

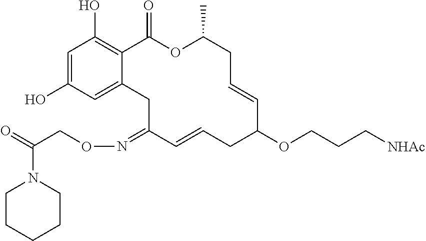 [SMILES: CC(=O)NCCCOC1/C=C/C[C@@H](C)OC(=O)c2c(O)cc(O)cc2CC(=N\OCC(=O)N2CCCCC2)/C=C/C1]

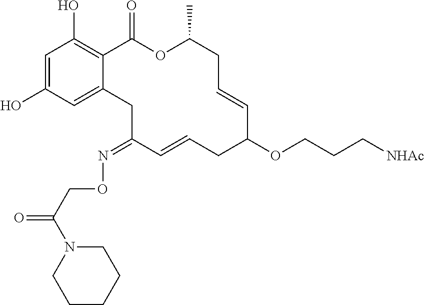 [SMILES: CC(=O)NCCCOC1/C=C/C[C@@H](C)OC(=O)c2c(O)cc(O)cc2CC(=N/OCC(=O)N2CCCCC2)/C=C/C1]

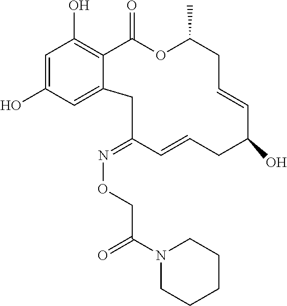 [SMILES: C[C@@H]1C/C=C/[C@@H](O)C/C=C/C(=N\OCC(=O)N2CCCCC2)Cc2cc(O)cc(O)c2C(=O)O1]

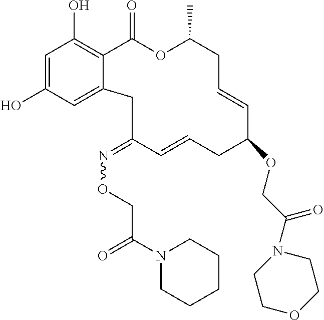 [SMILES: C[C@@H]1C/C=C/[C@@H](OCC(=O)N2CCOCC2)C/C=C/C(=NOCC(=O)N2CCCCC2)Cc2cc(O)cc(O)c2C(=O)O1]

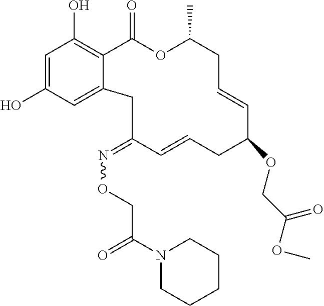 [SMILES: COC(=O)CO[C@@H]1/C=C/C[C@@H](C)OC(=O)c2c(O)cc(O)cc2CC(=NOCC(=O)N2CCCCC2)/C=C/C1]

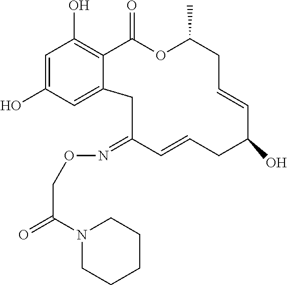 [SMILES: C[C@@H]1C/C=C/[C@@H](O)C/C=C/C(=N/OCC(=O)N2CCCCC2)Cc2cc(O)cc(O)c2C(=O)O1]

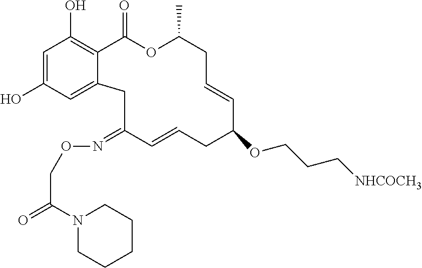

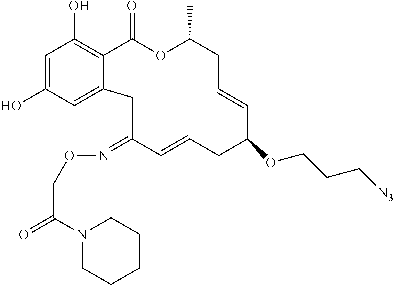 [SMILES: C[C@@H]1C/C=C/[C@@H](OCCCN=[N+]=[N-])C/C=C/C(=N/OCC(=O)N2CCCCC2)Cc2cc(O)cc(O)c2C(=O)O1]

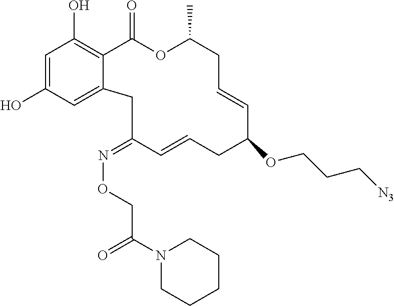 [SMILES: C[C@@H]1C/C=C/[C@@H](OCCCN=[N+]=[N-])C/C=C/C(=N\OCC(=O)N2CCCCC2)Cc2cc(O)cc(O)c2C(=O)O1]

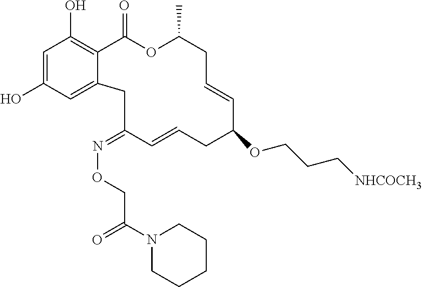

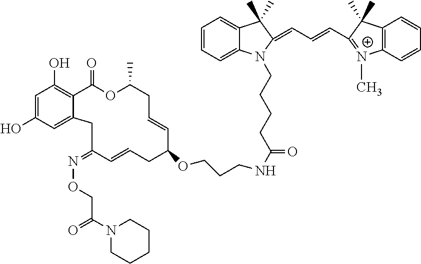

In one embodiment of structural Formula (I), X is O or NR.

In one embodiment of structural Formula (I), Y is —OR, —O—(CH₂)ₘCOOR or —O—(CH₂)ₘCON(R)₂.

In one embodiment of structural Formula (I), R¹ and R² are independently hydrogen, halogen, or lower alkyl.

In one embodiment of structural Formula (I), R¹ is hydrogen, halogen, or lower alkyl; and R² is hydrogen.

In one embodiment of structural Formula (I), R^{Z} is lower alkyl, alkoxy-substituted lower alkyl, or aryl-substituted lower alkyl. In one embodiment, R^{Z} is methyl, ethyl, isopropyl, n-propyl, n-butyl, isobutyl, t-butyl, methoxy-ethyl, methoxy-methyl, chloromethyl, or benzyl.

In one embodiment of structural Formula (I), L-TM is an oxygen or nitrogen-based functional group.

In one embodiment of structural Formula (I), the compound can be represented by structural formula (II):

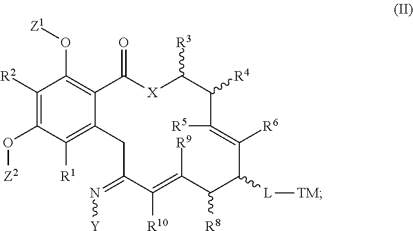

wherein,

X is O, S or NR;

Y is —OR, —O—(CH₂)ₘCOOR, —O—(CH₂)ₘCON(R)₂, —N(R)₂, —N(R)SOR or —N(R)SO₂R, wherein the groups bound to the nitrogen atom may be in Z- or E-configuration;

Z¹ and Z² are independently hydrogen or —(CH₂)—O—R^{Z};

R^{Z} is optionally substituted alkyl;

R¹ and R² are independently hydrogen, halogen, or alkyl;

R³, R⁴, R⁵, R⁶, R⁸, R⁹ and R¹⁰ are independently hydrogen, halogen, or alkyl;

L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and

TM is a targeting moiety that specifically binds with a biological situs under physiological conditions; or alternatively, L-TM is a group an oxygen- or nitrogen-based functional group;

In one embodiment of structural Formula (II), the compound can be represented by structural Formula (IIIa):

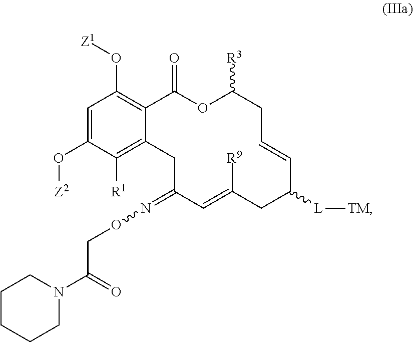

wherein, Z¹ and Z² are —(CH₂)—O—R^{Z}; R^{Z} is optionally substituted alkyl; R¹ is H, halogen, or lower alkyl; R³ and R⁹ are independently H or lower alkyl; and L-TM is an oxygen-based functional group.

In one embodiment of structural Formula (II), the compound can be represented by structural Formula (IIIb):

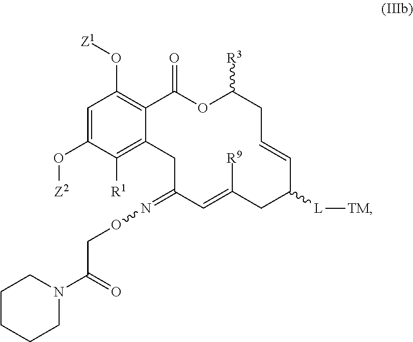

wherein, Z¹ and Z² are —(CH₂)—O—R^{Z}; R^{Z} is hydrogen or optionally substituted alkyl; R¹ is H, halogen, or lower alkyl; R³ and R⁹ are independently H or lower alkyl; and L-TM is a nitrogen-based functional group.

In one embodiment of structural Formula (II), the compound can be represented by structural Formula (IIIc):

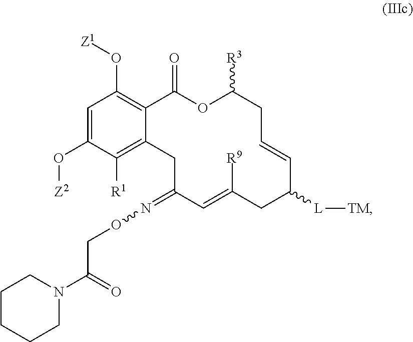

wherein, Z¹ and Z² are —(CH₂)—O—R^{Z}; R^{Z} is hydrogen or optionally substituted alkyl; R¹ is H, halogen, or lower alkyl; R³ and R⁹ are independently H or lower alkyl; and L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and TM is a targeting moiety that specifically binds with a biological situs under physiological conditions.

In certain specific embodiments, the present invention provides a compound selected from the group consisting of

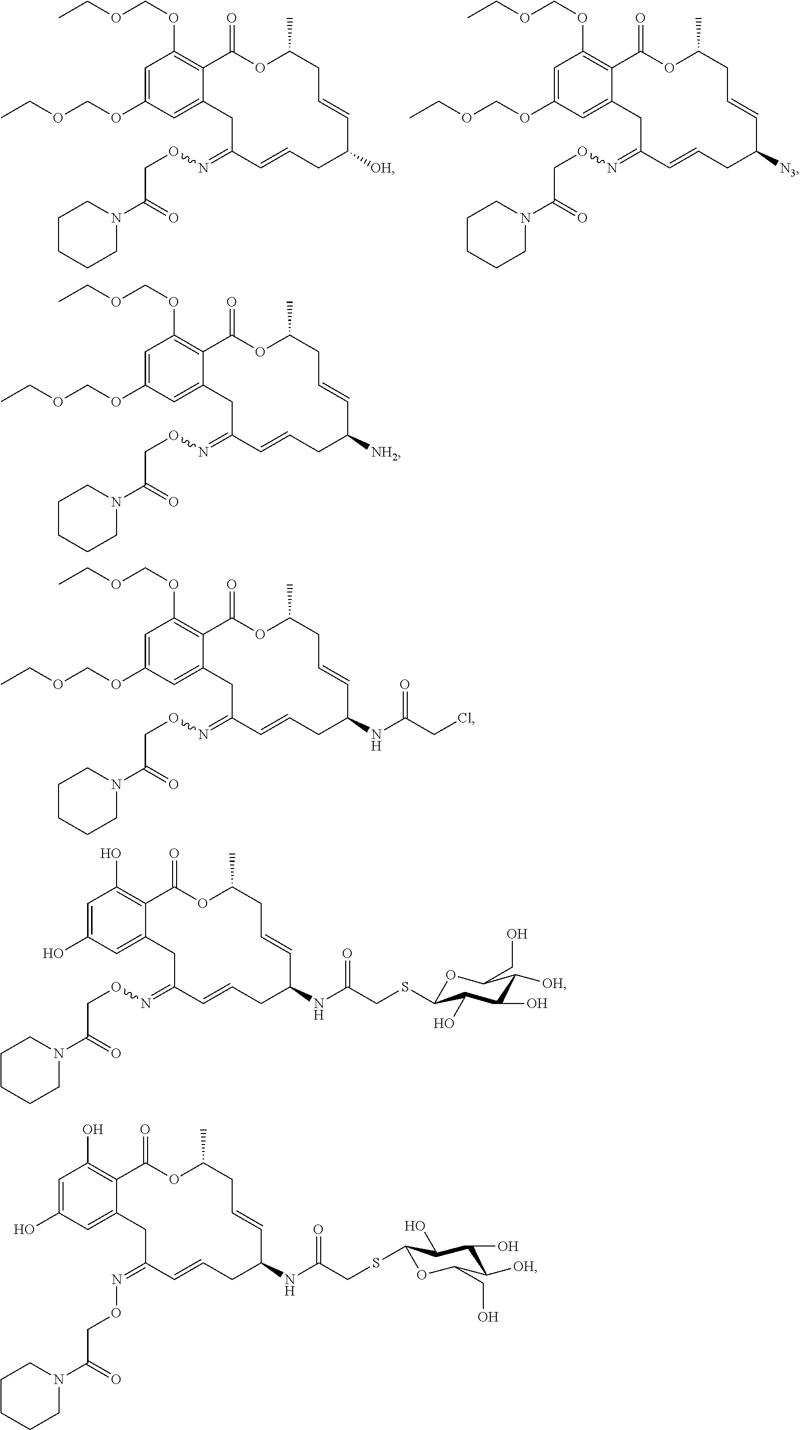

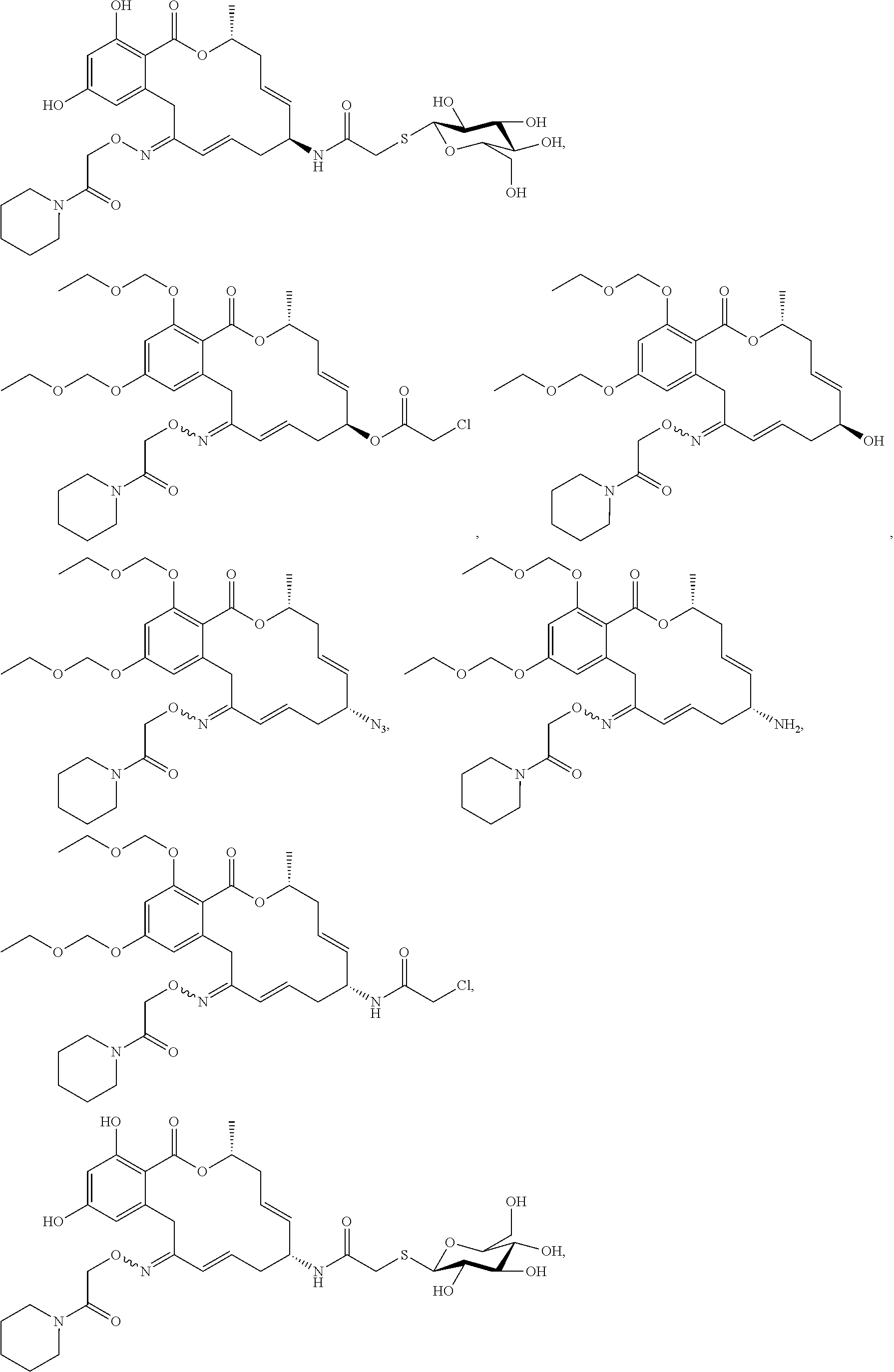

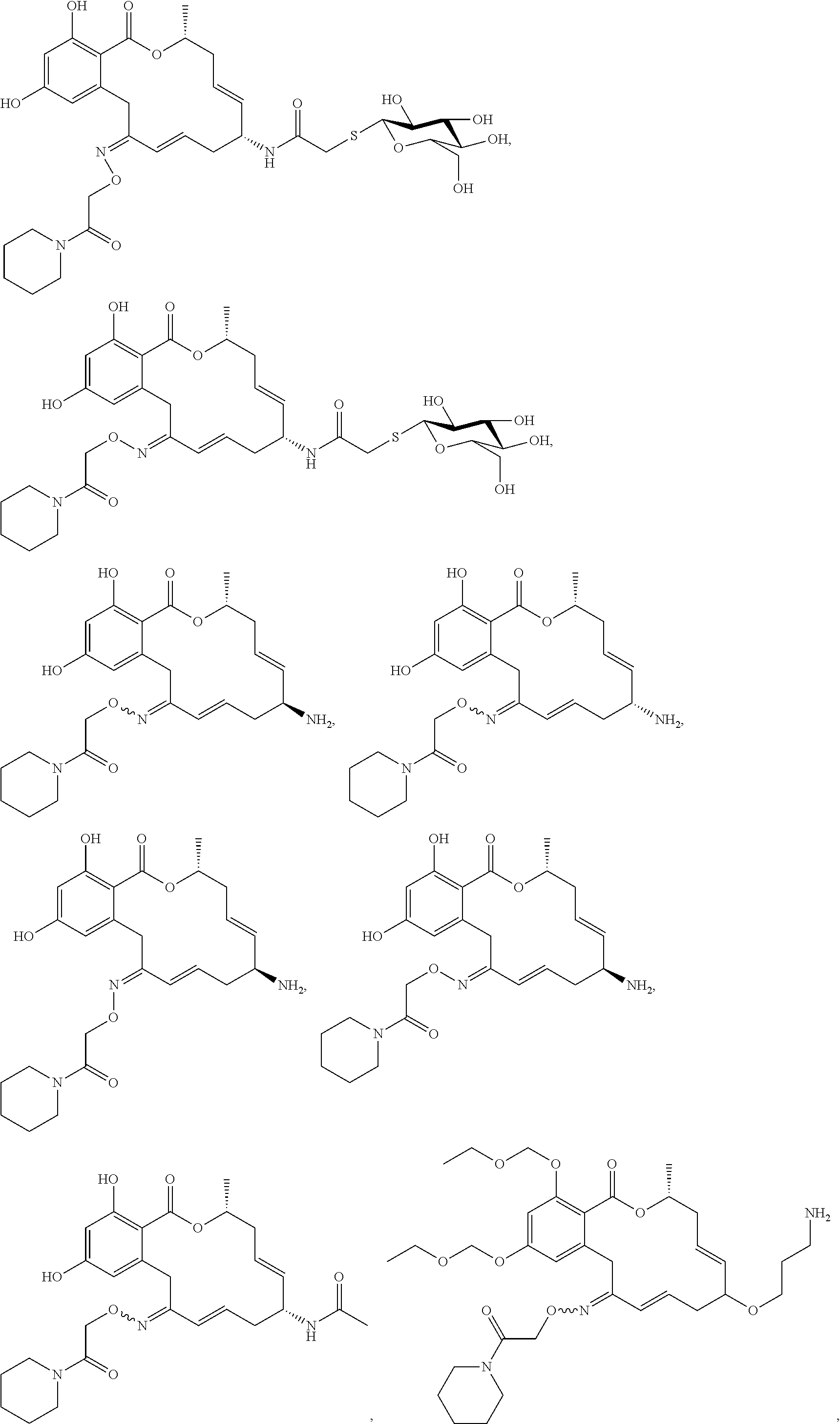

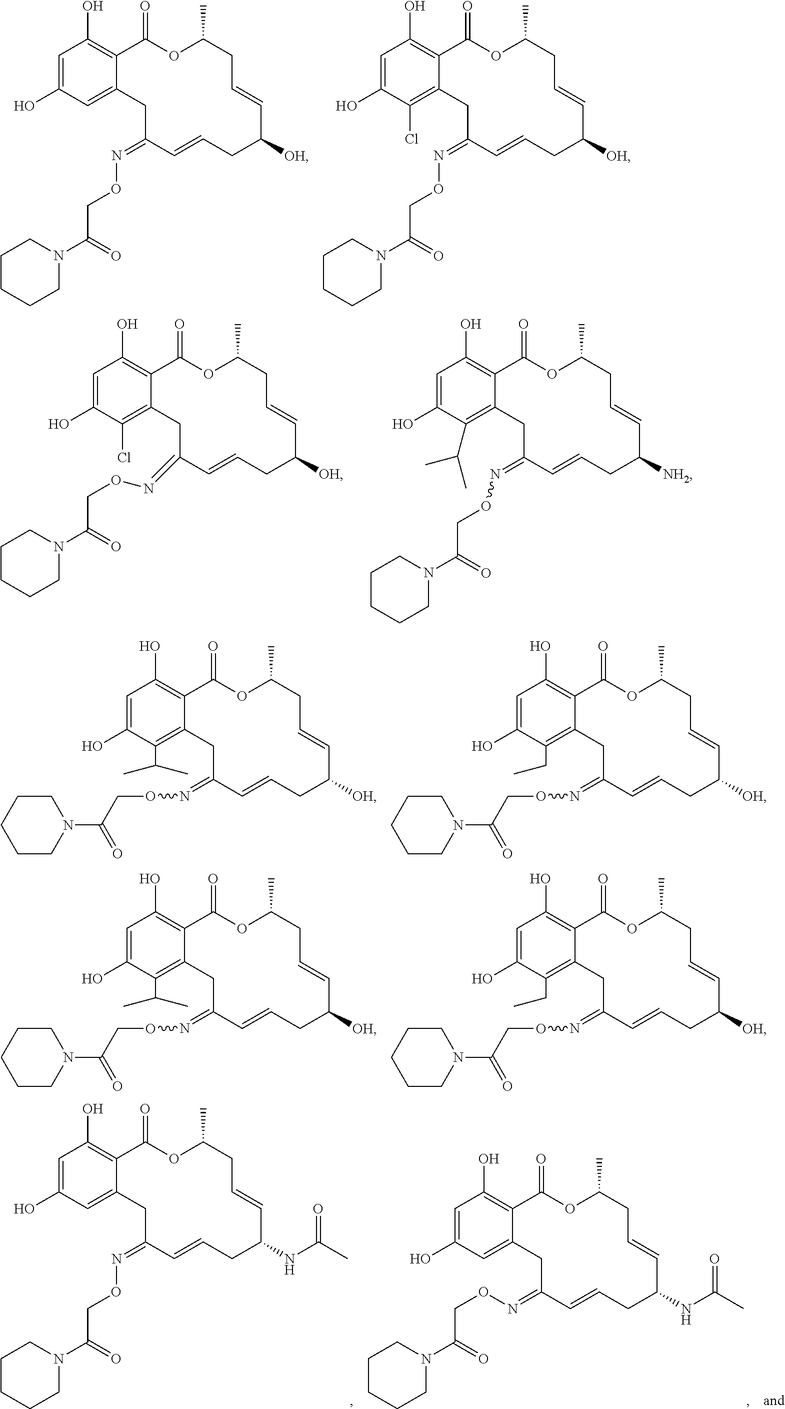

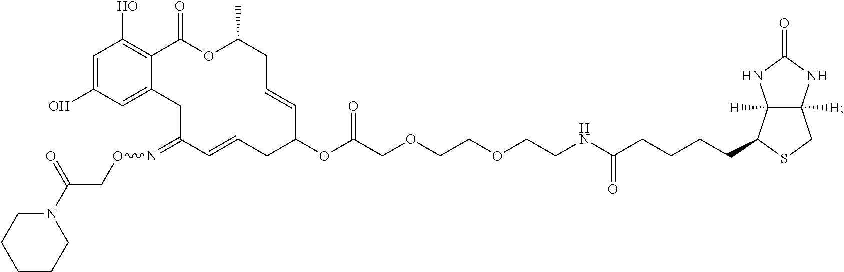

or a pharmaceutically acceptable salt, solvate, and/or prodrug thereof.

The terms “a” and “an” do not denote a limitation of quantity, but rather denote the presence of at least one of the referenced item. The terms “a” and “an” are used interchangeable with “one or more” or “at least one”. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The terms “compound(s) of the present invention”, “these compounds”, “such compound(s)”, “the compound(s)”, and “the present compound(s)” refer to compounds encompassed by structural formulae disclosed herein, e.g., Formula (I), (II), (IIIa), (IIIb), and (IIIc), including any specific compounds within these formulae whose structure is disclosed herein. Compounds may be identified either by their chemical structure and/or chemical name.

When the chemical structure and chemical name conflict, the chemical structure is determinative of the identity of the compound. Furthermore, the present compounds can inhibit the biological activity of a CK2 protein, and thereby is also referred to herein as an “inhibitor(s)” or “CK2 inhibitor(s)”. Compounds of Formula (I), (II), (IIIa), (IIIb), and (IIIc), including any specific compounds described herein are exemplary “inhibitors”. The descriptions of compounds of the invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Whenever a term in the specification is identified as a range (i.e. C₁₋₆ alkyl), the range independently refers to each element of the range. As a non-limiting example, C₁₋₆ alkyl means, independently, C₁, C₂, C₃, C₄, C₅, or C₆ alkyl. Similarly, when one or more substituents are referred to as being “independently selected from” a group, this means that each substituent can be any element of that group, and any combination of these groups can be separated from the group. For example, if R¹ and R² can be independently selected from X, Y and Z, this separately includes the groups R¹ is X and R² is X; R¹ is X and R² is Y; R¹ is X and R² is Z; R¹ is Y and R² is X; R¹ is Y and R² is Y; R¹ is Y and R² is Z; R¹ is Z and R² is X; R¹ is Z and R² is Y; and R¹ is Z and R² is Z.

The term “aliphatic” as used herein means straight-chain, branched or cyclic typically of C₁ to C₁₈, and in certain embodiment of C₁ to C₁₀ or of C₁ to C₆, hydrocarbons which are completely saturated or which contain one or more units of unsaturation but which are not aromatic. For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl. The terms “alkyl”, “alkoxy”, “hydroxyalkyl”, “alkoxyalkyl”, and “alkoxycarbonyl”, used alone or as part of a larger moiety includes both straight and branched chains containing one to twelve carbon atoms. The terms “alkenyl” and “alkynyl” used alone or as part of a larger moiety shall include both straight and branched chains containing two to twelve carbon atoms. The term “cycloalkyl” used alone or as part of a larger moiety shall include cyclic C₃-C₁₂ hydrocarbons which are completely saturated or which contain one or more units of unsaturation, but which are not aromatic, including but not limited to cyclopropyl, cyclobutyl, cyclopentyl, cyclohexyl, cycloheptyl and cyclooctyl. Aliphatic groups can be optionally substituted with one or more moieties, including but not limited to, alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamoyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “alkyl” as used herein, unless otherwise specified, refers to a saturated straight, branched, or cyclic, primary, secondary, or tertiary hydrocarbon, including but not limited to groups typically of C₁ to C₁₈ and in certain embodiment of C₁ to C₁₀ or of C₁ to C₆, and specifically includes methyl, ethyl, propyl, isopropyl, butyl, isobutyl, t-butyl, pentyl, cyclopentyl, isopentyl, neopentyl, hexylisohexyl, cyclohexyl, cyclohexylmethyl, 3-methylpentyl, 2, 2-dimethylbutyl and 2,3-dimethylbutyl. Alkyl groups may be substituted as noted above for the term “aliphatic.”

The term “lower alkyl,” as used herein, and unless otherwise specified, refers to optionally substituted C₁ to C₆ saturated straight, branched, or if appropriate, a cyclic (for example, cyclopropyl) alkyl group, including both substituted and unsubstituted forms.

Illustrative examples of alkyl groups are methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene et al., Protective Groups in Organic Synthesis, John Wiley & Sons, 3^{rd} Ed., 1999.

The term “halo” or “halogen”, as used herein, includes chloro, bromo, iodo, and fluoro.

The term “chiral” as used herein includes a compound that has the property that it is not superimposable on its mirror image.

The term “tautomer” as used herein refers to alternate structures which are recognized in the art to be in equilibrium with the depicted structure. For example, the enol structure below is a tautomer of the ketone structure and recognized to be in equilibrium with the ketone structure.

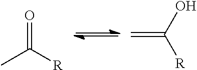

As used herein, the term “solvate” or “pharmaceutically acceptable solvate,” is a solvate formed from the association of one or more solvent molecules to one or more molecules of a compound of any one of formulas I, I′, II, II′, III, III′, IV or V or the compounds depicted in Table 1. The term solvate includes hydrates (e.g., hemi-hydrate, mono-hydrate, dihydrate, trihydrate, tetrahydrate, and the like).

The term “alkylthio” refers to a straight or branched chain alkylsulfide of the number of carbons specified, such as for example, C₁₋₄alkylthio, ethylthio, —S-alkyl, —S-alkenyl, —S-alkynyl, etc.

The terms “alkylamino” or “arylamino” refer to an amino group that has one or two alkyl or aryl substituents, respectively. Unless otherwise specifically stated in this application, when alkyl is a suitable moiety, then it is a lower alkyl, whether substituted or unsubstituted.

The term “alkylsulfonyl” means a straight or branched alkylsulfone of the number of carbon atoms specified, as for example, C₁₋₆ alkylsulfonyl or methylsulfonyl.

The term “alkoxycarbonyl” refers to a straight or branched chain ester of a carboxylic acid derivative of the number of carbon atoms specified, such as for example, a methoxycarbonyl, MeOCO—.

As used herein, the term “nitro” means —NO₂; the term “sulfhydryl” means —SH; and the term “sulfonyl” means —SO₂.

The terms “alkenyl” and “alkynyl” refer to alkyl moieties, including both substituted and unsubstituted forms wherein at least one saturated C—C bond is replaced by a double or triple bond. Thus, C₂₋₆ alkenyl may be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Similarly, C₂₋₆ alkynyl may be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkylene” includes a saturated, straight chain, divalent alkyl radical of the formula —(CH₂)ₙ—, wherein “n” may be any whole integer from 1 to 12.

“Alkyl”, “alkoxy”, “alkenyl”, “alkynyl”, etc., includes both straight chain and branched groups. However, reference to an individual radical such as “propyl” embraces only that straight-chain radical, whereas a branched chain isomer such as “isopropyl” is specifically termed such.

The term “aryl” as used herein and unless otherwise specified refers to any stable monocyclic, bicyclic, or tricyclic carbon ring of up to 8 members in each ring, wherein at least one ring is aromatic as defined by the Huckel 4n+2 rule, and especially phenyl, biphenyl, or naphthyl. The term includes both substituted and unsubstituted moieties. The aryl group can be optionally substituted with one or more moieties. Examples of substituents include alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfate, sulfonyl, sulfanyl, sulfinyl, sulfamoyl, ester, carboxylic acid, amide, phosphate, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., “Protective Groups in Organic Synthesis,” John Wiley and Sons, Second Edition, 1991.

The term “alkaryl” or “alkylaryl” refers to an alkyl group with an aryl substituent or an alkyl group linked to the molecule through an aryl group as defined herein. The term “aralkyl” or “arylalkyl” refers to an aryl group substituted with an alkyl substituent or linked to the molecule through an alkyl group as defined above.

The term “alkoxy” means a straight or branched chain alkyl group having an attached oxygen radical, the alkyl group having the number of carbons specified or any number within this range. For example, a “—O-alkyl”, C₁₋₄ alkoxy, methoxy, etc.

The term “acyl” includes a group of the formula C(O)R′, wherein R′ is an straight, branched, or cyclic alkyl (including lower alkyl), carboxylate residue of an amino acid, aryl including phenyl, heteroaryl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl. Aryl groups optimally comprise a phenyl group. In non-limiting embodiments, acyl groups include acetyl, trifluoroacetyl, methylacetyl, cyclopropylacetyl, cyclopropyl-carboxy, propionyl, butyryl, isobutyryl, hexanoyl, heptanoyloctanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chloro sulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, 4-phenylbenzoyl.

The term “acylamino” includes a group having a structure of “—N(R′)—C(═O)—R′”, wherein each R′ is independently as defined above.

The term “ester” includes a group of the structure “—C(═O)—O—R′” or “—O—C(═O)—R′”, wherein R′ is an straight, branched, or cyclic alkyl (including lower alkyl), carboxylate residue of an amino acid, aryl including phenyl, heteroaryl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl. Aryl groups optimally comprise a phenyl group.

The term “heteroatom” includes an atom other than carbon or hydrogen in the structure of a heterocyclic compound, nonlimiting examples of which are nitrogen, oxygen, sulfur, phosphorus or boron.

The term “carbonyl” or “includes a group of the structure “—C(═O)—X—R′” or “X—C(═O)—R′”, where X is O, S, or a bond, and each R is independently as defined above for “ester”.

The term “heterocycle”, “heterocyclyl”, or “heterocyclic” as used herein includes non-aromatic ring systems having four to fourteen members, preferably five to ten, in which one or more ring carbons, preferably one to four, are each replaced by a heteroatom. Examples of heterocyclic rings include 3-1H-benzimidazol-2-one, (1-substituted)-2-oxo-benzimidazol-3-yl, 2-tetrahydro-furanyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetra-hydro-thiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquinolinyl, and pyrido [3,4-d]pyrimidinyl. The term “heteroaryl” also refers to rings that are optionally substituted. The term “heteroaryl” may be used interchangeably with the term “heteroaryl ring” or the term “heteroaromatic”.

The term “amino” as used herein unless otherwise specified, includes a moiety represented by the structure “—N(R)₂”, and includes primary, secondary and tertiary amines optionally substituted by alkyl, aryl, heterocyclyl, and/or sulfonyl groups. Thus (R)₂ may represent two hydrogen atoms, two alkyl moieties, or one hydrogen and one alkyl moiety.

The term “amido” as used herein includes an amino-substituted carbonyl, while the term “amidino” means a group having the structure “—C(═NH)—NH₂”.

The term “counterion” refers to a negatively or positively charged ionic species that accompanies an oppositely charged ionic species in order to maintain electric neutrality. Negatively charged counterions include inorganic counterions and organic counterions, including but not limited to, chloro, bromo, iodo, fluoro, phosphate, acetate, formate, sulfonate, trifluoroacetate acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Positively charged counterions include, but are not limited to, alkali metal (e.g., sodium and potassium), alkaline earth metal (e.g., magnesium), ammonium and N⁺(C₁₋₄ alkyl)₄ counterions.

The term “quaternary amine” as used herein includes quaternary ammonium salts that have a positively charged nitrogen. They are formed by the reaction between a basic nitrogen in the compound of interest and an appropriate quaternizing agent such as, for example, methyliodide or benzyliodide. Appropriate counterions accompanying a quaternary amine include acetate, trifluoroacetate, chloro, bromo and iodo ions.

The term “substituted” includes multiple degrees of substitution by one or more named substituents such as, for example, halo, hydroxyl, thio, alkyl, alkenyl, alkynyl, nitro, cyano, azido, amino, carboxamido, etc. Where multiple substituent possibilities exist, the compound can be substituted by one or more of the disclosed or claimed substituent groups, independently from one another, and taken singly or in plural.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “protecting group” as used herein refers to a group that may be attached to a reactive group, including heteroatoms such as oxygen or nitrogen, to prevent the reactive group from participating in a reaction. Any protecting groups taught in for example, in Greene et al., Protective Groups in Organic Synthesis, John Wiley & Sons, 3^{rd} Ed., 1999, may be used. Examples of suitable protecting groups include but are not limited to alkoxyalkyl groups such as ethoxymethyl and methoxymethyl; silyl protecting groups, such tert-butyldimethyl silyl (TBS), phenyldimethylsilyl, trimethylsilyl (TMS), 2-trimethylsilylethoxymethyl (SEM) and 2-trimethylsilylethyl; and benzyl and substituted benzyl.

It should be understood that the various possible stereoisomers of the groups mentioned above and herein are within the meaning of the individual terms and examples, unless otherwise specified. As an illustrative example, “1-methyl-butyl” exists in both (R) and the (S) form, thus, both (R)-1-methyl-butyl and (S)-1-methyl-butyl is covered by the term “1-methyl-butyl”, unless otherwise specified.

The term “patient” includes human and veterinary subjects.

An “effective amount” is the quantity of compound in which a beneficial outcome is achieved when the compound is administered to a patient or alternatively, the quantity of compound that possesses a desired activity in vivo or in vitro. In the case of proliferative disorders, a beneficial clinical outcome includes reduction in the extent or severity of the symptoms associated with the disease or disorder and/or an increase in the longevity and/or quality of life of the patient compared with the absence of the treatment. For example, for a subject with cancer, a “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the patient, such as general health, age, sex, body weight and tolerance to drugs. It will also depend on the degree, severity and type of proliferative disorder. The skilled artisan will be able to determine appropriate dosages depending on these and other factors.

The term “kinase-inhibiting amount” as used herein, refers to an amount of the compound that inhibits a kinase enzyme compared to a control as tested by the methods described herein.

The term “HSP 90-inhibiting amount” as used herein, refers to an amount of the compound that inhibits HSP90 compared to a control as tested by the methods described herein.

The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

The term “cancer” includes, but is not limited to, solid tumors and blood borne tumors and include, but is not limited to, the following cancers: breast, ovary, cervix, prostate, testis, genitourinary tract, esophagus, larynx, glioblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia. The term “cancer” includes primary cancer, cancers secondary to treatment, and metastatic cancers.

The term “pharmaceutically acceptable carrier” refers to a non-toxic carrier, adjuvant, or vehicle that may be administered to a patient, together with a compound of this invention, and which does not destroy the pharmacological activity thereof.

“Excipient” refers to a diluent, adjuvant, vehicle, or carrier with which a compound is administered.

The term “HSP90-mediated disease” or “HSP90-mediated condition” refers to a condition in which HSP90 is known to pay a role. The conditions include but are not limited to inflammatory disorders, abnormal cellular proliferation, autoimmune disorders, ischemia, fibrogenetic disorders including but not limited to scleroderma, polymyositis, systemic lupus, rheumatoid arthritis, liver cirrhosis, keloid formation, interstitial nephritis, and pulmonary fibrosis. (Strehlow, WO 02/02123; PCT/US01/20578).

The terms “pharmaceutically acceptable salt” and “prodrug” are used throughout the specification to describe any pharmaceutically acceptable form (such as a salt, an ester, a phosphate ester, salt of an ester or a related group) of a compound which, upon administration to a patient, provides the compound described in the specification. In cases where compounds are sufficiently basic or acidic to form stable nontoxic acid or base salts, administration of the compounds as salts may be appropriate. The term pharmaceutically acceptable salts or complexes refers to salts or complexes that retain the desired biological activity of the compounds of the present invention and exhibit minimal undesired toxicological effects.

Non-limiting examples of such salts are (a) acid addition salts formed with inorganic acids such as sulfate, nitrate, hydrochloric, phosphate, and the like. For example, salts formed by the addition of hydrochloric acid, hydrobromic acid, sulfuric acid, phosphoric acid, nitric acid, and the like. In addition, salts formed with organic acids are encompassed by the invention, including tosylate, methanesulfonate, acetate, citrate, malonate, tartrate, succinate, benzoate, ascorbate, α-ketoglutarate, and α-glycerophosphate salts, such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The invention also encompasses (b) base addition salts, including formed with metal cations such as zinc, calcium, bismuth, barium, magnesium, aluminum, copper, cobalt, nickel, cadmium, sodium, potassium, lithium and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate.

Pharmaceutically acceptable salts may be obtained using standard procedures well known in the art, for example by reacting a sufficiently basic compound such as an amine with a suitable acid affording a physiologically acceptable anion.

Pharmaceutically acceptable “prodrugs” refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. For example, a suitable prodrug may be an ester or an amide of a carboxylic acid that is hydrolyzed to form the acid. Non-limiting examples of prodrugs include but are not limited to alkyl or aralkyl esters or amides, including methyl, ethyl, propyl, benzyl and substituted benzyl esters or amides. Prodrugs also comprise phosphate esters of the compounds.

### Stereoisomerism and Polymorphism

Compounds of the present invention having a chiral center may exist in and be isolated in optically active and racemic forms. The present invention encompasses any racemic, optically-active, diastereomeric, polymorphic, or stereoisomeric form, or mixtures thereof, of a compound of the invention, which possess the useful properties described herein.

In one embodiment, the compounds are prepared in optically active form by asymmetric synthesis using the processes described herein or synthetic transformations known to those skilled in the art.

Other methods to obtain optically active materials are known in the art, and include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enatiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent; or

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In another aspect, the present invention provides pharmaceutical compositions (i.e., formulations). The pharmaceutical compositions can comprise a compound of the present invention, as described herein, which is admixed with at least one pharmaceutically acceptable excipient or carrier. Frequently, the composition comprises at least two pharmaceutically acceptable excipients or carriers.

While the compositions and methods of the present invention will typically be used in therapy for human patients, they may also be used in veterinary medicine to treat similar or identical diseases. The compositions may, for example, be used to treat mammals, including, but not limited to, primates and domesticated mammals. The compositions may, for example be used to treat herbivores. The compositions of the present invention include geometric and optical isomers of one or more of the drugs, wherein each drug is a racemic mixture of isomers or one or more purified isomers.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

Any suitable formulation of a compound described above can be prepared for administration. Any suitable route of administration may be used, including, but not limited to, oral, parenteral, intravenous, intramuscular, transdermal, topical, subcutaneous routes, and inhalation. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. Other examples of drug formulations can be found in Liberman, H. A. and Lachman, L., Eds., Pharmaceutical Dosage Forms, Marcel Decker, New York, N.Y., 1980. The formulation of each substance or of the combination of two substances will generally include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

An effective dose can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication. For administration to animal or human subjects, the appropriate dosage of the a compound described above often is 0.01 to 1500 mg/kg, and sometimes 0.1 to 10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

Typical systemic dosages for all of the herein described conditions are those ranging from 0.01 mg/kg to 1500 mg/kg of body weight per day as a single daily dose or divided daily doses. Preferred dosages for the described conditions range from 0.5 to 1500 mg per day. A more particularly preferred dosage for the desired conditions ranges from 5 to 750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50, 25 to 50, 25 to 75, 25 to 100, 100 to 150, or 150 or more mg/kg/day, as a single daily dose or divided daily doses. In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound.

The compounds are conveniently administered in units of any suitable dosage form, including but not limited to one containing from about 7 to 3000 mg, from about 70 to 1400 mg, or from about 25 to 1000 mg of active ingredient per unit dosage form. For example, an oral dosage of from about 50 to 1000 mg is usually convenient, including in one or multiple dosage forms of 50, 100, 200, 250, 300, 400, 500, 600, 700, 800, 900 or 1000 mgs. Lower dosages may be preferable, for example, from about 10 to 100 or 1 to 50 mgs. Also contemplated are doses of 0.1-50 mg, 0.1-20 mgs., or 0.1-10 mgs. Furthermore, lower doses may be utilized in the case of administration by a non-oral route, as for example, by injection or inhalation.

The compound is administered for a sufficient time period to alleviate the undesired symptoms and the clinical signs associated with the condition being treated.

The active compound is included in the pharmaceutically acceptable carrier or diluent in an amount sufficient to deliver to a patient a therapeutic amount of compound in vivo in the absence of serious toxic effects. Pharmaceutically acceptable carriers that may be used in these pharmaceutical compositions are generally known in the art. They include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, solvents, salts or electrolytes, such as protamine sulfate, disodium hydrogen phosphate, potassium hydrogen phosphate, sodium chloride, zinc salts, silicates, colloidal silica, magnesium trisilicate, polyvinyl pyrrolidone, cellulose-based substances, polyethylene glycol, sodium carboxymethylcellulose, polyacrylates, waxes, oils, polyethylene-polyoxypropylene-block polymers, polyethylene glycol and wool fat. Pharmaceutically accepted vehicles can contain mixtures of more than one excipient in which the components and the ratios can be selected to optimize desired characteristics of the formulation including but not limited to shelf-life, stability, drug load, site of delivery, dissolution rate, self-emulsification, control of release rate and site of release, and metabolism.

Formulations can be prepared by a variety of techniques known in the art. Examples of formulation techniques can be found in literature publications and in texts such as “Water-insoluble drug formulation”, edited by Rong Liu, 2000, Interpharm Press.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

The compounds described herein, are particularly useful for the treatment or prevention of a disorder mediated by kinases or mediated by HSP90. In one embodiment, the compounds described herein, are useful for the treatment or prevention of a proliferative disorder, including cancer metastasis. In another embodiment, the compounds described herein, are useful for the treatment or prevention of an inflammatory or autoimmune disorder associated by kinases or HSP90.

An aspect of the invention relates to compounds and compositions that are useful for treating cancer.

Another aspect of the invention relates to the treatment of the following cancers: breast, ovary, cervix, prostate, testis, genitourinary tract, esophagus, larynx, glioblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia.

Another aspect of the invention is a method for treating cancer comprising administering an effective amount of a compound of the present invention to a patient with cancer.

Angiogenesis is characterized by the proliferation of endothelial cells to form new blood vessels (often called neovascularization). Inhibition of mitosis of endothelial cells results in inhibition of angiogenesis. Another aspect of this invention therefore relates to inhibition of undesirable mitosis, including undesirable angiogenesis. A mammalian disease characterized by undesirable cell mitosis, as defined herein, includes, but is not limited to, excessive or abnormal stimulation of endothelial cells (e.g., atherosclerosis), solid tumors and tumor metastasis, benign tumors, for example, hemangiomas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease).

Other undesired angiogenesis involves normal processes including ovulation and implantation of a blastula. The compositions described above can be used as a birth control agent by reducing or preventing uterine vascularization required for embryo implantation. Accordingly, the compositions described above can be used to block ovulation and implantation of a blastula or to block menstruation (induce amenorrhea).

Diseases associated with undesirable mitosis including neovascularization can be treated according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, and corneal graph rejection.

Other diseases associated with undesirable mitosis including neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

Another aspect of the invention relates to the treatment of inflammatory diseases including, but no limited to, excessive or abnormal stimulation of endothelial cells (e.g., atherosclerosis), solid tumors and tumor metastasis, benign tumors, for example, hemangiomas, acoustic neuromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease). Other undesired angiogenesis involves normal processes including ovulation and implantation of a blastula. Accordingly, the compositions described above can be used to block ovulation and implantation of a blastula or to block menstruation (induce amenorrhea).

Another aspect of this invention relates to a method of inhibiting HSP90 activity in a patient, comprising administering to a patient an effective amount of a compound of the present invention or a pharmaceutically acceptable salt or prodrug thereof. The invention also provides a method for treating a disease that is mediated by HSP90.

Another aspect of this invention relates to a method of inhibiting Aurora A activity in a patient, comprising administering to a patient an effective amount of a compound of the present invention or a pharmaceutically acceptable salt or prodrug thereof.

Another aspect of this invention relates to a method of treating or preventing a GSK-3-mediated disease with a GSK-3 inhibitor, comprising administering to a patient an effective amount of a compound of the present invention or a pharmaceutically acceptable salt or prodrug thereof.

One aspect of this invention relates to a method of enhancing glycogen synthesis and/or lowering blood levels of glucose in a patient in need thereof, which method comprises administering to the patient a therapeutically effective amount of a compound of the present invention or a pharmaceutical composition thereof. This method is especially useful for diabetic patients. Another method relates to inhibiting the production of hyperphosphorylated Tau protein, which is useful in halting or slowing the progression of Alzheimer's disease. Another method relates to inhibiting the phosphorylation of β-catenin, which is useful for treating schizophrenia.

Another aspect of the invention relates to inhibiting GSK-3 activity in a biological sample, which method comprises contacting the biological sample with a GSK-3 inhibitor of formula I, II, III, IVa or IVb.

Another aspect of this invention relates to a method of inhibiting GSK-3 activity in a patient comprising administering to the patient a compound of the present invention or a composition comprising said compound.

Another aspect of this invention relates to a method of treating or preventing a CDK-2-mediated disease comprising administering to a patient in need of such a treatment a therapeutically effective amount of a compound of the present invention or a pharmaceutical composition thereof.

Another aspect of the invention relates to inhibiting CDK-2 activity in a biological sample or a patient, which method comprises administering to the patient a compound of the present invention, or a composition comprising said compound.

Another aspect of this invention relates to a method of treating or preventing an ERK-2-mediated diseases comprising administering to a patient in need of such a treatment a therapeutically effective amount of a compound of the present invention or a pharmaceutical composition thereof.

Another aspect of the invention relates to inhibiting ERK-2 activity in a biological sample or a patient, which method comprises administering to the patient a compound of the present invention, or a composition comprising said compound.

Another aspect of this invention relates to a method of treating or preventing an AKT-mediated diseases comprising administering to a patient in need of such a treatment a therapeutically effective amount of a compound of the present invention or a pharmaceutical composition thereof.

Another aspect of the invention relates to inhibiting AKT activity in a biological sample or a patient, which method comprises administering to the patient a compound of the present invention, or a composition comprising said compound.

Another aspect of this invention relates to a method of treating or preventing a Src-mediated disease comprising administering to a patient in need of such a treatment a therapeutically effective amount of a compound of the present invention or a pharmaceutical composition thereof.

Another aspect of the invention relates to inhibiting Src activity in a biological sample or a patient, which method comprises administering to the patient a compound of the present invention, or a composition comprising said compound.

Another aspect of this invention relates to a method of treating or preventing an Lck-mediated disease with an Lck inhibitor, which method comprises administering to a patient in need of such a treatment a therapeutically effective amount of a compound of the present invention, or a pharmaceutical composition thereof.

Another aspect of the invention relates to inhibiting Lck activity in a biological sample or a patient, which method comprises administering to the patient a compound of the present invention, or a composition comprising said compound.

Another aspect of this invention relates to a method of treating or preventing an Abl-mediated disease with an Abl inhibitor, which method comprises administering to a patient in need of such a treatment a therapeutically effective amount of a compound of the present invention, or a pharmaceutical composition thereof.

Another aspect of the invention relates to inhibiting Abl activity in a biological sample or a patient, which method comprises administering to the patient a compound of the present invention, or a composition comprising said compound.

Another aspect of this invention relates to a method of treating or preventing a cKit-mediated disease comprising administering to a patient in need of such a treatment a therapeutically effective amount of a compound of the present invention, or a pharmaceutical composition thereof.

Another aspect of the invention relates to inhibiting cKit activity in a biological sample or a patient, which method comprises administering to the patient a compound of the present invention, or a composition comprising said compound.

Another aspect of this invention relates to a method of treating or preventing a Flt3-mediated disease comprising administering to a patient in need of such a treatment a therapeutically effective amount of a compound of the present invention, or a pharmaceutical composition thereof.

Another aspect of the invention relates to inhibiting Flt3 activity in a biological sample or a patient, which method comprises administering to the patient a compound of the present invention, or a composition comprising said compound.

Another aspect of this invention relates to a method of treating or preventing a KDR-mediated disease comprising administering to a patient in need of such a treatment a therapeutically effective amount of a compound of the present invention, or a pharmaceutical composition thereof.

Another aspect of the invention relates to inhibiting KDR activity in a biological sample or a patient, which method comprises administering to the patient a compound of the present invention, or a composition comprising said compound.

An amount effective to inhibit protein kinase, is an amount that causes measurable inhibition of the kinase activity when compared to the activity of the enzyme in the absence of an inhibitor. Any method may be used to determine inhibition, such as, for example, the Biological Testing Examples described below.

The compounds of the invention as described above can be synthesized using methods, techniques, and materials known to those of skill in the art, such as described, for example, in March, ADVANCED ORGANIC CHEMISTRY 4.sup.th Ed., (Wiley 1992); Carey and Sundberg, ADVANCED ORGANIC CHEMISTRY 3.sup.rd Ed., Vols. A and B (Plenum 1992), and Green and Wuts, PROTECTIVE GROUPS IN ORGANIC SYNTHESIS 2.sup.nd Ed. (Wiley 1991). Starting materials useful for preparing compounds of the invention and intermediates thereof are commercially available from sources, such as Aldrich Chemical Co. (Milwaukee, Wis.), Sigma Chemical Co. (St. Louis, Mo.), Maybridge (Cornwall, England), Asinex (Winston-Salem, N.C.), ChemBridge (San Diego, Calif.), ChemDiv (San Diego, Calif.), SPECS (Delft, The Netherlands), Timtec (Newark, Del.), or alternatively can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the present compounds and/or starting materials thereof are either described in the art or will be readily apparent to the skilled artisan. Alternatives to the reagents and/or protecting groups may be found in the references provided above and in other compendiums well known to the skilled artisan.

Preparation of the present compounds may include one or more steps of protection and deprotection (e.g., the formation and removal of acetal groups). Guidance for selecting suitable protecting groups can be found, for example, in Greene & Wuts, “Protective Groups in Organic Synthesis,” Wiley Interscience, 1999. In addition, the preparation may include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. The preparation may also involve any other methods of protection and deprotection, purification and identification and quantification that are well known in the chemical arts.

Synthesis of the examples of the present compounds are illustrated in the general and specific schemes below. One skilled in the art can readily derive the synthesis of the present compounds from the following examples according to the methods discussed above.

### Preparation of Library of Pochonin Oxime Analogs:

In some embodiments, the synthetic planning of the library was based on previously developed chemistry^{[27]} and leveraged on the use of solid phase synthesis and polymer-bound reagents. To broaden the SAR of the pochoximes, a library with four points of diversity was envisioned (see 1, Scheme 2) which would stem from a divergent coupling of fragment A to B followed by oxime formation and introduction of fragment C and then D. The choice of fragments A to D was based on preliminary structure-activity data of the present inventors^{[27-29]} and the objective of biasing the conformational profile of the macrocycle through different ring sizes and additional small substituents. Regarding the aryl moiety (fragment A), preliminary experiments had shown that modifications of either phenol was detrimental; however, the presence or absence of a chlorine at R¹ did have a subtle impact on the activity and both alternatives were included in the library. Regarding the lower part of the macrocycle (derived from fragment B), the present inventors^{[27]} and others {Barluenga, 2008 #39}^{[30]} had previously found that the saturated fragment B2 was tolerated however, additional substitutions had not been investigated and three new combinations (B3-5) were included. Regarding the top part of the macrocycle (fragment C), eight modifications were considered leading to lactones (C1-2, C4) or lactame (C3), different macrocycle size (C5-6) and an alkyne (C8) which would afford a diene as the product of the metathesis rather than the usual endocyclic alkene. The last group (D) included the largest diversity to probe the optimal oxime substituent.^{[27, 31, 32]}

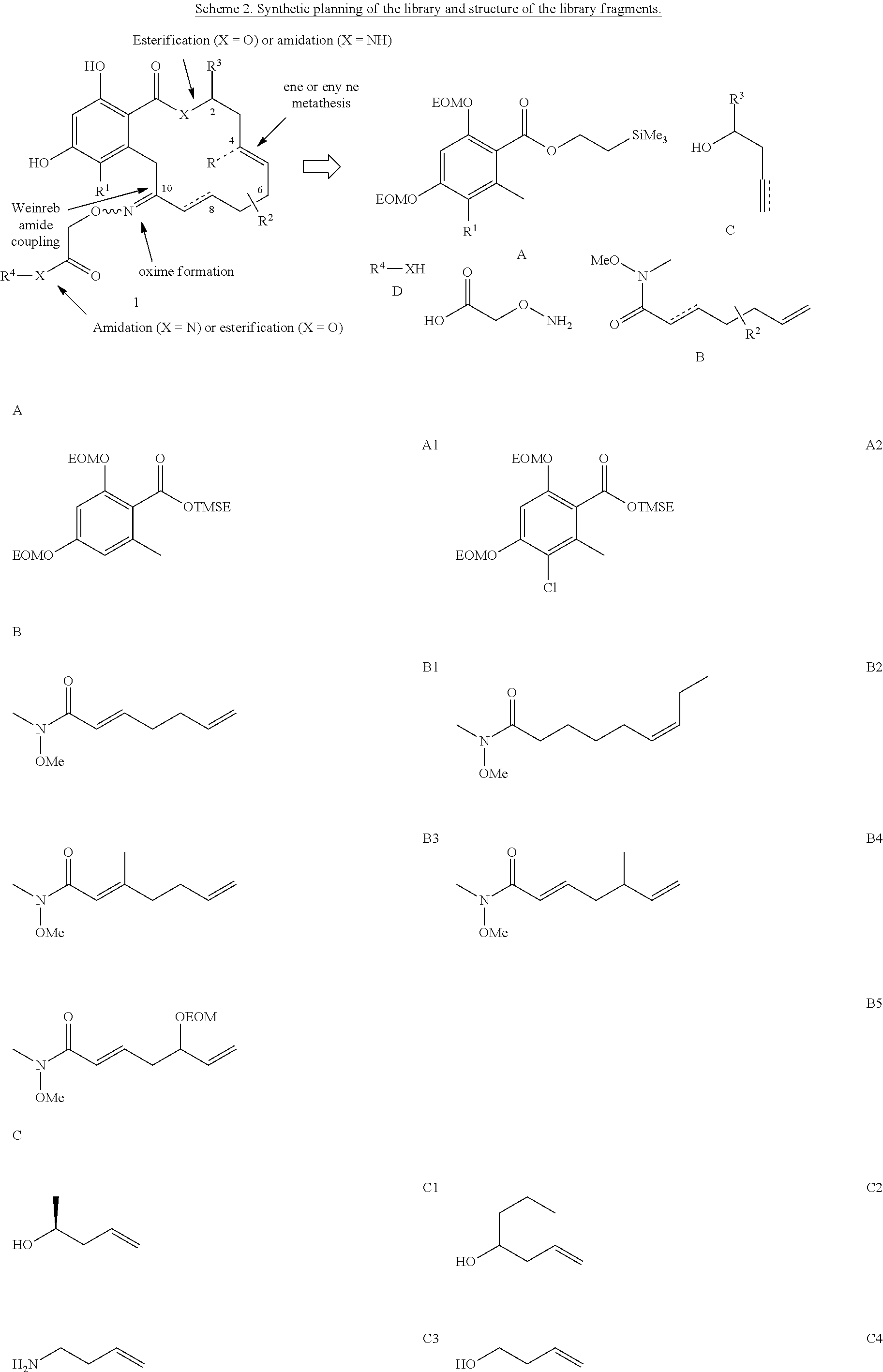

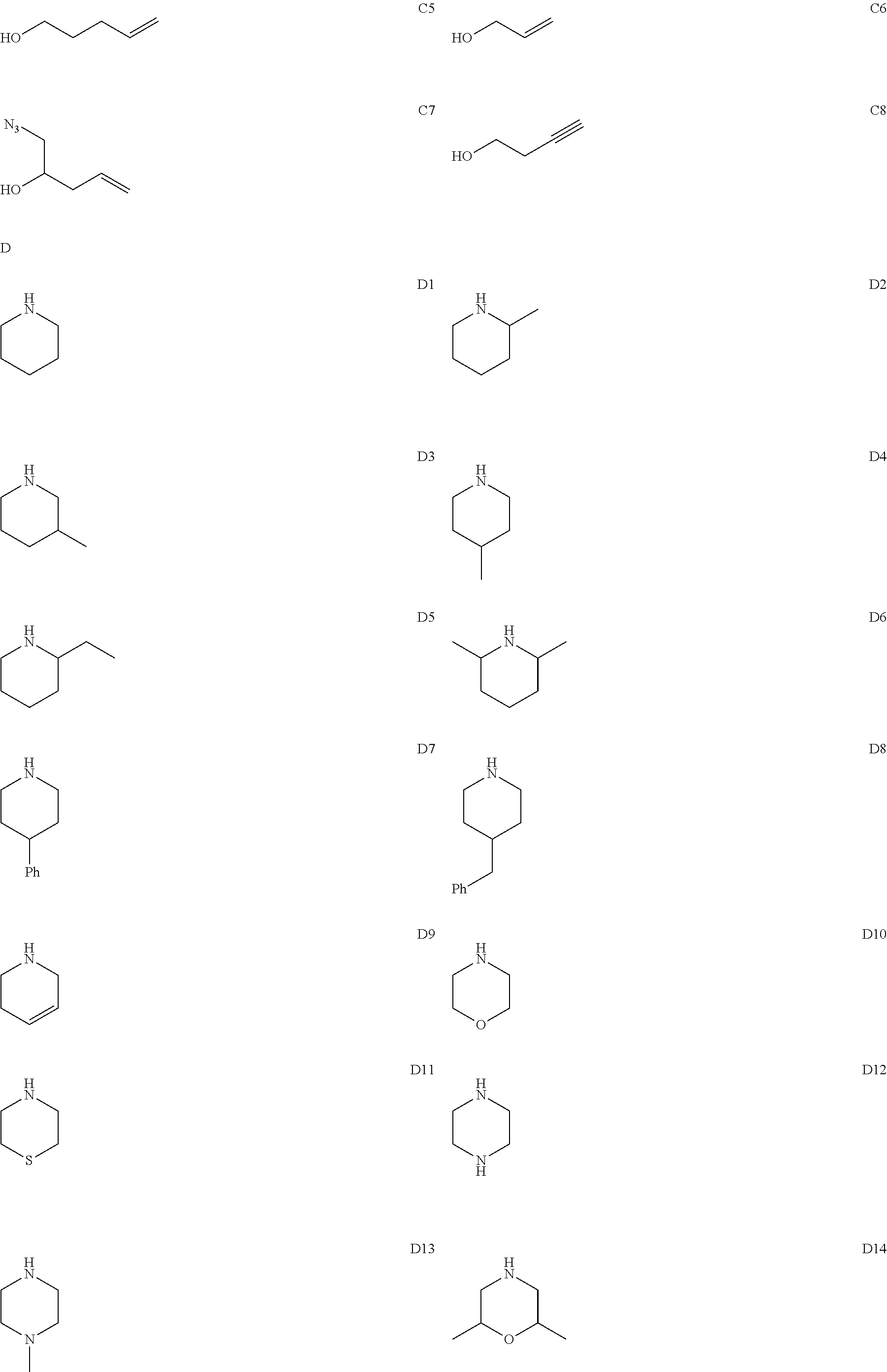

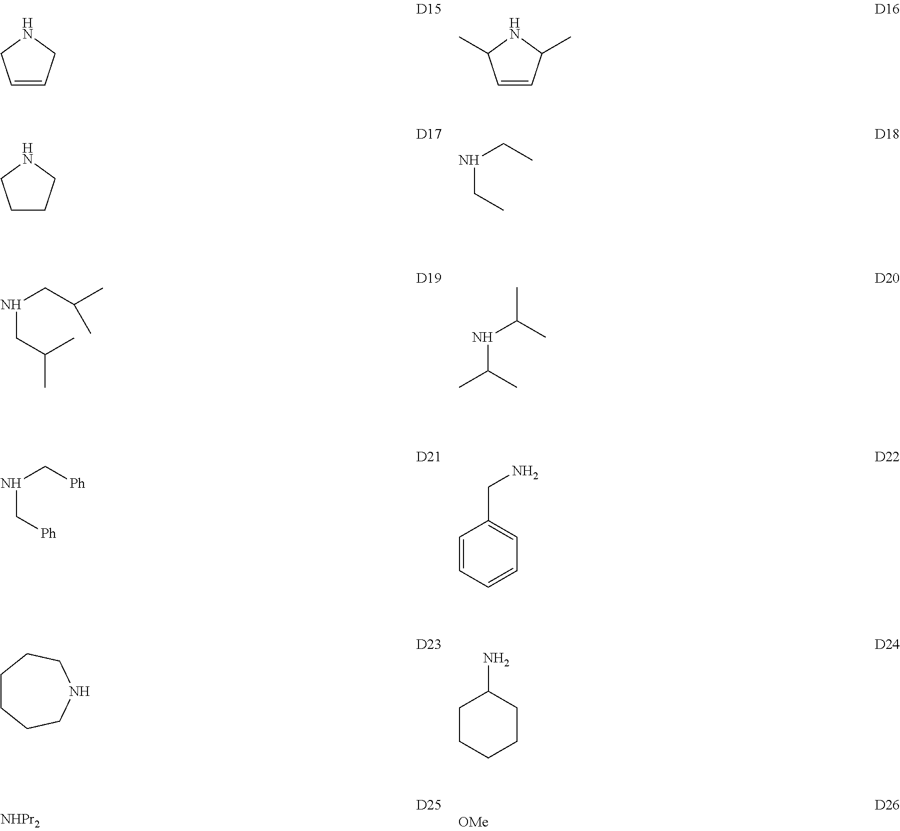

As shown in Scheme 3, deprotonation of the toluate fragment A with LDA followed by the addition of fragment B afforded a total of ten different combinations of intermediate 2 in moderate to good yield (50-85%) except for the coupling with fragment B2 which afforded unacceptable yield (<10%) presumably to competing enolization of the Weinreb amide. Alternatively, the same product could be obtained by conjugate reduction of the coupling product obtained with fragment B1 using NaCNBH₃ (25-50%). Each product was treated with aminooxyacetic acid and, after filtration through a pad of silica to remove excess aminooxyacetic acid, the resulting oximes 2 were loaded on a chlorotrityl resin to afford polymer-bound intermediates 3. To ensure a complete consumption of the starting material, a high loading resin (1.1 mmol/g) was used in excess and once all starting material had been consumed (24 h), the resin was capped with the addition of AcOH to afford ten resins of 3. The 2-(trimethylsilyl)ethyl ester was then cleaved under the action of TBAF to afford 4 and each batch of resin was further divided in eight batches for the esterification or amide formation with fragments C. It was found to be essential to thoroughly wash the resin with a 1% AcOH solution in CH₂Cl₂ after the TBAF deprotection to protonate the polymer-bound carboxylate and remove tetrabutyl ammonium salts. Each resin was then subjected to a metathesis reaction with the second generation Grubbs catalyst^{[33]} under microwave irradiation until 120° C. for 45 min to afford the desired polymer-bound macrocycles 5. To ensure completion of the reaction and equilibration to the thermodynamically more favorable E-alkene, the reactions were repeated three times with a catalyst loading of 6% in each treatment. For the ene-yne metathesis (resins including fragment C8), an additional alkene was added in solution to facilitate catalyst turnover. The library of macrocycles 5 was then cleaved from the resin using hexafluoro isopropanol (HFIP) which was found to preserve the integrity of the EOM groups to afford, after purification, the products in 20-30% yield over five steps. The macrocycles bearing a free carboxylic acid were then further divided in separate pools for coupling to fragments D using polymer-bound carbodiimide and 4-DMAP with an excess of amine (>2.0 equiv) which afforded the products with excellent conversion (>90%). The excess of amine was removed during the evaporation or sequestered along with DMAP during the subsequent treatment with a large excess (10 equiv) of sulfonic acid resin in MeOH to perform the EOM deprotection (>75% isolated yield for two steps). While not all permutations were pursued, a library containing at least one example of each fragment was prepared. All compounds were purified by PTLC however no effort was made to separate the E and Z isomers of the oxime which typically have very similar if not identical R_{f}. The purity of all products was assessed by LC/MS and a sample of the library was analyzed by NMR. Generally, compounds were obtained as a 1:1 E:Z mixture of oxime geometry.

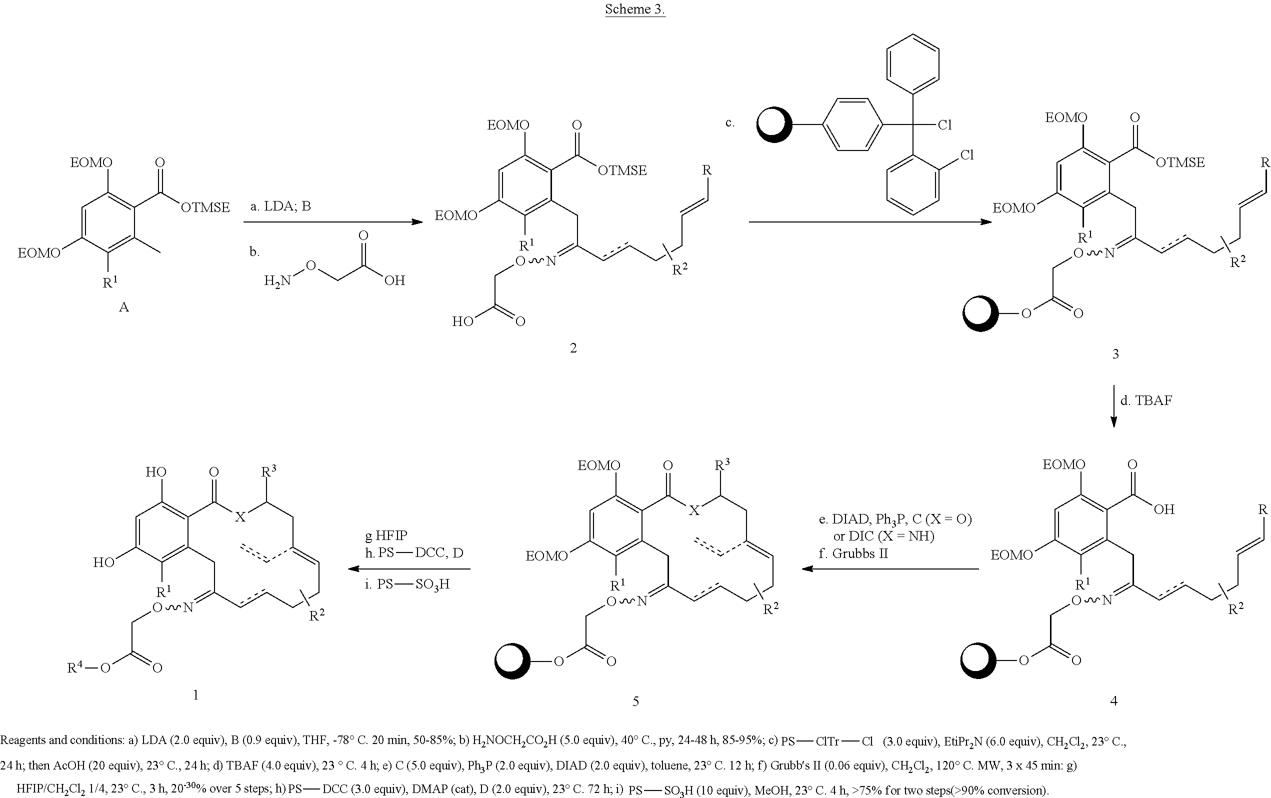

A subset of this library was screened for its affinity to human HSP90α using a competition assay with a fluorescein labeled analog of 17AAG^{[34]} and in a cellular assay using Her-2 as a pharmacodynamic marker of cellular efficacy.^{[35]} The present inventors had previously observed that the Z isomers were less potent than the E isomers (particularly in cellular assays), however, as the E/Z ratio is consistent within the library, the results obtained should be qualitatively significant. The results are summarized in Table 1 below and revealed interesting opportunities regarding modifications which are well tolerate as a single point modification or in conjunction with other modifications. As previously noted, there are only moderate differences in activity for compounds with the unsubstituted aryl ring (A1) and chlorinated aryl ring (A2) (entry 1 vs entry 41). However, this can become significant in conjunction with other modifications. For example, the presence of the chlorine atom is beneficial in conjugation with fragment C2 where the combination A2C2 is roughly five times more potent in its affinity for HSP90 than A1C2 (entries 3 vs 42, 30 vs 47 and 34 vs 49). On the other hand, it is detrimental in conjunction with B4 (entries 36 vs 50 and 38 vs 52). Regarding the lower part of the macrocycles, an α,β-conjugated oxime is systematically better than the saturated one (B1 vs B2). An additional methylene at the β position (B3) or γ position (B4) as well as a hydroxyl group at the γ position (B5) are generally well tolerated with the combinations A1B3C4D1 (entry 35), A1B4C4D1 (entry 38) and A1B5C1D1 (entry 40) being amongst the fittest ligand from the library. Regarding the upper part of the macrocycle, there was generally little difference between the activity of compounds having the chiral methyl group (C1) or a simple primary ester (C4). A longer alkyl chain at carbon 2 (C2 and C7) were tolerated in some permutations (entries 42 and 45) but not with fragment B4 (entry 51). Modification of the lactone for a lactame (C3) led to a significant decrease in affinity (entry 43 vs 41) and different macrocycle size (13-member ring with C5 and 15-member ring with C6) also led to reduced affinity. Fragment C8 which affords a product substituted at carbon 4 with a vinyl group also led to a significant decrease in affinity (entry 46). The present inventors had already noted that aliphatic amide provided the best activity for fragment D however, this larger panel of amides refined the structure-activity relationship. Substituents on the piperidine ring is well tolerated at the β (D3) and δ (D4) position but not at the α position (D2, D5, D6) nor with too large substituent (D7 and D8). Five- (D15-17) or seven-member rings (D23) as well as acyclic secondary amides (D18-21, D25) lead to reduced affinity. One modification did provide a slight improvement in affinity: dehydropiperidine D9 (entry 11).

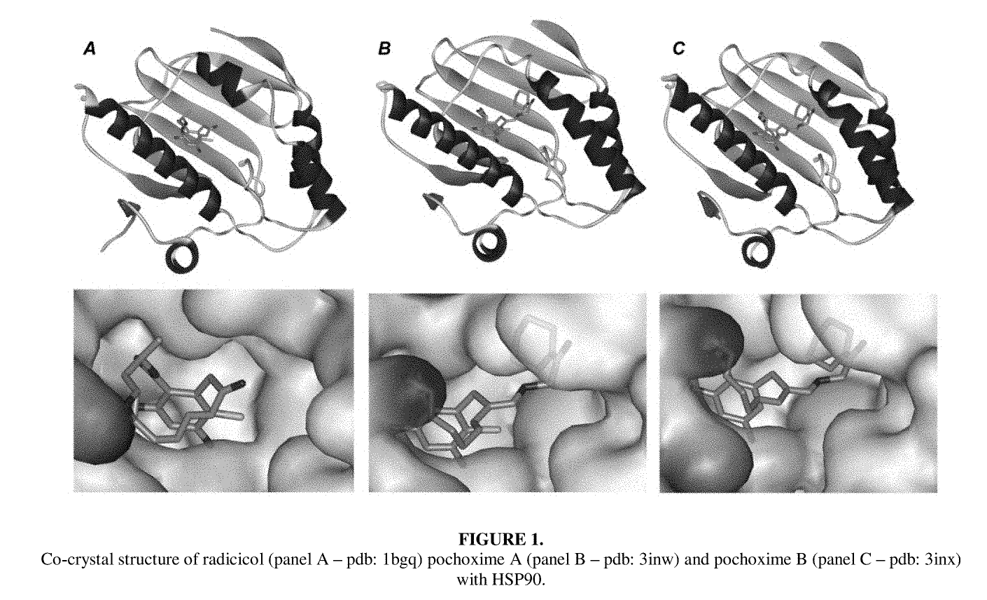

The co-crystal structure of the N-terminal part HSP90 bound to radicicol (1bgq, FIG. 1, panels A)^{[36]} is very similar to the structure of apo-HSP90(1yer)^{[37]}. Crystal structures have also been reported for the functionally related ER chaperone GRP94 (1u0z)^{[38]} again showing a similar conformation of the ATP-binding pocket of HSP90. Likewise, the co-crystal structure of several resorcylide analogs of radicicol with HSP90 have also been reported to bind to a similar conformation of HSP90.^{[30]} The studies of the present inventors on the binding mode of pochoximes using docking, binding free energy calculations and NMR ¹⁵N chemical shifts with yeast HSP90 suggested a similar binding mode to radicicol.^{[39]} However, they did not provide a rationale for the significant enhancement of activity from pochonin D to the pochoximes. While the ketone of pochonin D does point towards a small unoccupied pocket, docking experiments suggested that its conversion to oximes with large substituents such as the piperidine amide present in pochoximes A, B, and C would be too large to fit and placed outside of the binding pocket. Cocrystalyzation of pochoxime A and B using a twofold molar excess of ligand with the N-terminal domain of human HSP90α afforded crystals which diffracted at 1.95 Å (pdb ID: 3inw) and 1.75 Å (pdb ID: 3inx) respectively (see supplemental information for statistical analysis). In both cases, the inhibitor could be placed unambiguously in the calculated electron densities. Analysis of both structures revealed a significant rearrangement in the “ATP-lid” with the peptide segment from Ile104 through Ala124 assuming a contiguous α-helix (FIG. 1, panels B and C). This rearrangement creates a large lyophilic pocket at the interface of the side chains of Met98, Leu103, Phe138 and Trp162 with the piperidine moiety sandwiched between the aryl moiety of Thp162 and the side chains of Met98. The rearrangement thus creates the opportunity for favorable interactions with the oxime substituent and hence, a rational for the enhancements in activity of the pochoximes as well as the preference of lyophilic groups on the hydroxylamine such a piperidine amides. Based on the similarity between the binding mode of pochonin and radicicol, extrapolation of these results to radicicol can also account for the benefits of oximes.^{[31, 32]} A similar change in HSP90 conformation has been noted for some purine-based inhibitor PU3 (pdb ID luym),^{[37]} however, in the later case, the pocket created by Met98, Leu103 and Thp162 is less open and only accommodates a methoxy group rather than the whole piperidine ring while the aryl ring of Ph138 is involved in n-stacking interactions to the benzyl moiety of PU3. Likewise, the cocrystal structure of a representative 2-aminobenzamide (pdb ID: 3d0b)^{[40]} was shown to have a contiguous α-helix in the “ATP-lid” however, it does not exploit lypophylic interactions between Met98 and Trp162.

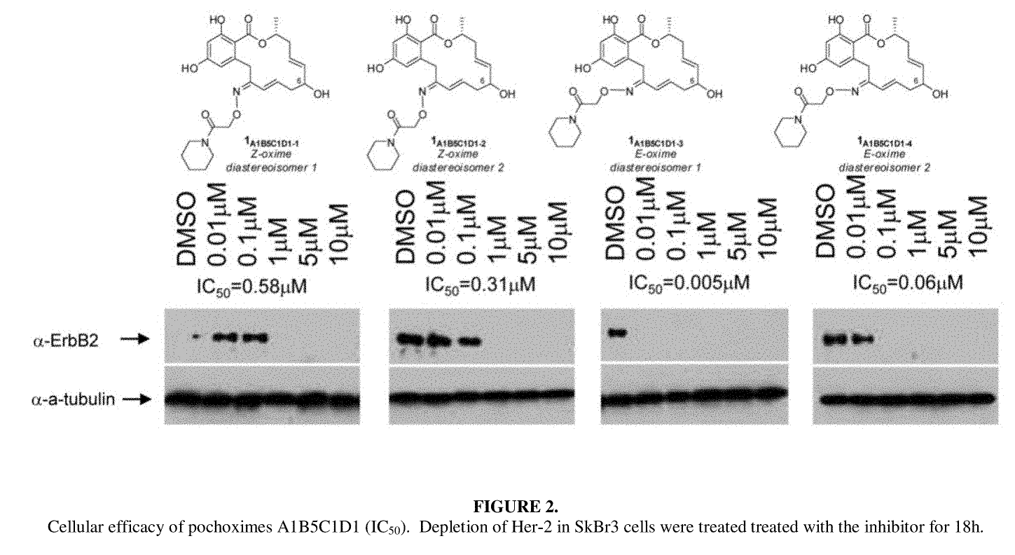

Based on this structure, substitution at the allylic position such as in compounds which include fragment B4 and B5 should point towards the solvent. Compound 1_{A1B5C1D1} bearing a hydroxyl substitution at the allylic position (carbon 6) was deemed most interesting as it should improve aqueous solubility relatively to pochoximes A, B, and C and provide a handle to label the inhibitor with a marker or affinity tag. This compound was prepared as a mixture of four diastereoisomers (two oxime geometries with either stereochemistry at carbon 6) which proved to be separable by HPLC. Interestingly, while the E and Z oximes were readily identifiable by NMR analysis, the diastereoisomers stemming from the different chirality of the allylic hydroxyl group had very similar NMR and its stereochemistry could not be inferred. The four isomers were tested for their cellular efficacy (FIG. 2). As previously noted, the E isomers of the oximes were considerably more potent than the Z oximes (60 to 100 fold). There was also a notable difference in activity (ca. 10 fold) between the two different diastereoisomers with the most potent compound being active at 5 nM making it the most potent pochoxime to date (tentatively assigned as the R isomer based on docking).

In order to prepare derivatives of this newly identified analog, compound 6 bearing a silyl protecting group on the allylic hydroxyl group was prepared according to a previously developed protocol (see supplemental information for detailed synthetic protocols).^{[29]} As shown in scheme 4, selective silyl deprotection of 6 using TBAF afforded 7 onto which was introduce a short linker via alkylation, azide displacement and reduction to afford amine 9. Labeling of 9 with Cy3 followed by EOM deprotection afforded pochoxime-Cy3 conjugate 10. Alternatively, compound 7 was labeled with biotin via a four step sequence involving coupling to a short PEG linker, Fmoc deprotection, coupling to biotin and global deprotection with TFA. It should be noted that in this case, global deprotection with sulfonic acid resin led to a trans-esterification of the PEG-ester. The affinity of these derivatives for human HSP90α was found to be below 30 nM suggesting the linker to a fluorophore or biotin in the form of an ether or ester does not perturb the binding.

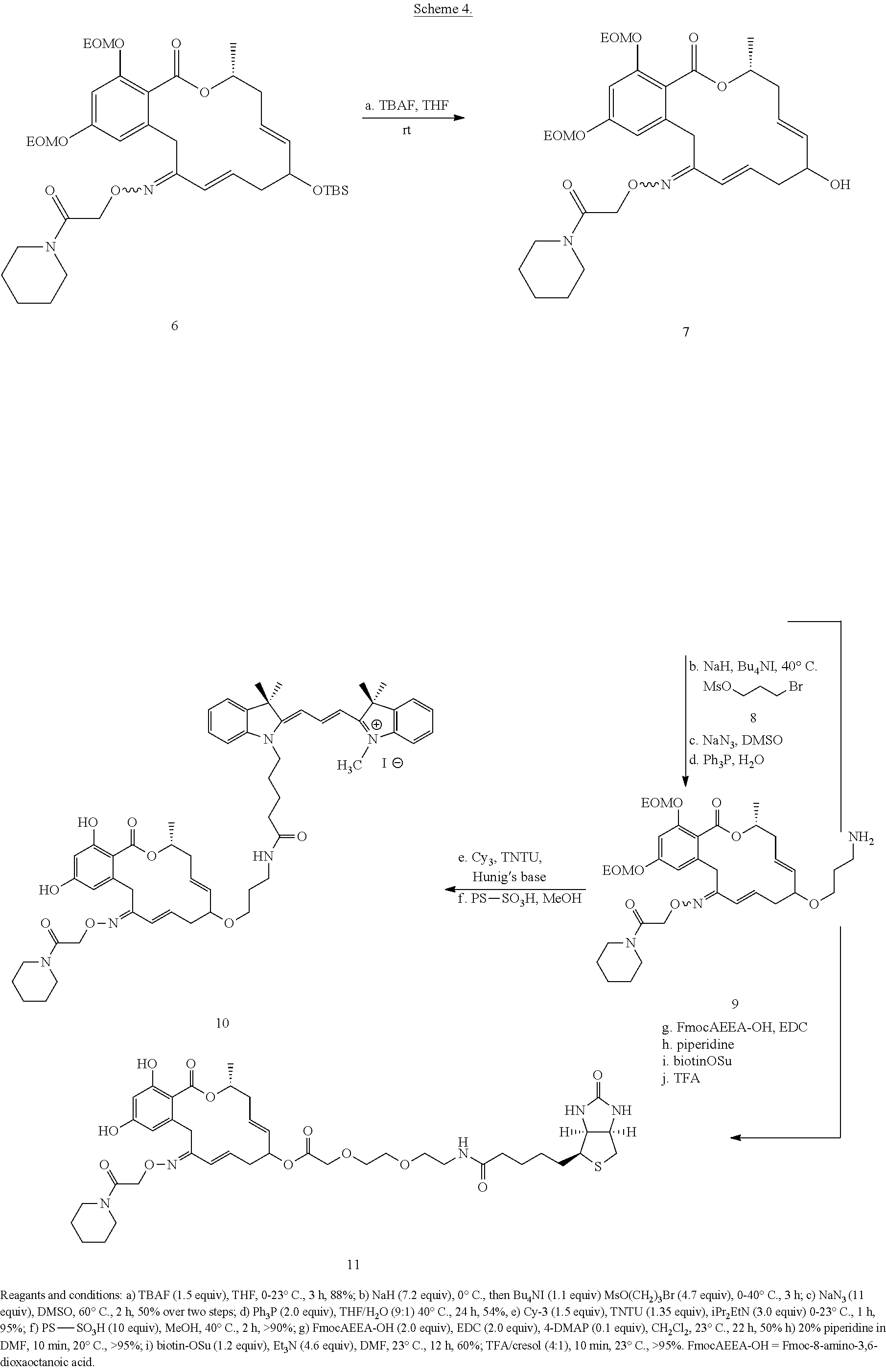

This extended library of pochoximes led to the identification of several analogs with enhanced activity. Introduction of a hydroxyl group at carbon 6 on the macrocycle afforded the most potent pochoxime to date showing cellular efficacy below 10 nM. The co-crystal structure of pochoxime A and B with human HSP90α shows that the pochoximes bind to a different conformation of HSP90 than the closely related radicicol. Whether this alternative binding mode translates to different biological activities is currently under investigation. The identification of derivatives with affinity tag or marker should assist in this endeavour and could prove useful in diagnostics and imaging. Finally, the flexibility in the “ATP-lid” region^{[41]} of human HSP90 as exemplified by the structures reported herein should be an important consideration in the design of inhibitors. Furthermore, differences in the flexibility in this region of the protein amongst the different paralogs of HSPs or HSPs from different species may be exploited to achieve selective inhibition.

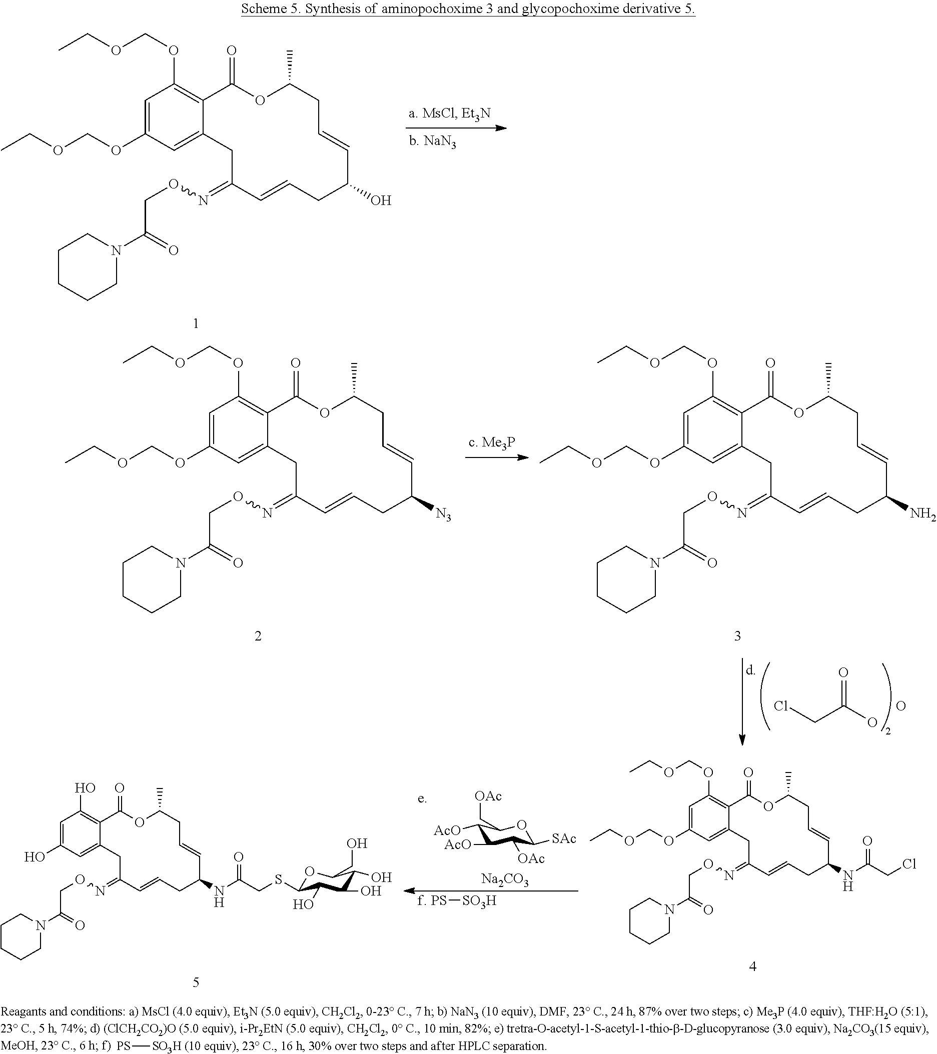

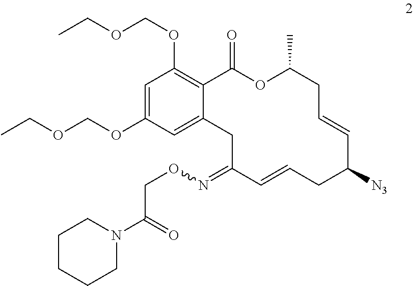 [SMILES: CCOCOc1cc2c(c(OCOCC)c1)C(=O)O[C@H](C)C/C=C/[C@@H](N=[N+]=[N-])C/C=C/C(=NOCC(=O)N1CCCCC1)C2]

### Preparation of Azide 2.

Alcohol 1 (1.0 equiv, 100 mg, 0.17 mmol) was dissolved in dichloromethane (5.0 mL) and the solution cooled to 0° C. Triethylamine (5.0 equiv, 100 μL, 0.85 mmol) and methanesulphonyl chloride (4.0 equiv, 52 μL, 0.68 mmol) were added slowly at 0° C. The reaction mixture was stirred for 7 hours at 23° C. and then quenched with sat. aqueous NaHCO₃. The extracted organic phase was washed with brine, dried over Na₂SO₄, filtered and the solvent was concentrated under reduced pressure to leave a yellow residue. The crude mesylate was dissolved in DMF (5.0 mL), sodium azide (10 equiv, 111 mg, 1.7 mmol) was added and stirred at 23° C. After 24 hours the reaction mixture was filtered and concentrated under reduced pressure. The residue was purified by flash chromatography (SiO₂, 1:1 EtOAc/Hexane), to afford azide 2 (87 mg, 87% over two steps) as a colourless oil. R_{f}=0.3 (1:1 EtOAc/Hexane); ¹H NMR (CDCl₃, 400 MHz) δ=6.87 (d, J=16.0 Hz, 1H), 6.74-6.73 (m, 2H), 6.66 (d, J=2.0 Hz, 1H), 6.60 (d, J=2.0 Hz, 1H), 6.15 (d, J=16.4 Hz, 1H), 6.04-5.92 (m, 2H), 5.75-5.69 (m, 2H), 5.42 (dt, J=15.6, 4.4 Hz, 2H), 5.24-5.19 (m, 10H), 4.84 (d, J=3.2 Hz, 4H), 4.26 (d, J=15.2 Hz, 1H), 4.18-4.17 (m, 2H), 3.71-3.68 (m, 8H), 3.61-3.58 (m, 6H), 3.48-3.42 (m, 6H), 2.53-2.49 (m, 2H), 2.42-2.34 (m, 5H), 1.71-1.59 (m, 12H), 1.45 (dd, J=6.0, 1.6 Hz, 6H), 1.30-1.20 (m, 12H) ppm; HRMS (MALDI-TOF) m/z [M+H]⁺ calcd for C₃₁H₄₄N₅O₈: 614.3112. found: 614.3139.

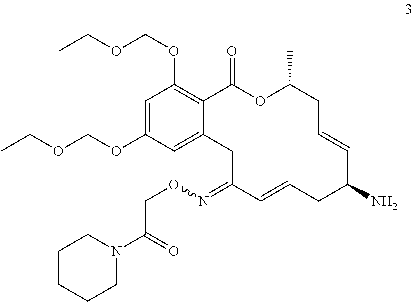 [SMILES: CCOCOc1cc2c(c(OCOCC)c1)C(=O)O[C@H](C)C/C=C/[C@@H](N)C/C=C/C(=NOCC(=O)N1CCCCC1)C2]

### Synthesis of Amine 3.

To a solution of azide 2 (1.0 equiv, 83.6 mg, 0.136 mmol) in a mixture of THF: H₂O (5.5 mL, 5:1) was added Me₃P (4.0 equiv, 0.54 mL, 1M in THF). The reaction mixture was stirred at 23° C. for 5 hours and then concentrated under reduced pressure. The compound was then purified by reverse phase to yield amine 3 in a 74% yield (59 mg, 100 mmol) as a mixture of isomers. HRMS (MALDI-TOF) m/z [M+H]⁺ calcd for C₃₁H₄₆N₃O₈: 588.3207. found: 588.3212.

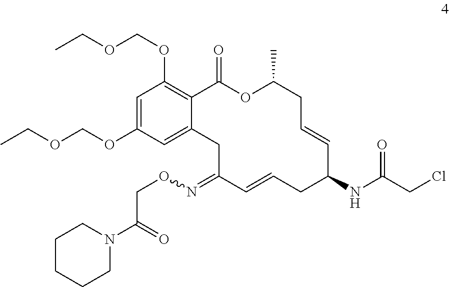 [SMILES: CCOCOc1cc2c(c(OCOCC)c1)C(=O)O[C@H](C)C/C=C/[C@@H](NC(=O)CCl)C/C=C/C(=NOCC(=O)N1CCCCC1)C2]

### Synthesis of Chloride 4.

Amine 3 (59 mg, 100 μmol) was dissolved in dichloromethane (5.0 mL) and cooled to 0° C., i-Pr₂EtN (5.0 equiv, 88 μL, 500 μmol) and chloroaceticanhydride (5.0 equiv, 84 mg, 500 μmol) were added sequentially and stirred for 10 min. The reaction mixture was quenched with aqueous NaHCO₃ and washed with brine, dried and evaporated in vacuo. The residue was purified by flash chromatography (SiO₂, 6:4 EtOAc/Hexane), to give chloride 4 (55 mg, 82%). R_{f}=0.15 (1:1 EtOAc/Hexane); ¹H NMR (CDCl₃, 400 MHz) δ=6.82 (d, J=16.4 Hz, 1H), 6.65-6.64 (m, 2H), 6.56 (d, J=2.0 Hz, 1H), 6.48 (t, J=8.0 Hz, 2H), 6.12 (d, J=16.8 Hz, 1H), 5.96-5.80 (m, 2H), 5.43-5.42 (m, 1H), 5.41-5.40 (m, 1H), 5.23-5.21 (m, 2H), 5.15-5.09 (m, 10H), 4.75 (s, 4H), 4.58 (bs, 2H), 4.42 (d, J=15.2 Hz, 1H), 3.98 (d, J=2.0 Hz, 4H), 3.68-3.59 (m, 8H), 3.52-3.34 (m, 10H), 3.09 (d, J=15.2 Hz, 1H), 2.38-2.31 (m, 8H), 1.58-1.44 (m, 12H), 1.35 (d, J=6.0 Hz, 6H), 1.20-1.11 (m, 12H) ppm.

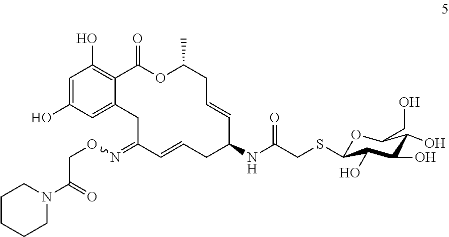

### Synthesis of Thio Glycoside 5.

Tetra-O-acetyl-1-S-acetyl-1-thio-β-D-glucopyranose (3.0 equiv, 82 mg, 0.201 mmol) was dissolved in methanol (2.0 mL). Sodium carbonate (15 equiv, 105 mg, 1.0 mmol) was added and stirred at 23° C. for 3 hours and then chloride 3 (1.0 equiv, 45 mg, 0.67 mmol) was added and further stirred for 3 hours. The reaction mixture was filtered, neutralized with Dowex 50WX2-100 resin and evaporated in vacuo. The crude product was dissolved in methanol (3.0 mL) and PS—SO₃H (10 equiv, 223 mg, 3.0 mmol/g) was added and stirred at 23° C. for 16 hours. The reaction mixture was filtered, and evaporated in vacuo. The oxime isomers were separated by HPLC [Agilent Zorbax Eclipse XDB-C18 9.4 mm×25 mm column; flow rate 3.0 mL/min; linear gradient from 80% to 40% H₂O (0.01% TFA) in acetonitrile (0.01% TFA)] to give thio glycosides 5 (Z-isomer: 6.0 mg; E-isomer: 7.9 mg).

Glycopochoxime 5 Z-isomer ¹H NMR (CD₃OD, 400 MHz) δ=6.81 (d, J=16 Hz, 1H), 6.33 (d, J=2.4 Hz, 1H), 6.20 (d, J=2.4 Hz, 1H), 6.19-6.11 (m, 1H), 5.72-5.64 (m, 1H), 5.49 (dd, J=15.2, 6 Hz, 1H), 5.36-5.29 (m, 1H), 4.48-4.46 (m, 1H), 4.43 (d, J=4.8 Hz, 1H), 4.02 (d, J=15.2 Hz, 1H), 3.88 (d, J=11.6 Hz, 1H), 3.68-3.61 (m, 4H), 3.60-3.55 (m, 4H), 3.31-3.28 (m, 4H), 3.25 (t, J=8.4 Hz, 1H), 2.64-2.59 (m, 1H), 2.50-2.40 (m, 2H), 2.30-2.23 (m, 1H), 1.73-1.62 (m, 6H), 1.47 (d, J=6.4 Hz, 3H) ppm; ¹³C NMR (CD₃OD, 100 MHz) δ 174.6, 173.5, 172.4, 164.8, 164.4, 159.4, 144.2, 141.6, 135.9, 130.5, 124.4, 113.1, 111.1, 104.8, 89.0, 84.8, 82.0, 76.9, 75.2, 75.1, 74.0, 65.5, 55.1, 49.7, 46.7, 41.9, 40.5, 38.1, 36.4, 30.0, 29.2, 27.9, 22.2 ppm; HRMS (MALDI-TOF) m/z [M+H]⁺ calcd for C₃₃H₄₆N₃O₁₂S: 708.2724. found: 708.2712.

Glycopochoxime 5 E-isomer ¹H NMR (CD₃OD, 400 MHz) δ=6.09-6.07 (m, 2H), 5.96-5.82 (m, 2H), 5.57-5.49 (m, 1H), 5.31 (dd, J=15.6, 6.4 Hz, 1H), 5.21-5.17 (m, 1H), 4.32-4.24 (m, 4H), 3.75 (d, J=11.2 Hz, 1H), 3.66 (d, J=15.6 Hz, 1H), 3.54-3.46 (m, 4H), 3.40-3.36 (m, 4H), 3.19-3.15 (m, 4H), 3.10 (t, J=9.2 Hz, 1H), 2.54-2.47 (m, 1H), 2.35-2.25 (m, 2H), 2.10-2.03 (m, 1H), 1.59-1.58 (m, 2H), 1.53-1.48 (m, 4H), 1.33 (d, J=6.4 Hz, 3H) ppm; ¹³C NMR (CD₃OD, 100 MHz) δ 170.5, 169.7, 168.1, 161.1, 158.1, 139.3, 133.5, 132.0, 127.4, 126.6, 108.1, 100.9, 85.0, 80.8, 78.1, 73.0, 71.4, 71.3, 70.1, 61.5, 51.4, 45.8, 42.8, 37.8, 36.4, 32.4, 29.0, 26.1, 25.3, 24.0, 18.2 ppm; HRMS (MALDI-TOF) m/z [M+H]⁺ calcd for C₃₃H₄₆N₃O₁₂S: 708.2724. found: 708.2738.

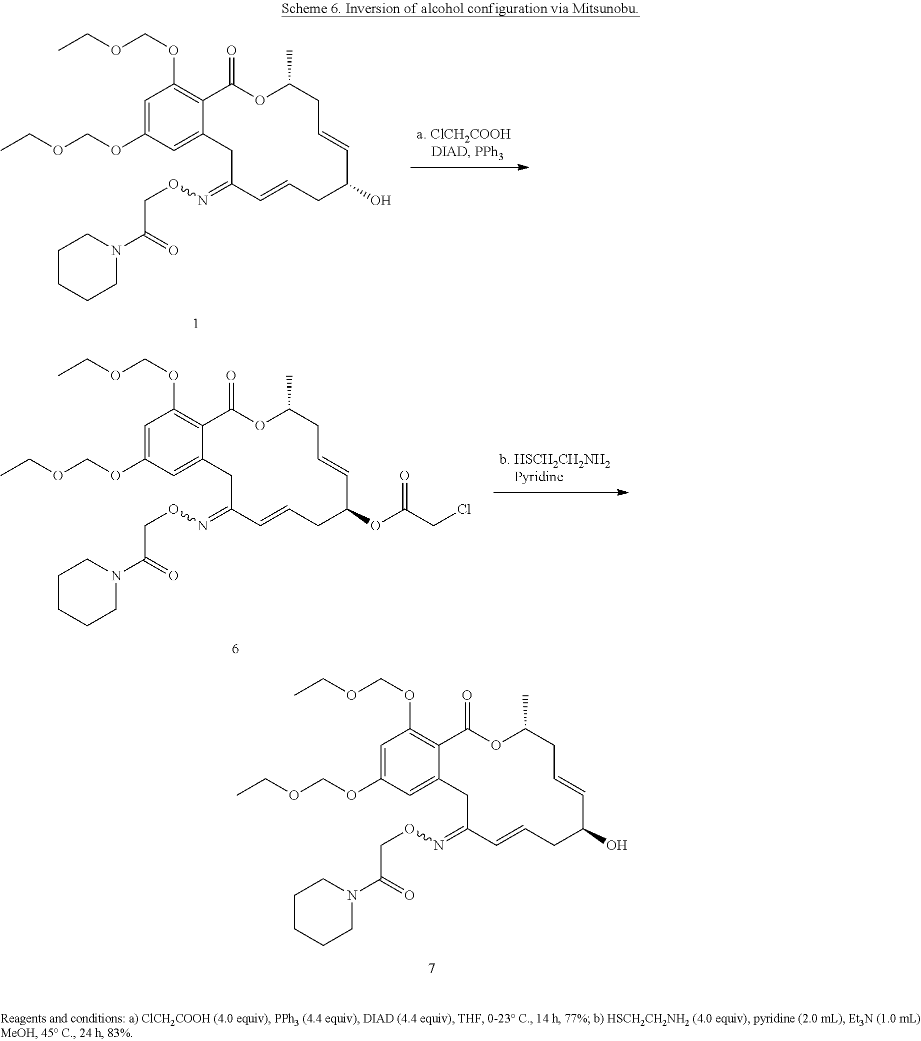 [SMILES: CCOCOc1cc2c(c(OCOCC)c1)C(=O)O[C@H](C)C/C=C/[C@H](O)C/C=C/C(=NOCC(=O)N1CCCCC1)C2 ; CCOCOc1cc2c(c(OCOCC)c1)C(=O)O[C@H](C)C/C=C/[C@@H](OC(=O)CCl)C/C=C/C(=NOCC(=O)N1CCCCC1)C2 ; CCOCOc1cc2c(c(OCOCC)c1)C(=O)O[C@H](C)C/C=C/[C@@H](O)C/C=C/C(=NOCC(=O)N1CCCCC1)C2]

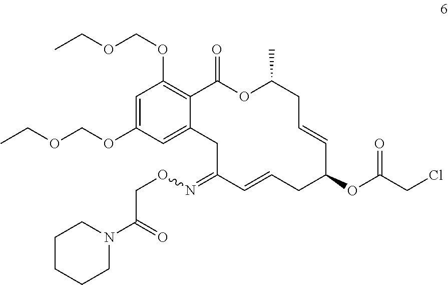 [SMILES: CCOCOc1cc2c(c(OCOCC)c1)C(=O)O[C@H](C)C/C=C/[C@@H](OC(=O)CCl)C/C=C/C(=NOCC(=O)N1CCCCC1)C2]

### Synthesis of Compound 6:

To a stirred ice-cooled solution of alcohol 1 (1.0 equiv, 200 mg, 0.34 mmol), chloroacetic acid (4.0 equiv, 127 mg, 1.36 mmol) and triphenylphosphine (4.4 equiv, 391 mg, 1.49 mmol) in THF (5 mL), DIAD (4.4 equiv, 0.294 mL, 1.49 mmol) was added dropwise and stirred at 23° C. for 14 hours. The reaction mixture was evaporated in vacuo and purified by flash chromatography (SiO₂, 3:7 to 1:1 EtOAc/Hexane), to give ester 6 (173 mg, 77%). R_{f}=0.32 (1:1 EtOAc/Hexane). HRMS (MALDI-TOF) m/z [M+H]⁺ calcd for C₃₃H₄₆ClN₂O₁₀: 665.2763. found: 665.2719.

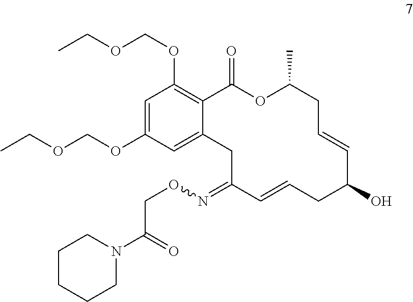 [SMILES: CCOCOc1cc2c(c(OCOCC)c1)C(=O)O[C@H](C)C/C=C/[C@@H](O)C/C=C/C(=NOCC(=O)N1CCCCC1)C2]

### Synthesis of Alcohol 7:

To a solution of ester 6 (1.0 equiv, 163 mg, 0.245 mmol) in methanol (3.0 mL), were added pyridine (2.0 mL), triethylamine (1.0 mL) and cystamine (4.0 equiv, 77 mg, 0.98 mmol) and the resulting mixture was stirred at 45° C. for 24 hours. Then the solvents were evaporated in vacuo and the crude purified by flash chromatography (SiO₂, 1:1 to 7:3 EtOAc)/Hexane), to afford alcohol 7 (120 mg, 83%). R_{f}=0.11 (8:2 EtOAc/Hexane); ¹H NMR (CDCl₃, 400 MHz) δ=6.88 (d, J=16.4 Hz, 1H), 6.74-6.72 (m, 2H), 6.66 (d, J=2.4 Hz, 1H), 6.59 (d, J=2.0 Hz, 1H), 6.18 (d, J=16.4 Hz, 1H), 6.07-5.91 (m, 2H), 5.64-5.53 (m, 4H), 5.21 (s, 8H), 5.20-5.16 (m, 2H), 4.83 (s, 4H), 4.46-4.42. (m, 3H), 3.78-3.69 (m, 10H), 3.60-3.59 (m, 6H), 3.46-3.42 (m, 6H), 3.28 (d, J=15.2 Hz, 1H), 2.46-2.37 (m, 8H), 1.68-1.58 (m, 12H), 1.46 (d, J=6 Hz, 6H), 1.30-1.20 (m, 12H); HRMS (MALDI-TOF) m/z [M+H]⁺ calcd for C₃₁H₄₅N₂O₉: 589.3047. found: 589.3033.

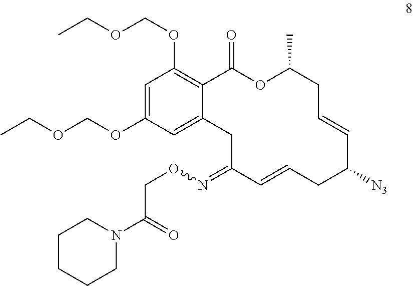 [SMILES: CCOCOc1cc2c(c(OCOCC)c1)C(=O)O[C@H](C)C/C=C/[C@H](N=[N+]=[N-])C/C=C/C(=NOCC(=O)N1CCCCC1)C2]

### Synthesis of Azide 8.

azide 8 was obtained following the same procedure as for azide 2, R_{f}=0.3 (1:1 EtOAc/Hexane); ¹H NMR (CDCl₃, 400 MHz) δ=6.87 (d, J=16.4 Hz, 1H), 6.74-6.72 (m, 2H), 6.65 (s, 1H), 6.59 (s, 1H), 6.14 (d, J=16.0 Hz, 1H), 5.78-5.60 (m, 4H), 5.40-5.34 (m, 2H), 5.24 (s, 8H), 5.20-5.14 (m, 2H), 4.83 (s, 4H), 4.53 (d, J=14.8 Hz, 1H), 3.71-3.68 (m, 10H), 3.61-3.57 (m, 5H), 3.48-3.39 (m, 5H), 3.11 (d, J=14.8 Hz, 1H), 2.52-2.48 (m, 2H), 2.42-2.35 (m, 5H), 1.68-1.58 (m, 12H), 1.46 (d, J=6.4 Hz, 6H), 1.30-1.20 (m, 12H) ppm; HRMS (MALDI-TOF) m/z [M+H]⁺ calcd for C₃₁H₄₄N₅O₈: 614.3112. found: 614.3126.

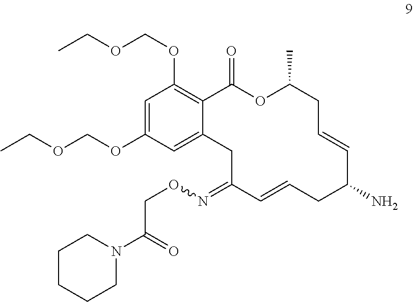 [SMILES: CCOCOc1cc2c(c(OCOCC)c1)C(=O)O[C@H](C)C/C=C/[C@H](N)C/C=C/C(=NOCC(=O)N1CCCCC1)C2]

### Synthesis of Amine 9.

Amine 9 was obtained following the same procedure as for amine 3. HRMS (MALDI-TOF) m/z [M+H]⁺ calcd for C₃₁H₄₆N₃O₈: 588.3207. found: 588.3225.

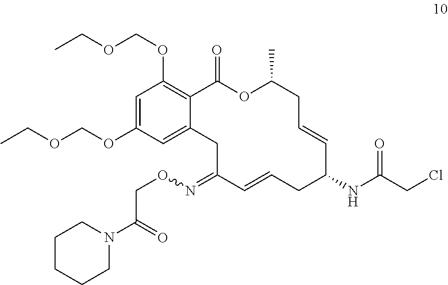 [SMILES: CCOCOc1cc2c(c(OCOCC)c1)C(=O)O[C@H](C)C/C=C/[C@H](NC(=O)CCl)C/C=C/C(=NOCC(=O)N1CCCCC1)C2]

### Preparation of Chloride 10.

Amine 9 was transformed into chloride 10 following the same procedure as descried before for chloride 6. HRMS (MALDI-TOF) m/z [M+H]⁺ calcd for C₃₃H₄₆ClN₂O₁₀: 664.2763. found: 664.2760.

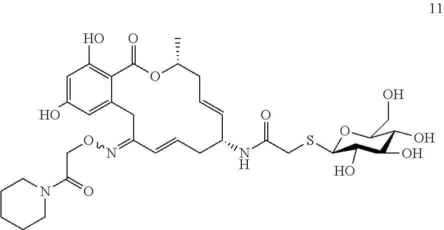

### Synthesis of Thio Glycoside 11.

Glycopochoxime derivative 11 was prepared following the same procedure as for the synthesis of compound 5. The crude product was purified by HPLC HPLC [Agilent Zorbax Eclipse XDB-C18 9.4 mm×25 mm column; flow rate 3.0 mL/min; linear gradient from 80% to 40% H₂O (0.01% TFA) in acetonitrile (0.01% TFA)] to give thio glycosides 11 (Z-isomer: 4.7 mg; E-isomer: 5.9 mg).

Glycopochoxime 11 Z-isomer ¹H NMR (CD₃OD, 400 MHz) δ=6.69 (d, J=16 Hz, 1H), 6.16 (d, J=2 Hz, 1H), 6.07 (d, J=2 Hz, 1H), 5.95-5.87 (m, 1H), 5.54-5.46 (m, 1H), 5.29 (dd, J=15.2, 8.8 Hz, 1H), 5.17-5.13 (m, 1H), 4.70 (d, J=4 Hz, 2H), 4.28 (d, J=10 Hz, 1H), 4.13-4.07 (m, 1H), 3.75 (d, J=12 Hz, 1H), 3.54-3.45 (m, 5H), 3.38 (t, J=5.2 Hz, 2H), 3.18-3.17 (m, 4H), 3.14-3.08 (m, 2H), 2.43-2.22 (m, 3H), 2.13-2.05 (m, 1H), 1.59-1.48 (m, 6H), 1.30 (d, J=6 Hz, 3H) ppm; ¹³C NMR (CD₃OD, 100 MHz) δ 170.3, 169.2, 168.3, 160.3, 158.8, 155.2, 139.3, 137.7, 131.9, 129.3, 120.0, 110.9, 106.8, 100.8, 85.1, 80.8, 78.1, 73.0, 70.9, 69.7, 61.5, 52.6, 48.5, 45.8, 42.8, 39.0, 38.7, 37.4, 35.5, 34.5, 32.5, 26.0, 23.9 ppm. HRMS (MALDI-TOF) m/z [M+H]⁺ calcd for C₃₃H₄₆N₃O₁₂S: 708.2724. found: 708.2747.

Glycopochoxime 11 E-isomer ¹H NMR (CD₃OD, 400 MHz) δ=6.21 (s, 1H), 6.19 (d, J=1.6 Hz, 1H), 6.05 (d, J=16 Hz, 1H), 5.97-5.89 (m, 1H), 5.67-5.60 (m, 1H), 5.41 (dd, J=15.2, 8.4 Hz, 1H), 5.29-5.23 (m, 1H), 4.42 (s, 2H), 4.39 (d, J=5.6 Hz, 1H), 4.20-4.15 (m, 1H), 3.88 (d, J=11.6 Hz, 1H), 3.66-3.62 (m, 2H), 3.59-3.50 (m, 4H), 3.47-3.44 (m, 4H), 3.31 (s, 2H), 2.55-2.51 (m, 1H), 2.44-2.36 (m, 2H), 2.22-2.15 (m, 1H), 1.72-1.62 (m, 6H), 1.45 (d, J=6 Hz, 3H) ppm; ¹³C NMR (CD₃OD, 100 MHz) δ 170.2, 169.1, 168.0, 160.2, 157.9, 137.8, 134.0, 131.8, 129.5, 126.8, 106.8, 100.6, 85.0, 80.8, 78.0, 72.9 71.5, 71.0, 70.0, 61.5, 52.8, 48.5, 45.9, 42.8, 39.1, 36.9, 32.5, 28.2, 26.1, 25.3, 24.0, 19.0 ppm; HRMS (MALDI-TOF) m/z [M+H]⁺ calcd for C₃₃H₄₆N₃O₁₂S: 708.2724. found: 708.2790.

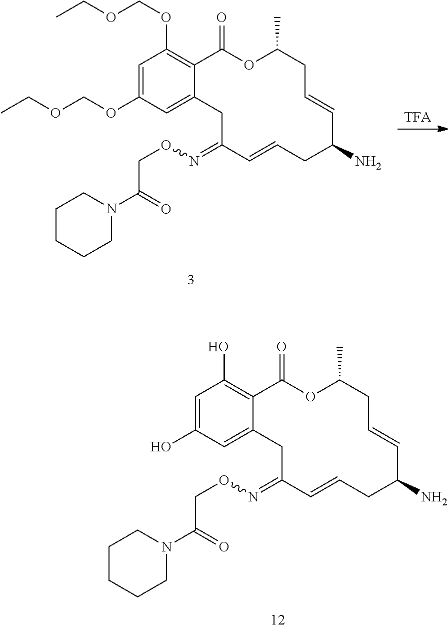 [SMILES: CCOCOc1cc2c(c(OCOCC)c1)C(=O)O[C@H](C)C/C=C/[C@@H](N)C/C=C/C(=NOCC(=O)N1CCCCC1)C2 ; C[C@@H]1C/C=C/[C@@H](N)C/C=C/C(=NOCC(=O)N2CCCCC2)Cc2cc(O)cc(O)c2C(=O)O1 ; "TFA"]

### Synthesis of Amine 12.

Amine 3 (15 mg) was dissolved in TFA/m-cresol 1:1 (200 μL/200 μL) and stirred at RT for 5 min. The reaction mixture was diluted with 10 mL H₂O, lyophilized and purified by HPLC [Agilent Zorbax Eclipse XDB-C18 9.4 mm×25 mm column; flow rate 2.0 mL/min; linear gradient from 80% to 60% H₂O (0.01% TFA) in acetonitrile (0.01% TFA)] to obtain 0.9 mg of pure amine 12 Z isomer (Rt=12.2 min) and 1.8 mg of amine 12 E isomer (Rt=13.4 min)

Amine 12 Z-isomer ¹H NMR (CDCl₃, 400 MHz) δ=6.67 (d, J=16.4 Hz, 1H), 6.53 (d, J=2.0 Hz, 1H), 6.29 (d, J=2.0 Hz, 1H), 6.08-5.95 (m, 1H), 5.54-5.47 (m, 2H), 5.40-5.35 (m, 1H), 4.62-4.59 (m, 2H), 4.34 (d, J=15.6 Hz, 1H), 3.82 (d, J=15.2 Hz, 1H), 3.55-3.54 (m, 2H), 3.37 (s, 2H), 2.71-2.66 (m, 2H), 2.49-2.45 (m, 2H), 2.33-2.25 (m, 2H), 1.93 (s, 3H), 1.66-1.58 (m, 6H), 1.43 (d, J=6.4 Hz, 3H) ppm. HRMS (MALDI-TOF) m/z [M+H]⁺ calcd for C₂₅H₃₄N₃O₆:472.2369. found: 472.2332.

Amine 12 E-isomer ¹H NMR (CDCl₃, 400 MHz) δ=6.45 (s, 1H), 6.30 (d, J=1.6 Hz, 1H), 5.95 (s, 1H), 5.55-5.50 (m, 3H), 5.41-5.35 (m, 1H), 4.91-4.79 (m, 2H), 4.62-4.56 (m, 1H), 4.37 (d, J=4.4 Hz, 1H), 3.56 (s, 2H), 3.37 (s, 2H), 2.73-2.65 (m, 2H), 2.46-2.21 (m, 4H), 1.99 (s, 3H), 1.66-1.59 (m, 6H), 1.44 (d, J=6.4 Hz, 3H) ppm; HRMS (MALDI-TOF) m/z [M+H]⁺ calcd for C₂₅H₃₄N₃O₆:472.2369. found: 472.2353.

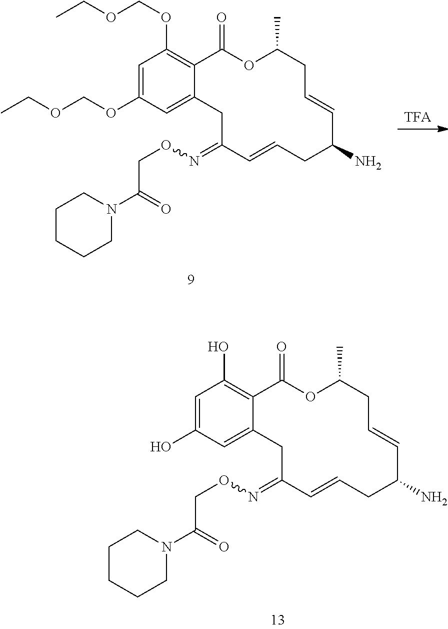 [SMILES: CCOCOc1cc2c(c(OCOCC)c1)C(=O)O[C@H](C)C/C=C/[C@@H](N)C/C=C/C(=NOCC(=O)N1CCCCC1)C2 ; C[C@@H]1C/C=C/[C@H](N)C/C=C/C(=NOCC(=O)N2CCCCC2)Cc2cc(O)cc(O)c2C(=O)O1 ; "TFA"]

Synthesis of amine 13.

Amine 9 (10 mg) was dissolved in TFA/m-cresol 2:1 (100 μL/50 μL) and stirred at RT for 5 min. The reaction mixture was diluted with 10 mL H₂O, lyophilized and purified by HPLC in an attempt to separate both isomers [Agilent Zorbax Eclipse XDB-C18 9.4 mm×25 mm column; flow rate 2.0 mL/min; linear gradient from 80% to 60% H₂O (0.01% TFA) in acetonitrile (0.01% TFA)] without success, to give amine 13 as a mixture of isomers (1.7 mg); ¹H NMR (CD₃OD, 400 MHz) δ=6.91 (d, J=16 Hz, 1H), 6.27 (d, J=1.6 Hz, 1H), 6.19 (s, 3H), 6.12 (d, J=15.6 Hz, 1H), 5.91-5.78 (m, 4H), 5.46-5.40 (m, 2H), 5.29-5.23 (m, 2H), 4.42 (d, J=15.2 Hz, 1H), 3.68-3.49 (m, 13H), 2.69-2.47 (m, 8H), 2.34-2.28 (m, 4H), 1.72-1.71 (m, 6H), 1.63-1.62 (m, 6H), 1.47 (d, J=6 Hz, 6H) ppm; HRMS (MALDI-TOF) m/z [M+H]⁺ calcd for C₂₅H₃₄N₃O₆:472.2369. found: 472.2324.

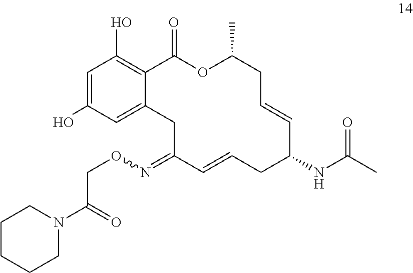 [SMILES: CC(=O)N[C@H]1/C=C/C[C@@H](C)OC(=O)c2c(O)cc(O)cc2CC(=NOCC(=O)N2CCCCC2)/C=C/C1]

### Synthesis of Acetamide 14.

Amine 9 was dissolved in dichloromethane (1 mL) and cooled to 0° C., i-Pr₂EtN (5.0 equiv, 1.9 μL, 0.085 mmol) and acetic anhydride (5.0 equiv, 8.6 μL, 0.08 mmol) were added sequentially and stirred for 10 min. The reaction mixture was quenched with aqueous NaHCO₃ and washed with brine, dried and evaporated in vacuo. The crude acetyl derivative was dissolved in methanol (3 mL) and PS—SO₃H (10.0 equiv, 56 mg, 0.17 mmol, 3.0 mmol/g) was added and stirred at 23° C. for 16 hours. The reaction mixture was filtered, and evaporated in vacuo. The crude product was purified by HPLC [Agilent Zorbax Eclipse XDB-C18 9.4 mm×25 mm column; flow rate 2.0 mL/min; linear gradient from 70% to 50% H₂O (0.01% TFA) in acetonitrile (0.01% TFA)] to give acetamide 14 (Z-isomer: Rt=13.8 min, 5.4 mg; E-isomer: Rt=14.7 min, 5.8 mg).

Acetamide 14 Z-isomer: ¹H NMR (CDCl₃, 400 MHz) δ=6.67 (d, J=16.4 Hz, 1H), 6.53 (d, J=2.0 Hz, 1H), 6.29 (d, J=2.0 Hz, 1H), 6.08-5.95 (m, 1H), 5.54-5.47 (m, 2H), 5.40-5.35 (m, 1H), 4.62-4.59 (m, 2H), 4.34 (d, J=15.6 Hz, 1H), 3.82 (d, J=15.2 Hz, 1H), 3.55-3.54 (m, 2H), 3.37 (s, 2H), 2.71-2.66 (m, 2H), 2.49-2.45 (m, 2H), 2.33-2.25 (m, 2H), 1.93 (s, 3H), 1.66-1.58 (m, 6H), 1.43 (d, J=6.4 Hz, 3H) ppm. HRMS (MALDI-TOF) m/z [M+H]⁺ calcd for C₂₇H₃₆N₃O₇: 514.2475. found: 514.2400.

Acetamide 14 E-isomer: ¹H NMR (CDCl₃, 400 MHz) δ=6.45 (s, 1H), 6.30 (d, J=1.6 Hz, 1H), 5.95 (s, 1H), 5.55-5.50 (m, 3H), 5.41-5.35 (m, 1H), 4.91-4.79 (m, 2H), 4.62-4.56 (m, 1H), 4.37 (d, J=4.4 Hz, 1H), 3.56 (s, 2H), 3.37 (s, 2H), 2.73-2.65 (m, 2H), 2.46-2.21 (m, 4H), 1.99 (s, 3H), 1.66-1.59 (m, 6H), 1.44 (d, J=6.4 Hz, 3H) ppm; HRMS (MALDI-TOF) m/z [M+H]⁺ calcd for C₂₇H₃₆N₃O₇: 514.2475. found: 514.2464.

### Asymmetric Synthesis of Pochonin Oxime Analogs:

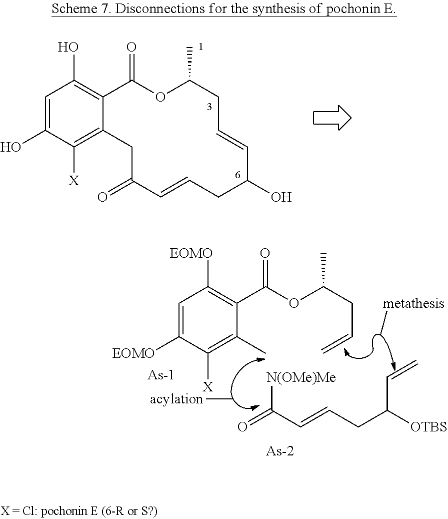

The asymmetric synthesis of pochonin E and F was envisioned to proceed as shown in Scheme 7 and was based on the logic of previous pochonin synthesis thus requiring known fragment As-1 and chiral fragment As-2 with either S or R stereochemistry. Fragment As-2 could be expediently accessed using the methodology developed by the Denmark group for the activation of mild Lewis acids with chiral Lewis bases. Salient features of this reaction are the extremely low catalyst loading (1 mol %) and excellent regioselectivity coupled to high enantiomeric excess obtained with vinylogous nucleophiles. From a practical standpoint, both enantiomers of the catalyst are commercially available. However, the required 2-buteneal substrate had not been previously reported with vinylogous nucleophiles and aliphatic acrylic aldehydes tend to be poor substrates. Reaction of vinylogous slilylenol ether As-3 with 2-buteneal and SiCl₄ catalyzed by Denmark's catalyst afforded the desired compound As-4 as the only regioisomer in 45% yield (Scheme 8). The lower yield of the reaction for 2-buteneal had been previously noted with silyl enol ether. The alcohol group was then protected as TBS ether and the ester converted to the Weinreb amide under the action of the magnesium chloride salt of Weinreb amine in excellent yield. The R-As-2 stereoisomer was prepared starting with the (R,R)-As-5 catalyst and its enantiomer S-As-2 using the (S,S)-As-5 catalyst.

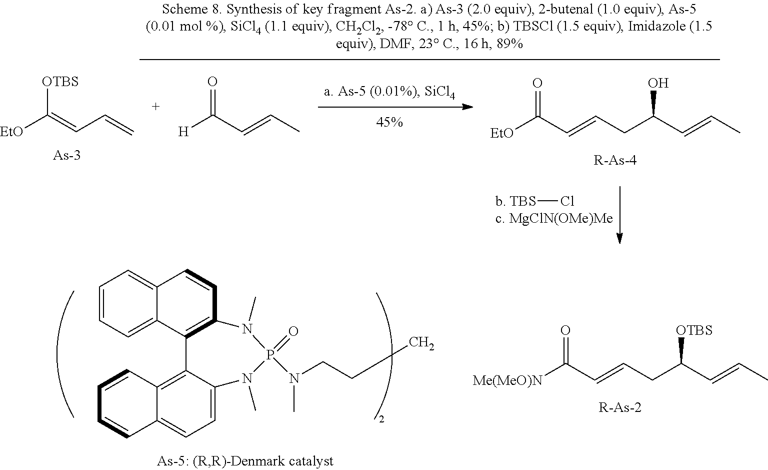

As shown in Scheme 9, intermediates As-1a and As-1b were individually treated with LDA to deprotonated the benzylic position and engaged in a reaction with either R-As-2 or S-As-2 to obtain the product As-6a and As-6b respectively in acceptable yield and consistent with previous use of this transformation. ENREF_20 Notably, δ-elimination of the hydroxyl ether from the α,β conjugated system was not observed under the basic conditions of the reaction despite excess of LDA. Cyclization under the action of second generation Grubbs catalyst (Grubbs II) for each permutation of isomer and aryl substituent of As-6 followed by a sulfonic acid catalyzed deprotection of the EOM with concomitant deprotection of the silyl ether afforded the two different distereosiomers of pochonin E (As-7b) and pochonin F (As-7a) respectively. Surprisingly, the yield of the metathesis (48%-65%) was lower than in previous cases lacking the allylic substitution. It has been shown that conversion of pochonin D ketone to an oxime (pochoxime), particularly with 2-(aminooxy) piperidyl acetamide afford significant improvement in affinity to Hsp90 and cellular efficacy in promoting the depletion of Hsp90 client. For the preparation of the pochoxime analogs of pochonin E and F as well as their epimers, intermediates As-6a and As-6b were condensed with 2-(aminooxy) piperidyl acetamide As-8 in pyridine to afford both epimers of products As-9a and As-9b respectively as a mixture of oxime geometries. Contrarily to the ketones As-6a and As-6b, oximes As-9 afforded excellent yield in the metathesis (75-95%). Global deprotection under the action of sulfonic acid resin afforded a separable mixture of E and Z oxime isomers of pochoxime E and F as well as their epimers (As-10b and As-10a respectively).

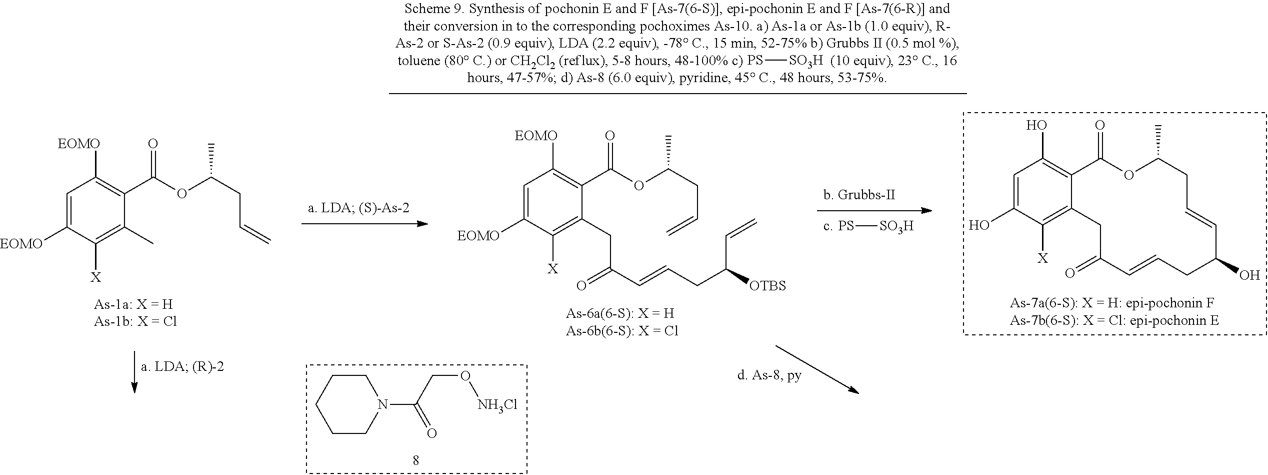

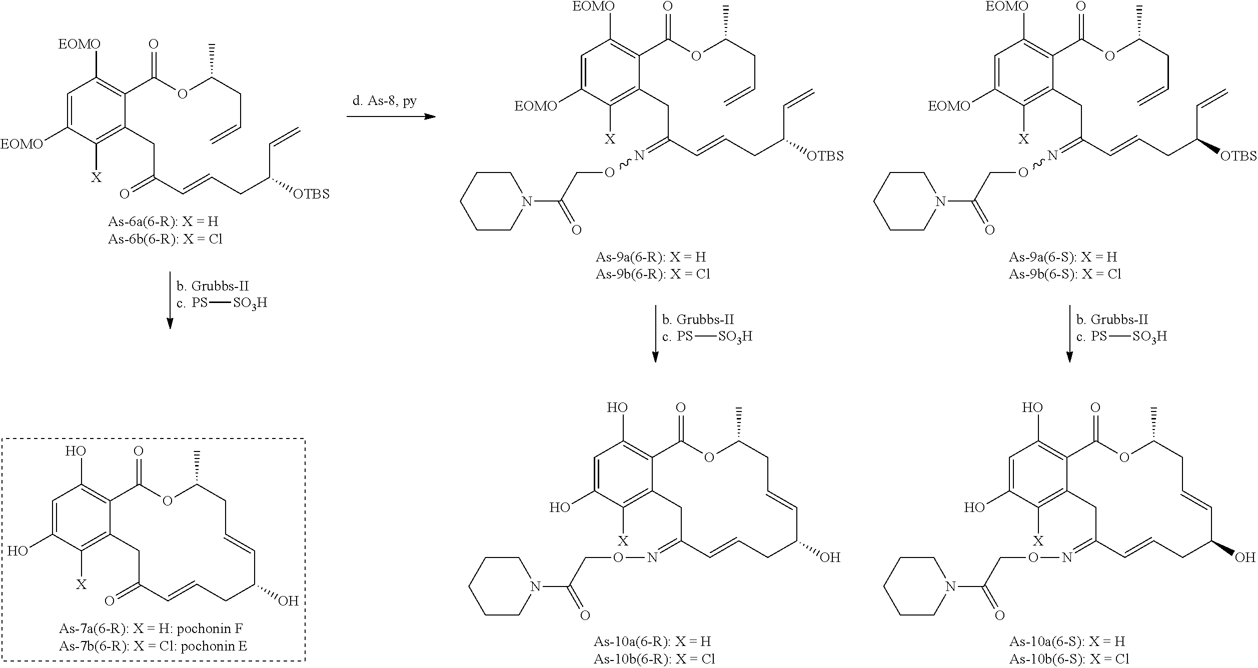

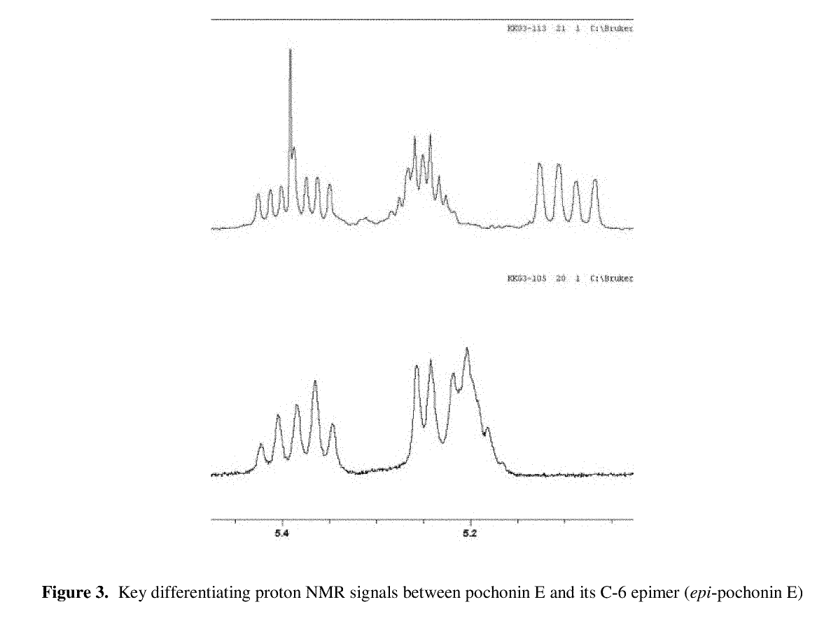

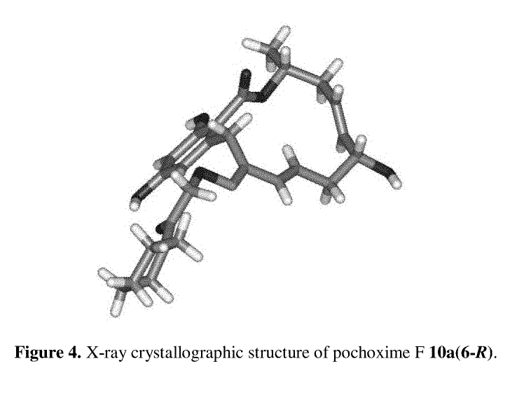

Having access to pochonins E and F with both R and S stereochemistry at the C-6 hydroxyl, the NMR of the synthetic compounds were compared to the natural products (Table 2). In deuterated methanol, the most salient difference in the NMR of pochoxime E was the chemical shift of the proton of carbon 1. For the S-isomer, this proton was between the two alkenic protons of C-4 and C-5 while for the R isomer, it was slightly upfiled of the alkenic protons (FIG. 3). Also notable are differences in chemical shifts between the both protons of C-11. In the case of the S-isomer, they are sufficiently resolved to give the expected pair of doublets. In the case of the R-isomer, they are close and only give a hint of their splitting showing an apparent singlet. In the case of pochonin F which was reported in deuterated acetone, the distinction between the C-1 proton is less pronounced but a similar difference between the C-11 protons are visible with the S-isomer having a larger difference in chemical shift between both protons. The NMR comparison between both synthetic epimers at C-6 and the natural product leads to the conclusion that the natural compound has the R stereochemistry at C-6. Crystallization of the pochoxime isomer As-10a (E-oxime isomer) emanating from the intermediate which yielded the pochonin F epimer afforded diffracting crystals (FIG. 4) and clearly showed the R stereochemistry at C-6.

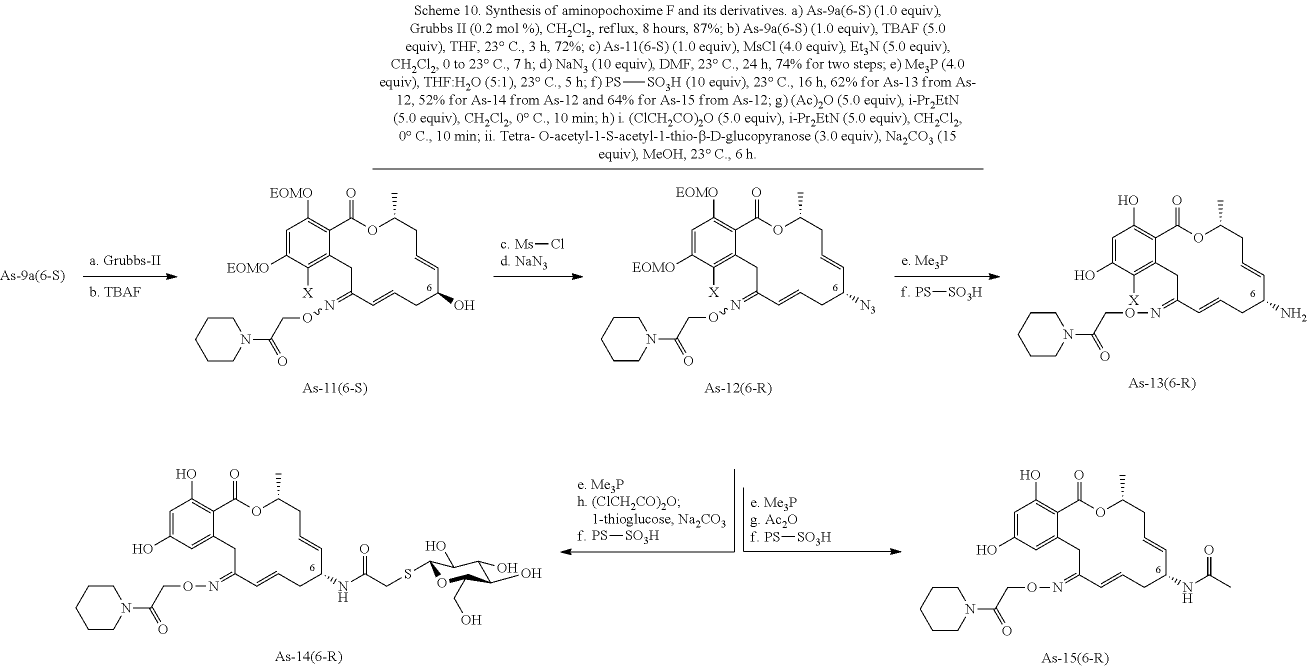

In efforts to further explore modifications at the C-6 position of the pochoxime scaffold, the fully protected pochoxime obtained from the metathesis cyclization of As-9a was treated with TBAF to selectively remove the silyl group thus affording As-11. The hydroxyl group was converted to an azide in two steps (Ms-Cl; NaN₃) to obtain As-12 which was reduced to an amino group with Trimethylphosphine. Global deprotection of EOM groups with sulfonic acid resin thus afforded C-6 amino pochoxime As-13. Alternative strategies to convert the hydroxyl using palladium catalyzed n-ally chemistry via its acetylated or carbonated form where not productive. The key azide As-12, following a reduction to the amine, could also be derivatized as a chloroacetamide and conjugate to 1-β-thioglucose to afford As-14. The same sequence using a simple acetylation afforded As-15. While the chemistry in Scheme 10 is shown starting with the S isomer As-9a, the same procedures were carried out with the R isomer thus affording products As-13 to As-15 as the S isomers at C-6. All the products were obtained as a mixtures of oxime geometries which were separated by chromatography.

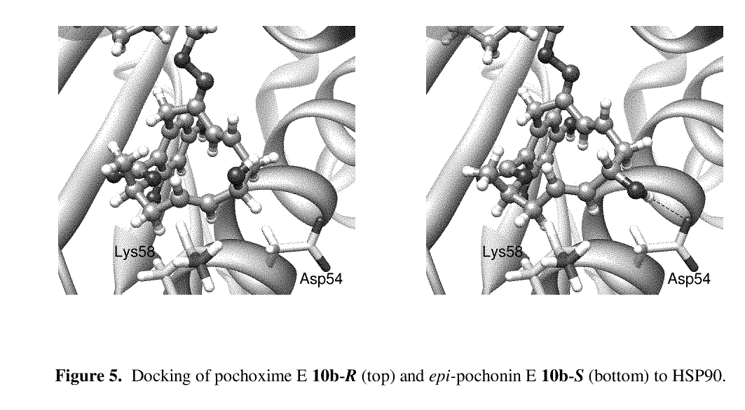

We next measured the affinity of the pochoximes for human Hsp90a using a previously reported competition assay. In parallel, the impact on the conformational profile of modifications at C-6 was evaluated for compound As-10 (6-S and 6-R diastereoisomers with and without chlorine) and As-13 (6-S and 6-R diastereoisomers). Each molecule was simulated by molecular dynamics with the Merck molecular Force Field in the CHARMM program to analyze its conformation profile. As previously described this analysis led to the identification of three main conformations: an L-shape conformation, which is the bioactive conformation of radicicol and pochoxime derivatives (see FIG. 5), an essentially planar (P-shape) conformation, and an L′-shape conformation that mainly differs from the L-shape one by the fact that the macrocycle is positioned on the opposite side of the aromatic cycle. The L′-shape conformer was found to be the energetically most favorable for all the studied molecules and was indeed observed in the crystal structure of compound As-10a-6R (FIG. 4). Depending on the molecule, the L-shape conformation was energetically disfavored by 0.8 to 2.7 kcal mol⁻¹ (Table 2). In addition, each compound was docked in Hsp90 using Autodock 4, AutodockVina and the “Attracting cavities” algorithm of EADock, based on the Hsp90 conformation observed with co-crystals of Hsp90 with pochoxime A and B (pdb ID: 2INW and 3INX respectively). All programs were found to reproduce the experimental binding mode determined by X-ray crystallography for three test compounds (radicicol, and pochoxime A and B), illustrating the efficiency of these approaches. The “attracting cavities” approach of EADock predicted a binding mode similar to that of the 3INW ligand for the new pochoxime derivatives, thus adopting an L-shape conformer, whatever the starting conformation of the input molecule that was given to seed the docking process. These poses were confirmed by the Autodock 4 and AutodockVinaruns that were started using the L-shape conformers. Due to a sub-optimal exploration of the conformational space of the pochoxime macrocycles, the Autodock 4 and AutodockVina runs started from the P-shape conformers led to unbound poses exhibiting more unfavorable scores (data not shown). The binding affinities of the new pochoxime derivatives, in their calculated binding modes, were estimated using the approach previously described. Results are in good agreement with the experimentally determined affinities. As seen in table 2, it is clear that substitution at the C-6 position has a detrimental impact in terms of the energetic penalty incurred for adopting the bioactive conformation. In this respect, it is interesting to note that the conformation of the macrocycle obtained from the crystallographic structure of As-10a-6R is not the bioactive conformation. The most pronounced energetic penalty observed is for the amino group at the C-6 in the R-stereochemistry. The fact that the amino group is protonated a physiological pH make this substituent more sterically demanding than the corresponding hydroxyl and results in the largest calculated energetic penalty (2.68 kcal/mol) within this series. Correspondingly, this compound has the worst affinity for Hsp90 (229 nM compared to 38 nM for the corresponding hydroxyl substitute analog). Substitution of the C-6 amino group with the glycan (As-14) is tolerated, preferably with the S stereochemistry (32 nM vs 90 nM for the R isomer) which minimized the energetic penalty of adopting the bioactive conformation. Docking of pochoxime F and its C-6 epimer in Hsp90 shows that, in the R stereochemistry (pochoxime F), the hydroxyl group is pointing towards the solvent however, in the S-stereochemistry (epi-pochoxime F), it is within hydrogen-bond distance from the aspartic acid residue (FIG. 5). This interaction could rationalize the fact that this isomer of pochoxime F is the most potent in the series (14 nM). Similarly, it is interesting to note that the epi-aminopochoxime E has significantly better affinity to Hsp90 than aminopochoxime E and should also benefit from this additional interaction with Hsp90's Asp54.

TABLE 2
Comparison of ¹H NMR data for the different C-6 configurations corresponding to pochonin E and F.

| | 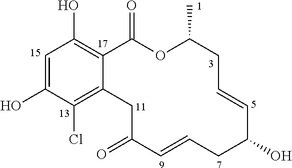 | | As-7b(6R) | | As-7b(6S) | |
|---|---|---|---|---|---|---|
| | natural pochonin E | | synthetic pochonin E | | synthetic epi-pochonin E | |
| car- | δ_{H}, mlti (J in Hz) | | δ_{H}, mlti (J in Hz) | | δ_{H}, mlti (J in Hz) | |
| bon | MeOD-d4 | | MeOD-d4 | | MeOD-d4 | |
| 1 | 1.19 | d (6.4) | 1.19 | d (6.4) | 1.19 | d (6.4) |
| 2 | 5.20 | m | 5.20 | m | 5.25 | m |
| 3 | 2.47 | ddd (14.3, 7.6, 3.7) | 2.47 | ddd (14.3, 7.5, 3.6) | 2.47 | ddd (12.2, 9.8, 3.8) |
| | 2.16 | ddd (14.3, 7.6, 5.5) | 2.16 | ddd (14.3, 7.5, 5.6) | 2.21 | ddd (12.2, 5.2, 1.4) |
| 4 | 5.38 | dt (15.3, 7.6) | 5.38 | dt (15.2, 7.5) | 5.38 | ddd (15.1, 9.8, 5.2) |
| 5 | 5.23 | dd (15.3, 6.1) | 5.24 | dd (15.2, 6.0) | 5.09 | dd (15.1, 7.9) |
| 6 | 4.24 | m | 4.23 | m | 4.09 | m |
| 7 | 2.36 | ddd (12.8, 7.3, 3.4) | 2.36 | ddd (12.8, 7.3, 3.6) | 2.44 | m |
| | 2.25 | dt (12.8, 7.3) | 2.25 | dt (12.8, 7.3) | 2.00 | dt (11.7, 10.1) |
| 8 | 6.69 | dt (15.9, 7.3) | 6.69 | dt (15.9, 7.3) | 6.61 | ddd (15.8, 10.1, 6.04) |
| 9 | 5.80 | d (15.9) | 5.80 | d (15.9) | 5.74 | d (15.8) |
| 11 | 4.14 | d (17.7) | 4.17 | d (17.6) | 4.08 | d (17.8) |
| | 4.10 | d (17.7) | 4.09 | d (17.6) | 3.91 | d (17.8) |
| 15 | 6.35 | s | 6.37 | s | 6.39 | s |
| | 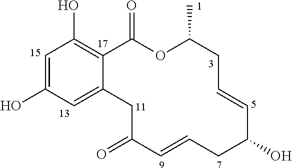 | | As-7A(6R) | | As-7a(6S) | |
| | natural pochonin F[11] | | synthetic pochonin F | | synthetic epi-pochonin F | |
| car- | δ_{H}, mlti (J in Hz) | | δ_{H}, mlti (J in Hz) | | δ_{H}, mlti (J in Hz) | |
| bon | Acetone-d6 | | Acetone-d6 | | Acetone-d6 | |
| 1 | 1.28 | d (6.7) | 1.28 | d (6.5) | 1.30 | d (6.7) |
| 2 | 5.38 | m | 5.38 | m | 5.31 | m |
| 3 | 2.65 | ddd (14.0, 7.9, 4.3) | 2.65 | ddd (14.1, 7.8, 4.3) | 2.68 | ddd (14.1, 9.6, 3.9) |
| | 2.24 | ddd (14.0, 7.9, 4.3) | 2.24 | ddd (14.1, 7.8, 4.3) | 2.29 | ddd (14.1, 5.2, 3.7) |
| 4 | 5.52 | dt (15.3, 7.9) | 5.52 | dt (15.3, 8.1) | 5.49 | ddd (15.2, 9.6, 5.2) |
| 5 | 5.37 | m | 5.37 | m | 5.27 | m |
| 6 | 4.41 | m | 4.40 | m | 4.25 | m |
| 7 | 2.48 | ddd (14.0, 7.3, 4.9) | 2.48 | ddd (12.8, 7.3, 4.0) | 2.50 | m |
| | 2.34 | dt (14.0, 8.5) | 2.34 | dt (12.8, 8.4) | 2.13 | dt (12.2, 9.2) |
| 8 | 6.73 | ddd (15.9, 8.5, 7.3) | 6.73 | ddd (15.9, 8.5, 7.3) | 6.66 | ddd (15.7, 9.2, 6.6) |
| 9 | 5.89 | d (15.9) | 5.88 | d (15.9) | 5.85 | d (15.7) |
| 11 | 4.04 | d (16.5) | 4.04 | d (16.8) | 4.13 | d (16.9) |
| | 3.98 | d (16.5) | 3.98 | d (16.8) | 3.70 | d (16.9) |
| 15 | 6.31 | d (2.4) | 6.30 | d (2.4) | 6.31 | d (2.4) |

TABLE 3
Calculated free energetic penalty to adopt the bioactive conformation, predicted K_{D}
and measure K_{D}.

| 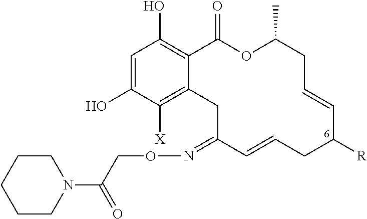 | Energetic penalty for adopting the bioactive conformation (kcal/mol) | Calculated K_{D} SwissParam (nM) | Measure K_{D} against human HSP90α (nM) |
|---|---|---|---|
| radicicol | 0.0 | | 140 |
| pochoxime D: X = Cl; R = H | 0.36 | 35 | 22 |
| epi-pochoxime E (As-10b): X = Cl; R = S—OH | 0.98 | 42 | 38 |
| pochoxime E (As-10b): X = Cl; R = R—OH | 0.78 | 43 | 34 |
| epi-pochoxime F (As-10a): X = H; R = S—OH | 2.07 | 25 | 14 |
| pochoxime F (As-10a): X = H; R = R—OH | 1.45 | 32 | 22 |
| epi-aminopochoxime E (As-13): X = H; R = S—NH₂ | 1.96 | 33 | 34 |
| aminopochoxime E (As-13): X = H; R = R—NH₂ | 2.68 | 29 | 229 |
| As-14(6-R) | NC | NC | 90 |
| As-14(6-S) | NC | NC | 32 |
| As-15(6-R) | NC | NC | 343 |

The concise synthesis presented herein afforded rapid access to pochonin E and F as well as their epimers at the allylic hydroxyl group (C-6). Comparison between the reported proton NMR data of the natural products and the synthetic compounds clearly established the stereochemistry of the hydroxyl group to be the R-isomer. Furthermore, the described chemistry enabled further exploration into the structure activity relationship of this pharmacophore for Hsp90 inhibition, an important therapeutic target. Despite an unfavorable conformational bias imposed by modifications at C-6, epi-pochoxime F (As-10a) stands as the most potent Hsp90 ligand in the pochoxime series (14 nM), which may be rationalized by the formation of a productive hydrogen bond to an aspartic acid residue in Hsp90. Finally, the pochoxime-glucose conjugate As-14(6-S) was shown to be a potent Hsp90 ligand (32 nM) and may be useful to direct the inhibitor to metabolically demanding malignant cells by an active uptake mechanism.

### General Techniques.

All reactions were carried out under a nitrogen atmosphere with dry solvents under anhydrous conditions, unless otherwise noted. Anhydrous solvents were obtained by passing them through commercially available alumina columns (Innovative technology, Inc., MA). Reactions were monitored by LC-MS or thin layer chromatography (TLC) carried out on 0.25 mm E. Merck silica gel plates (60E-254) using UV light as visualizing agent and 10% ethanolic phosphomolybdic acid or vanillin solution and heat as developing agents. E. Merck silica gel (60, particle size 0.040-0.063 mm) was used for flash column chromatography. NMR spectra were recorded on Bruker Advance-400 instrument at 400 (¹H), 100 (¹³C) MHz. Chemical shifts are given in parts per million (ppm) and calibrated using residual undeuterated solvent as an internal reference. The following abbreviations were used to explain the multiplicities: s=singlet, d=doublet, dd=doublet of doublets, t=triplet, q=quartet, m=multiplet, b=broad. LC-MS were recorded using an Agilent 1100 HPLC. Unless otherwise stated, a Supelco C8 (5 cm×4.6 mm, 5 mm particles) column was used with a linear elution gradient from 95% H₂O (0.5% HCO₂H) to 100% MeCN in 8 minutes at a flow rate of 0.5 mL/min. (S,S)-Denmark catalyst: (S,S)—N,N′-Bis[4,5-dihydro-3,5-dimethyl-4-(3H-dinaphtho[2,1-d:1′,2′-f][1,3,2]-2-oxo-diazaphosphepino)]-N,N′-dimethyl-1,5-pentanediamine, DMF=N,N-dimethylformamide, Grubbs II=benzylidene[1, 3-bis (2, 4, 6-trimethylphenyl)-2-imidazolidinylidene] dichloro (tricyclohexylphosphine) ruthenium, PS—SO₃H=sulfonic acid polystyrene, TBAI=tetrabutylammonium iodide, TBS=t-Butyl dimethylsilyl, TBSCl: t-Butyl dimethylsilyl chloride, TFA=trifluoroacetic acid, THF=tetrahydrofuran.

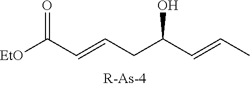

### Synthesis of the Enantiomerically Pure Aldol Adduct R-As-4.

A flame dried 25 ml two necked round bottomed flask, was charged with (R,R)-Denmark catalyst (8.4 mg, 0.01 mmol, 0.01%), crotonaldehyde (84 μl, 1.0 mmol, 1.0 equiv), and 5.0 mL of dry CH₂Cl₂ under nitrogen atmosphere. This solution was cooled down to −78° C. with a dry ice-acetone bath. After 5 minutes of stirring SiCl₄ (123 μl, 1.1 mmol, 1.1 equiv) was added dropwise, and the solution was stirred for additional 5 minutes. To this solution was added neat silylketene acetal As-3 (293 mg, 1.2 mmol, 1.2 equiv) in a dropwise manner over 5 minutes period. After 3 hours, Et₃N (459 μl, 3.3 mmol, 3.3 equiv) in 2.0 ml of dry methanol was added by cannula at −78° C., the mixture was stirred for 1 hour at room temperature, then quenched with saturated NH₄Cl_{aq} solution (10 mL), and kept stirring for an additional hour. The mixture was then extracted with CH₂Cl₂ (3×30 mL) and the combined organic layers were dried with Na₂SO₄. Filtration and evaporation of the solvents under reduced pressure followed by flash chromatography (silica gel, 80/20 petroleum ether/EtOAc) afforded the desired compound R-As-4 as a yellow oil in 45% yield (86 mg). Rf=0.18 (80/20 petroleum ether/EtOAc); ¹H (CDCl₃, 400 MHz, 23° C.) δ=6.99 (dt, J=15.0, 7.2 Hz, 1H); 6.46 (dd, J=15.0, 1.6 Hz, 1H); 5.76 (m, 1H); 5.56 (m, 1H); 4.25-4.11 (m, 3H); 2.46-2.43 (m, 2H); 1.73 (d, J=6.4 Hz, 3H); 1.32 (t, J=7.2 Hz, 3H) ppm; OH signal is missing. ¹³C NMR (CDCl₃, 100 MHz, 25° C.) δ=166.3, 144.7, 132.9, 127.7, 123.8, 71.51, 60.2, 40.1, 17.6, 14.2 ppm.

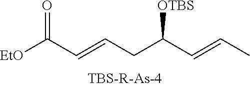 [SMILES: C/C=C/[C@@H](C/C=C/C(=O)OCC)O[Si](C)(C)C(C)(C)C]

### Synthesis of Protected Aldol Adduct TBS-R-As-4.

To the solution of the aldol adduct R-As-4 (163 mg, 0.814 mmol, 1.0 equiv) in dry DMF (5.0 mL), was added imidazole (72 mg, 1.053 mmol, 1.3 equiv) at room temperature. After 5 minutes of stirring TBSCl (147.2 mg, 0.976 mmol, 1.2 equiv) was added and stirred for 16 hours. After evaporation of the solvent, the residue was purified by flash chromatography (silica gel, 90/10 petroleum ether/EtOAc) to give the protected alcohol TBS-R-As-4 in 89% yield (216 mg). Rf=0.82 (80/20 petroleum ether/EtOAc); ¹H NMR (CDCl₃, 400 MHz, 23° C.) δ=6.99 (dt, J=15.0, 7.2 Hz, 1H); 6.46 (dd, J=15.0, 1H); 5.76 (m, 1H); 5.56 (m, 1H); 4.25-4.11 (m, 3H); 2.46-2.43 (m, 2H); 1.73 (d, J=6.4 Hz, 3H); 1.32 (t, J=7.2 Hz, 3H); 0.91 (s, 9H); 0.06 (d, J=7.6 Hz, 6H) ppm; ¹³C NMR (CDCl₃, 100 MHz, 25° C. δ=166.4, 145.7, 133.7, 125.8, 123.1, 72.5, 60.1, 41.4, 25.8 (×3), 25.6, 17.5, 14.2, −4.3, −4.8 ppm.

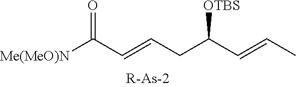

### Synthesis of Weinreb Amide R-As-2.

To a solution of the protected alcohol TBS-R-As-4 (215 mg, 0.688 mmol, 1.0 equiv) in dry THF (8.0 mL), N,O-dimethylhydroxylamine hydrochloride salt (100 mg, 1.032 mmol, 1.5 equiv) was added and the suspension was then cooled to −20° C. with a NaCl-ice bath. To the reaction mixture was then added dropwise i-PrMgCl (1.376 mL, 2.752 mmol, 4.0 equiv) and the reaction was stirred for 10 minutes prior to be quenched with saturated NH₄Cl_{aq} solution (10 mL). Then, the mixture was extracted with pentane (2×30 mL). The combined organic layers were washed with brine (10 mL), and dried over anhydrous Na₂SO₄. After filtration and evaporation of the solvents, the residue was purified by flash chromatography (silica gel, 80/20 petroleum ether/EtOAc) to give the Weinreb amide R-As-2 as a yellow oil in 93% yield (200 mg). Rf=0.34 (80/20 petroleum ether/EtOAc); ¹H NMR (CDCl₃, 400 MHz, 23° C.) δ=6.99 (dt, J=15.2, 7.2 Hz, 1H); 6.46 (d, J=15.2 Hz, 1H); 5.65 (m, 1H); 5.49 (m, 1H); 4.22-4.181 (m, 1H); 3.71 (s, 3H); 3.26 (s, 3H); 2.49-2.38 (m, 2H); 1.70 (d, J=6.4 Hz, 3H); 0.91 (s, 9H); 0.067 (d, J=8.8 Hz, 6H) ppm. ¹³C NMR (CDCl₃, 100 MHz, 25° C.) δ=166.7, 144.1, 133.8, 125.7, 120.6, 77.2, 72.6, 61.6, 41.8, 32.3, 25.9, 18.1, 17.5, −4.3, −4.7 ppm.

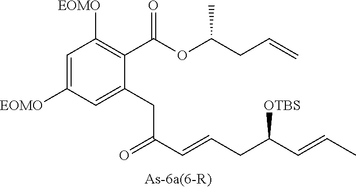

### Synthesis of the Acylated Compound As-6a(6-R):

A solution of ester As-1a (79 mg, 0.224 mmol, 1.5 equiv) in anhydrous THF (1.5 mL) at −78° C. was treated with freshly prepared LDA (0.352 M, 0.352 mmol, 2.2 equiv) via cannula. After 25 minutes, a solution of Weinreb amide R-As-2 (45 mg, 0.144 mmol, 1.0 equiv) in THF (1.5 mL) at −78° C. was added by syringe. The resulting mixture was then stirred for 20 minutes and the reaction was quenched by addition of saturated NH₄Cl_{aq} solution. Upon warming to 23° C., the reaction mixture was extracted with EtOAc (2×30 mL) and the combined organic layers were washed with brine (30 mL), dried over Na₂SO₄, filtered and concentrated. The residue was purified by flash chromatography (90/10 petroleum ether/EtOAc) to give As-6a(6-R) as a yellow oil in 52% yield (43 mg). R_{f}=0.48 (80/20 petroleum ether/EtOAc); ¹H NMR (CDCl₃, 400 MHz, 23° C.) δ=6.93-6.86 (m, 1H); 6.84 (d, J=2.0 Hz, 1H), 6.56 (d, J=2.0 Hz, 1H); 6.21 (d, J=16 Hz, 1H); 5.90-5.80 (m, 1H); 5.64-5.55 (m, 1H); 5.45 (dd, J=15.2, 6.4 Hz, 1H); 5.22 (s, 2H); 5.21 (s, 2H); 5.19-5.08 (m, 3H); 4.20-4.15 (m, 1H); 3.94 (d, J=16.4 Hz, 1H); 3.86 (d, J=16.4 Hz, 1H); 3.75-3.71 (m, 4H); 2.49-2.31 (m, 4H); 1.70 (d, J=6.0 Hz, 3H); 1.32-1.21 (m, 6H); 0.90 (s, 9H); 0.05 (d, J=5.2 Hz, 6H) ppm. ¹³C NMR (CDCl₃, 100 MHz, 25° C.) δ=195.8, 167.1, 159.1, 156.3, 144.4, 134.9, 133.9, 133.7, 131.1, 126.0, 118.7, 117.6, 111.2, 102.5, 93.5, 93.0, 72.5, 71.0, 64.3, 53.4, 45.3, 41.7, 40.2, 29.7, 25.8 (×3), 19.4, 18.2, 17.5, 15.1, −4.26, −4.78 ppm.

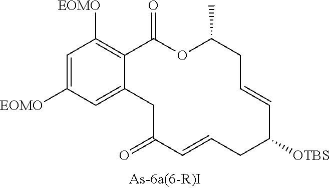

### Synthesis of Compound As-6a(6-R)I:

To a solution of As-6a(6-R) (140 mg, 0.231 mmol, 1.0 equiv) in toluene (10.0 mL) was degassed for 20 minutes and heated to 80° C. Grubb's II catalyst (10.0 mg, 11 μmol, 0.05 equiv) was added and stirred at 80° C. for 5 hours. After cooling down to 23° C., the reaction was treated with DMSO (57 μL, 60 equiv to catalyst) for 12 hours. The mixture was then passed through silica pad, and washed with petroleum ether/EtOAc 1:1 and then 1:2. The combined filtrate were concentrated under reduced pressure and the residue was purified by flash chromatography (90/10 petroleum ether/EtOAc) to afford As-6a(6-R)I in 65% yield (84 mg) R_{f}=0.28 (80/20 petroleum ether/EtOAc)¹H (CDCl₃, 400 MHz, 23° C.) δ=6.71 (s, 1H), 6.54-6.49 (m, 2H), 5.92 (d, J=16.0 Hz, 1H), 5.41-5.28 (m, 2H), 5.15 (s, 2H), 5.13-5.12 (m, 3H), 4.01-3.99 (m, 1H); 3.96 (d, J=14.8 Hz, 1H), 3.70-3.62 (m, 4H), 3.46 (d, J=14.8 Hz, 1H), 2.40-2.21 (m, 4H), 1.37 (d, J=6.0 Hz, 3H), 1.20-1.13 (m, 6H), 0.81 (s, 9H), −0.00 (s, 3H), −0.02 (s, 3H) ppm. ¹³C NMR (CDCl₃, 100 MHz, 25° C.) δ=197.5, 167.7, 159.1, 156.2, 145.7, 136.1, 134.9, 131.2, 127.7, 118.3, 109.5, 102.2, 93.4, 93.0, 73.1, 71.1, 64.5, 64.4, 44.2, 40.9, 39.4, 25.8 (×3), 20.2, 18.1, 15.0 (×2), −4.37, −4.75 ppm.

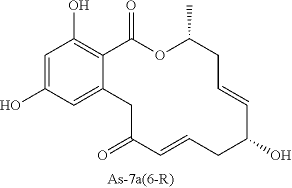 [SMILES: C[C@@H]1C/C=C/[C@H](O)C/C=C/C(=O)Cc2cc(O)cc(O)c2C(=O)O1]

### Synthesis of As-7a(6-R).

To a solution of As-6a(6-R)I (10.0 mg, 0.017 mmol, 1.0 equiv) in isopropanol (1.0 mL), sulfonic acid resin (33.6 mg, 0.102 mmol, 6.0 equiv, 3.0 mmol/g) was added and the suspension was heated at 40° C. for 16 hours. The reaction mixture was then filtered and the resin was rinsed with CH₂Cl₂ (5.0 mL) and MeOH (5.0 mL). After removal of solvents under reduced pressure, the residue was purified by prep. TLC to afford As-7a(6-R) in a 55% yield (3.1 mg) R_{f}=0.22 (60/40 EtOAc/petroleum ether)¹H (CDCl₃, 400 MHz, 23° C.) δ=11.2 (s, 1H), 9.07 (s, 1H), 6.65-6.57 (m, 1H), 6.19-6.17 (m, 2H), 5.77 (d, J=16.0 Hz, 1H), 5.43-5.36 (m, 1H), 5.28-5.21 (m, 2H), 4.29-4.28 (m, 1H), 3.92 (d, J=16.8 Hz, 1H), 3.86 (d, J=16.8 Hz, 1H), 2.53 (ddd, J=11.6, 7.2, 4 Hz, 1H), 2.38-2.32 (m, 1H), 2.25-2.22 (m, 1H), 2.17-2.09 (m, 1H), 1.16 (d, J=6.4 Hz, 3H) ppm, one OH signal is not visible. ¹³C NMR (CDCl₃, 100 MHz, 25° C.) δ=196.7, 176.0, 165.8, 163.0, 144.9, 141.2, 137.6, 132.5, 125.1, 113.2, 106.6, 102.7, 72.8, 72.1, 48.3, 40.7, 37.1, 18.6 ppm.

### General Procedure for the Synthesis of Hydroxyl Amine As-8:

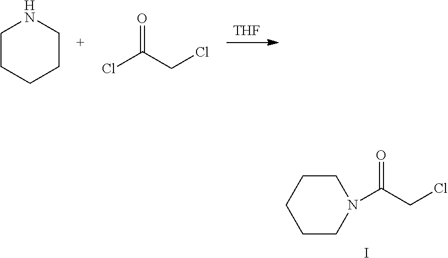

### Synthesis of Compound I:

To a solution of piperidine (51.9 ml, 525 mmol, 2.1 equiv) in THF (1000 mL) was added chloroacetic chloride (19.9 ml, 250 mmol, 1.0 equiv) slowly at 0° C. under nitrogen atmosphere. The reaction was allowed to warm to 23° C., and stirred for 1 hour. The reaction mixture was extracted with EA (2×500 ml) from sat. NH₄Cl solution, the combined organic layer was washed by brine (300 mL), dried over anhydrous Na₂SO₄. After filtration and evaporation under reduced pressure, the crude product (36.4 g, 90%) obtained was used directly for the next step without further purification. ¹H NMR (CDCl₃, 400 MHz, 25° C.) δ 4.06 (s, 2H), 3.54 (t, J=5.5 Hz, 2H), 3.43 (t, J=5.2 Hz, 2H), 1.70-1.60 (m, 4H), 1.60-1.51 (m, 2H) ppm. ¹³C NMR (CDCl₃, 100 MHz, 25° C.) 164.9, 47.5, 43.4, 41.3, 26.4, 25.5, 24.4 ppm.

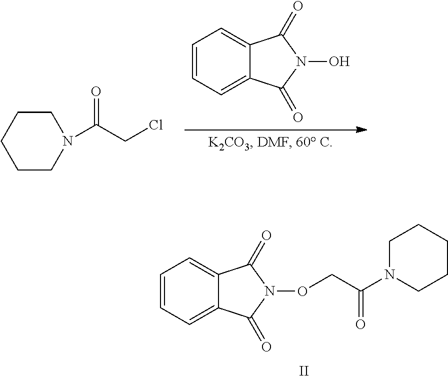

### Synthesis of Compound II:

To the suspension of N-hydroxyphthalimide (44.0 g, 270 mmol, 1.2 equiv) in DMF (600 mL), K₂CO₃ (46.6 g, 337 mmol, 1.5 equiv) was added in portion, which formed a large amount of solid. Then solution of I (36.4 g, 225 mmol, 1.0 equiv) in DMF (50 mL) was added by syringe. The reaction was heated to 60-65° C. for 3 hours. The solvent was evaporated under reduced pressure, and the residue obtained underwent extraction from CH₂Cl₂ and sat. NH₄Cl solution, the combined organic layer was washed by brine, dried over Na₂SO₄. The desired product precipitated during the evaporation, after filtration, gave II as the white solid in 70% yield. ¹H NMR (CDCl₃, 400 MHz, 25° C.) δ 7.86-7.71 (m, 4H), 4.84 (s, 2H), 3.63 (t, J=5.3 Hz, 2H), 3.57 (t, J=5.4 Hz, 2H), 1.74-1.65 (m, 4H), 1.65-1.57 (m, 2H) ppm. ¹³C NMR (CDCl₃, 100 MHz, 25° C.) 164.0, 163.0 (×2), 134.7 (×2), 128.8 (×2), 123.7 (×2), 75.9, 46.9, 43.1, 26.3, 25.3, 24.5 ppm.

### Synthesis of Hydroxyl Amine As-8

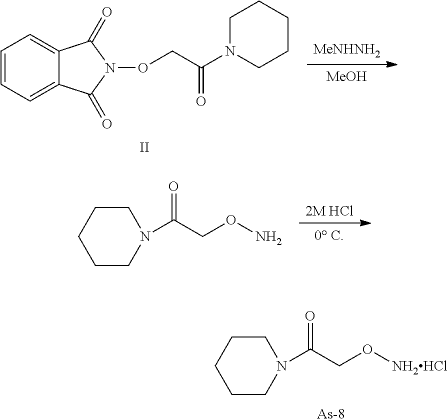

To the suspension of II (38.9 g, 135 mmol, 1.0 equiv) in MeOH (270 mL) was added MeNHNH₂ (7.55 mL, 142 mmol, 1.05 equiv) by syringe at 0° C. The reaction was allowed to warm to 23° C. and stirred for 1 hour. The solvent was evaporated under reduced pressure, water (150 mL) was added and the solid filtered. After removal of water, the crude product was redissolved in MeOH (150 mL), concentrated HCl (25.0 ml, 12 mmol/mL, 2.0 equiv) was added dropwise at 0° C. After stirring for 1 hour, the solvent was evaporated and the residue obtained was recrystallized from MeOH and ether to give the white solid in 73% yield (20.2 g). ¹H NMR (CDCl₃, 400 MHz, 25° C.) δ 4.89 (bs, 4H), 3.60 (t, J=5.4 Hz, 2H), 3.34 (t, J=5.4 Hz, 2H), 1.77-1.68 (m, 2H), 1.68-1.56 (m, 4H) ppm. ¹³C NMR (CD₃OD, 100 MHz, 25° C.) 167.4, 71.5, 46.6, 44.0, 27.3, 26.6, 25.3 ppm.

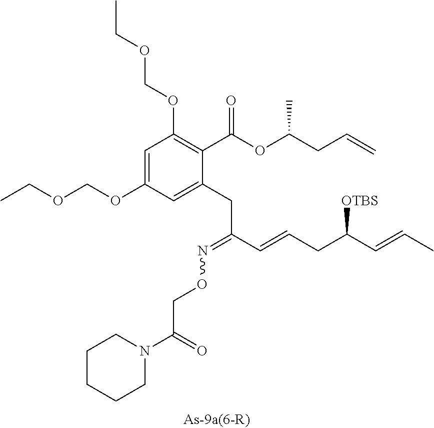 [SMILES: C=CC[C@@H](C)OC(=O)c1c(CC(/C=C/C[C@H](/C=C/C)O[Si](C)(C)C(C)(C)C)=NOCC(=O)N2CCCCC2)cc(OCOCC)cc1OCOCC]

### Synthesis of the Oxime As-9a(6-R).

To a stirred solution of As-6a(6-R) (53 mg, 0.090 mmol, 1.0 equiv) in pyridine (3.0 mL), at 45° C. under N₂, was added hydroxylamine hydrochloride As-8 (53 mg, 0.270 mmol, 3.0 equiv) in two portions over 48 hours. After that time the pyridine was evaporated and then the residue was dissolved in CH₂Cl₂ (30 mL), washed with saturated NH₄Cl_{aq} (10.0 mL) and dried over anhydrous Na₂SO₄. Filtration and evaporation of the solvents under reduced pressure, followed by flash chromatography (silica gel, gradient from 60/40 to 50/50, petroleum ether/EtOAc), afforded the desired compound As-9a(6-R) (colorless oil) in 75% yield (50 mg) as a mixture of E/Z isomers in a 1:1 ratio. R_{f}=0.36 (50/50 petroleum ether/EtOAc); ¹H NMR (CDCl₃, 400 MHz, 23° C.) δ=6.79-6.75 (m, 3H); 6.56 (d, J=2.0 Hz, 1H); 6.44 (d, J=2.0 Hz, 1H); 6.19-6.12 (m, 2H); 6.07-6.00 (m, 1H); 5.91-5.81 (m, 2H); 5.26-5.21 (m, 6H); 5.21-5.12 (m, 12H); 4.78 (s, 2H); 4.75 (s, 2H); 4.06-4.01 (m, 2H); 3.88 (s, 4H); 3.76-3.66 (m, 8H); 3.60-3.54 (m, 4H); 3.45 (m, 2H); 3.31-3.29 (m, 2H); 2.42-2.21 (m, 8H); 1.63-1.44 (m, 20H); 1.37-1.34 (m, 6H); 1.26-1.20 (m, 12H); 0.85 (s, 18H); −0.02 (m, 12H) ppm. ¹³C NMR (CDCl₃, 100 MHz, 25° C.) δ=167.28, 167.24, 167.0, 166.7, 159.1, 158.8, 157.2, 155.55, 155.51, 154.5, 138.2, 137.6, 136.1, 134.2, 133.9, 133.8, 133.77, 133.75, 128.1, 125.4, 120.5 (×2), 118.4, 118.3, 117.8, 117.7, 109.3, 109.03, 101.5, 101.4, 93.4 (×2), 93.2, 93.1, 73.2, 73.0, 72.9, 72.5, 71.0 (×2), 64.3 (×2), 64.2 (×2), 46.1, 46.0, 42.9, 42.7, 42.4 (×2), 40.2 (×2), 34.1 (×2), 28.0 (×2), 26.4, 26.3, 25.5 (×3), 25.4 (×3), 24.6, 24.5, 19.6, 19.5, 18.2, 18.1, 17.5, 17.3, 15.1 (×2), 15.0 (×2), −4.38, −4.40, −4.81 (×2) ppm.

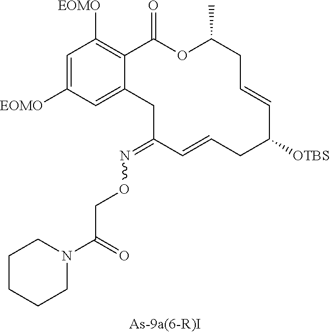

### Synthesis of the Protected Pochoxime As-9a(6-R)I:

A solution of As-9a(6-R) (9.40 g, 12.8 mmol) in toluene (450 mL) was degassed for 20 min under nitrogen atmosphere and then heated to 80° C. Grubbs' II catalyst (545 mg, 0.64 mmol, 0.05 equiv) was added and stirred at 80° C. for 5 hours. After cooling down to 23° C., the reaction was treated with DMSO (3.0 mL, 60 equiv to catalyst) for 24 hours. The mixture was passed through silica pad, and washed first with petroleum ether/EtOAc 50/50, and then 1/2. The combined filtrates were concentrated under reduced pressure and the residue was purified by flash chromatography (petroleum ether/EtOAc, 75/25) to afford As-9a(6-R)I (9.0 g, quantitative) as a mixture of E/Z isomers in the ratio of 1/1. ¹H NMR (CDCl₃, 400 MHz, 25° C.) δ 6.78 (d, J=17.6 Hz, 1H), 6.70 (s, J=1.6 Hz, 1H), 6.69 (d, J=1.6 Hz, 1H), 6.62 (d, J=1.6 Hz, 1H), 6.53 (d, J=1.6 Hz, 1H), 6.08 (d, J=16.8 Hz, 1H), 5.79-5.70 (m, 2H), 5.36-5.34 (m, 4H), 5.18-5.05 (m, 10H), 4.80 (s×2, 4H), 3.86-3.83 (m, 4H), 3.72-3.66 (m, 12H), 3.58 (m, 2H), 3.49 (m, 2H); 3.43-3.38 (m, 4H), 2.45-2.13 (m, 8H), 1.63-1.55 (m, 12H), 1.41 (d×2, J=6.4 Hz, 6H), 1.23-1.17 (m, 12H), 0.84 (2×s, 18H), 0.01 (s×2, 6H), −0.01 (s×2, 6H) ppm.

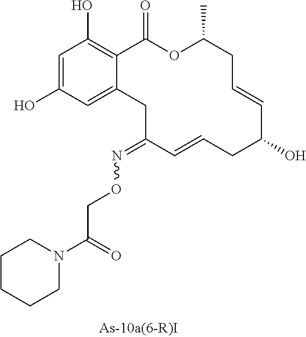 [SMILES: C[C@@H]1C/C=C/[C@H](O)C/C=C/C(=NOCC(=O)N2CCCCC2)Cc2cc(O)cc(O)c2C(=O)O1]

### Synthesis of Pochoxime As-10a(6-R)I:

A solution of As-9a(6-R)I (140 mg, 0.2 mmol) in THF (3.0 mL) was treated with a solution of TBAF in THF (0.3 mL, 1M in THF, 1.5 equiv) at 0° C. The reaction was allowed to reach room temperature and was stirred for 3 hours. Then the mixture was extracted from sat. NH₄Cl_{aq} solution with EtOAc (3×10 mL), washed with brine (15 mL), dried over anhydrous Na₂SO₄, and concentrated. Purification by flash chromatography column (EtOAc) afforded the corresponding TBS deprotected alcohol in 88% yield (104 mg). To a solution of this compound (100 mg) were added 10 equiv of sulfonic acid polystyrene resin (sulfonic acid resin MP, 70-90 mesh, 3.0 mmol/g, Novabiochem, 01-64-0432) and the suspension was stirred for 4 hours at room temperature. The mixture was then filtered, and passed through a pad of silica gel. The two isomers were separated on an HPLC (20-80% CH3CN in Water gradient in 50 min, flow: 2 mL/min, Discovery® HS C18, 5 μm, 5 cm×10.0 mm). ¹H (Z-isomer, CDCl₃, 400 MHz, 25° C.) δ 10.4 (s, 1H), 6.67 (d, J=14.3 Hz, 1H), 6.55 (d, J=2.3 Hz, 1H), 6.31 (d, J=2.4 Hz, 1H), 5.90-5.81 (m, 1H), 5.66 (dt, J=16.0, 7.9 Hz, 1H), 5.54-5.39 (m, 2H), 4.78 (s, 2H), 4.17 (m, 2H), 3.82 (d, J=14.6 Hz, 1H), 3.56-3.51 (m, 2H), 3.40-3.38 (m, 2H), 2.47-2.41 (m, 2H), 2.35-2.29 (m, 2H), 1.64-1.56 (m, 6H), 1.46 (d, J=2.4 Hz, 3H) ppm, 2 OH signals are not visible. ¹H (E-isomer, CDCl₃, 400 MHz, 25° C.) δ 6.98 (d, J=2.49 Hz, 1H), 6.30 (d, J=2.5 Hz, 1H), 5.95 (d, J=16.1 Hz, 1H), 5.85-5.77 (m, 1H), 5.67 (dt, J=16.0, 7.9 Hz, 1H), 5.32-5.43 (m, 2H), 4.79 (d, J=3.9 Hz, 2H), 4.17 (m, 2H), 3.82 (d, J=14.6 Hz, 1H), 3.56-3.51 (m, 2H), 3.40-3.38 (m, 2H), 2.47-2.41 (m, 2H), 2.35-2.29 (m, 2H), 1.64-1.56 (m, 6H), 1.46 (d, J=2.4 Hz, 3H) ppm, 3 OH signals are not visible.

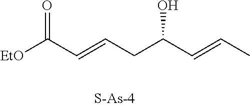

### Synthesis of the Enantiomerically Pure Aldol Adduct S-As-4.

A flame dried 25 mL two necked round bottomed flask, was charged with (S,S)-Denmark catalyst (8.5 mg, 0.01 mmol, 0.01%), crotonaldehyde (85 μl, 1.0 mmol, 1.0 equiv), TBAI (73 mg, 0.2 mmol, 20%) and 5.0 mL of dry CH₂Cl₂. This solution was cooled down to −78° C. with a dry ice-acetone bath. After 5 minutes of stirring SiCl₄ (123 μl, 1.1 mmol, 1.1 equiv) was added dropwise, and the solution was stirred for additional 5 minutes. To this solution, neat silylketene acetal As-3 (458 mg, 2.0 mmol, 2.0 equiv) was added in a dropwise manner over 5 minutes period. After 30 minutes, Et₃N (459 μl, 3.3 mmol. 3.3 equiv) in 2.0 mL of dry methanol was added by canula at −78° C., the mixture was stirred for 1 hour, then quenched with saturated NH₄Cl_{aq} (10 mL), and kept stirred for one hour more. The mixture was then extracted with CH₂Cl₂ (3×30 mL) and the combined organic phase were dried with Na₂SO₄. Filtration and evaporation of the solvents under reduced pressure followed by flash chromatography (silica gel, 80/20 petroleum ether/EtOAc) afforded the desired compound S-As-4 as a yellow oil in 52% yield (103 mg). Rf=0.18 (80/20 petroleum ether/EtOAc); ¹H (CDCl₃, 400 MHz, 23° C.) δ=6.99 (dt, J=15.0, 7.2 Hz, 1H); 6.46 (dd, J=15.0, 1.6 Hz, 1H); 5.76 (m, 1H); 5.56 (m, 1H); 4.25-4.11 (m, 3H); 2.46-2.43 (m, 2H); 1.73 (d, J=6.4 Hz, 3H); 1.32 (t, J=7.2 Hz, 3H) ppm, OH signal is not visible. ¹³C NMR (CDCl₃, 100 MHz, 25° C.) δ=166.3, 144.7, 132.9, 127.7, 123.8, 71.5, 60.2, 40.1, 17.6, 14.2 ppm.

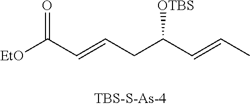 [SMILES: C/C=C/[C@H](C/C=C/C(=O)OCC)O[Si](C)(C)C(C)(C)C]

### Synthesis of Protected Aldol Adduct TBS-S-As-4.

To a solution of S-As-4 (173.5 mg, 0.867 mmol, 1.0 equiv) in dry DMF (5.0 mL), was added imidazole (94.45 mg, 1.387 mmol, 1.5 equiv) at room temperature. After 5 minutes of stirring TBSCl (196 mg, 1.3 mmol, 1.3 equiv) was added. The reaction mixture was stirred at the same temperature for 16 hours. After evaporation of the solvent, the residue was purified by flash chromatography (silica gel, 90/10 petroleum ether/EtOAc) to give TBS-S-As-4 in 97% yield (252 mg). Rf=0.82 (80/20 petroleum ether/EtOAc); ¹H NMR (CDCl₃, 400 MHz, 23° C.) δ=6.99 (dt, J=15.0, 7.2 Hz, 1H); 6.46 (dd, J=15.0 Hz, 1H); 5.76 (m, 1H); 5.56 (m, 1H); 4.25-4.11 (m, 3H); 2.46-2.43 (m, 2H); 1.73 (d, J=6.4 Hz, 3H); 1.32 (t, J=7.2 Hz, 3H); 0.91 (s, 9H); 0.06 (d, J=7.6 Hz, 6H) ppm. ¹³C NMR (CDCl₃, 100 MHz, 25° C.) δ=166.4, 145.7, 133.7, 125.8, 123.1, 72.5, 60.1, 41.4, 25.7, 18.1, 17.5, 14.2, −2.9, −4.3, −4.8 ppm.

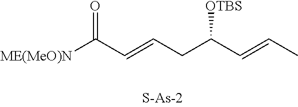

### Synthesis of Weinreb Amide S-As-2.

To a solution of TBS-S-As-2 (252 mg, 0.846 mmol, 1.0 equiv) in dry THF (5.0 mL), was added N,O-dimethylhydroxylamine hydrochloride salt (123.7 mg, 1.268 mmol, 1.5 equiv) this suspension was then cooled to −20° C. with a NaCl-ice bath. To the reaction mixture was then added dropwise i-PrMgCl (1.692 mL, 3.384 mmol, 4.0 equiv) and the reaction was stirred for 10 minutes prior to quench with saturated NH₄Cl_{aq} (10 mL). Then, the reaction was extracted with pentane (2×30 mL). The combined organic layers were washed with brine (10 mL), and dried over anhydrous Na₂SO₄. After filtration and evaporation of the volatiles, the residue was purified by flash chromatography (silica gel, 80/20 petroleum ether/EtOAc) to give the weinreb amide S-As-2 as a yellow oil in 82% yield (216 mg). Rf=0.34 (80/20 petroleum ether/EtOAc); ¹H NMR (CDCl₃, 400 MHz, 23° C.) δ=6.99 (dt, J=15.2, 7.2 Hz, 1H); 6.46 (d, J=15.2 Hz, 1H); 5.65 (m, 1H); 5.49 (m, 1H); 4.22-4.181 (m, 1H); 3.71 (s, 3H); 3.26 (s, 3H); 2.49-2.38 (m, 2H); 1.70 (d, J=6.4 Hz, 3H); 0.91 (s, 9H); 0.07 (d, J=8.8 Hz, 6H) ppm. ¹³C NMR (CDCl₃, 100 MHz, 25° C.) δ=166.7, 144.1, 133.8, 125.7, 120.6, 77.2, 72.6, 61.6, 41.8, 32.3, 25.9, 18.1, 17.5, −4.3, −4.7 ppm.

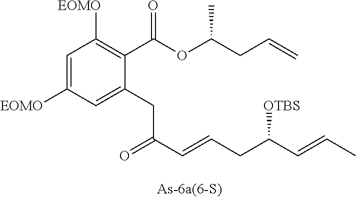

### Synthesis of Compound As-6a(6-S).

A solution of ester As-1a (84 mg, 0.234 mmol, 1.5 equiv) in anhydrous THF (1.5 mL) at −78° C. was treated with freshly prepared LDA (0.352 M, 0.352 mmol, 2.2 equiv) via canula. After 30 minutes, a solution of Weinreb amide S-As-2 (50 mg, 0.160 mmol, 1 equiv) in THF (1.5 mL) at −78° C. was added by syringe. The resulting mixture was then stirred for 25 minutes and the reaction was quenched by addition of sat. NH₄Cl_{aq} solution. Upon warming to 23° C., the reaction mixture was extracted with EtOAc (2×30 mL), and the combined organic layers were washed with brine (30 mL), dried over Na₂SO₄, filtered and concentrated. The residue was purified by flash chromatography (90:10 petroleum ether/EtOAc) to give As-6a(6-S) as a yellow oil in 69% yield (64.5 mg). R_{f}=0.48 (80/20 petroleum ether/EtOAc); ¹H NMR (CDCl₃, 400 MHz, 23° C.) δ=6.93-6.84 (m, 2H); 6.52 (s, 1H); 6.21 (d, J=16 Hz, 1H); 5.9-5.8 (m, 1H); 5.6-5.4 (m, 1H); 5.4-5.3 (m, 1H); 5.3 (s, 4H); 5.3-5.1 (m, 3H); 4.2-4.1 (m, 1H); 3.9-3.7 (m, 2H); 3.8-3.7 (m, 4H); 2.5-2.3 (m, 4H); 1.7 (d, J=6.4 Hz, 3H); 1.32-1.19 (m, 10H); 0.89 (s, 9H); 0.05 (d, J=5.2 Hz, 6H) ppm. ¹³C NMR (CDCl₃, 100 MHz, 23° C.) δ=195.8, 167.1, 159.0, 156.3, 144.5, 134.9, 133.9, 133.8, 133.6, 131.1, 125.9, 117.5, 111.2, 102.5, 93.5, 93.0, 72.5, 71.0, 64.4, 64.3, 45.3, 41.7, 40.2, 25.8, 19.4, 18.1, 17.5, 15.1, 15.0, 14.9, −4.30, −4.34, −4.8 ppm.

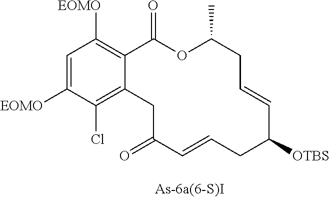

### Synthesis of Compound As-6a(6-S)I:

A solution of As-6a(6-S) (28.4 mg, 0.048 mmol, 1.0 equiv) in toluene (1.0 mL) was degassed for 20 min and heated to 80° C. Grubb's II catalyst (2.0 mg, 24 □mol, 0.05 equiv) was added and stirred at 80° C. for 5 hours. After cooling down to 23° C., the reaction was treated with DMSO (11 μL, 60 equiv to catalyst) for 24 hours. The mixture was passed through silica pad, and washed with 50/50 petroleum ether/EtOAc and then 25/75. The combined filtrate were concentrated under reduced pressure and the residue was purified by flash chromatography (90/10 petroleum ether/EtOAc) to afford As-6a(6-S)I in 44.4% yield (10.2 mg). R_{f}=0.28 (80/20 petroleum ether/EtOAc) ¹H (CDCl₃, 400 MHz, 23° C.) δ=6.77 (s, 1H), 6.76-6.72 (m, 1H), 6.60 (s, 1H), 5.96 (d, J=16.0 Hz, 1H), 5.66-5.61 (m, 1H), 5.47-5.42 (m, 1H), 5.23-5.19 (m, 5H), 4.38-4.37 (m, 1H), 4.08 (d, J=14.8 Hz, 1H), 3.77-3.69 (m, 4H), 3.51 (d, J=14.8 Hz, 1H), 2.45-2.30 (m, 4H), 1.43 (d, J=6.0 Hz, 3H), 1.28-1.21 (m, 6H), 0.89 (s, 9H), 0.05 (s, 6H) ppm. ¹³C NMR (CDCl₃, 100 MHz, 25° C.) δ=197.5, 167.7, 159.1, 156.2, 145.7, 136.1, 134.9, 131.2, 127.7, 118.3, 109.5, 102.2, 93.4, 93.0, 73.1, 71.1, 64.5, 64.4, 44.2, 40.9, 39.4, 25.8 (×3), 20.2, 18.1, 15.0 (×2), −4.37, −4.75 ppm.

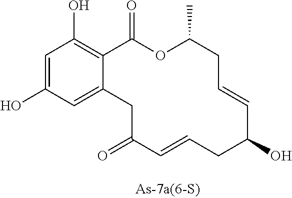 [SMILES: C[C@@H]1C/C=C/[C@@H](O)C/C=C/C(=O)Cc2cc(O)cc(O)c2C(=O)O1]

### Synthesis of As-7a(6-S).

To a solution of As-6a(6-S)I (12.2 mg, 0.021 mmol, 1.0 equiv) in isopropanol (1.0 mL), sulfonic acid resin (41 mg, 0.126 mmol, 6.0 equiv, 3.0 mmol/g) was added and the suspension was heated at 40° C. for 16 hours. The reaction mixture was filtered and the resin rinsed with CH₂Cl₂ (5.0 mL) and MeOH (5.0 mL). After removal of solvents under reduced pressure, the residue was purified by prep. TLC to afford As-7a(6-S) in 57% yield (5.1 mg,) R_{f}=0.24 (40/60 petroleum ether/EtOAc) ¹H (CDCl₃, 400 MHz, 25° C.) δ=11.4 (s, 1H), 9.13 (s, 1H), 6.58-6.50 (m, 1H), 6.21-6.19 (m, 2H), 5.73 (d, J=15.6 Hz, 1H), 5.39-5.33 (m, 1H), 5.21-5.13 (m, 2H), 4.15-4.10 (m, 1H), 4.01 (d, J=16.8 Hz, 1H), 3.55 (d, J=16.8 Hz, 1H), 2.60-2.53 (m, 1H), 2.41-2.35 (m, 1H), 2.21-2.18 (m, 1H), 2.17-2.14 (m, 1H), 1.18 (d, J=6.4 Hz, 3H) ppm. ¹³C NMR (CDCl₃, 100 MHz, 23° C.) δ=196.6, 171.2, 166.4, 163.2, 144.3, 141.2, 137.6, 131.7, 125.7, 113.1, 106.3, 102.9, 73.2, 72.5, 49.0, 40.8, 37.1, 18.2 ppm.

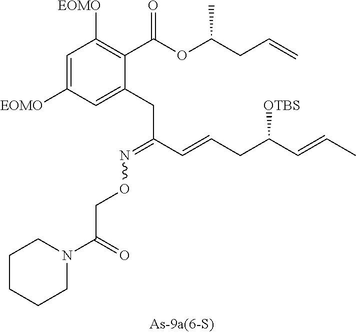

### Synthesis of the Open Chain Oxime As-9a(6-S).

To a stirred solution of the As-6a(6-S) (64 mg, 0.108 mmol, 1.0 equiv) in pyridine (5.0 mL), at 45° C. under N₂, was added hydroxylamine hydrochloride As-8 (64 mg, 0.233 mmol, 2.0 equiv) in two portions over 24 hours. After evaporation of the pyridine, the residue was dissolved in CH₂Cl₂ (30 mL), washed with sat. NH₄Cl_{aq} (10 mL) and dried over anhydrous Na₂SO₄. Filtration and evaporation of the solvents under reduced pressure, followed by flash chromatography (silica gel, gradient from 60/40 to 50/50, petroleum ether/EtOAc) afforded the desired compound As-9a(6-S) (colorless oil) in 75% yield (56 mg) as a mixture of E/Z isomers in the ratio of 1/1. Rf=0.36 (50/50 petroleum ether/EtOAc); ¹H NMR (CDCl₃, 400 MHz, 23° C.) δ=6.79-6.78 (m, 3H); 6.55 (d, J=2.0 Hz, 1H); 6.44 (d, J=2.0 Hz, 1H); 6.19-6.11 (m, 2H); 6.07-5.09 (m, 1H); 5.91-5.81 (m, 2H); 5.50-5.23 (m, 6H); 5.21-5.10 (m, 11H); 4.78 (d, J=12.4 Hz, 4H); 4.06-4.01 (m, 2H); 3.9-3.82 (m, 4H); 3.76-3.66 (m, 8H); 3.60-3.53 (m, 4H); 3.45-3.43 (m, 2H); 3.31-3.29 (m, 2H); 2.53-2.23 (m, 8H); 1.63-1.44 (m, 20H); 1.37-1.20 (m, 18H); 0.85 (s, 18H); −0.01-0.03 (m, 12H) ppm. ¹³C NMR (CDCl₃, 100 MHz, 23° C.) δ=167.2, 166.9, 166.7 (×2), 159.1, 158.8, 157.2, 155.5, 154.5, 154.2, 138.2, 137.6, 136.1, 134.1, 133.9, 133.8, 133.75, 133.72, 128.1, 125.4, 120.5 (×2), 117.75, 117.7, 109.3, 109.0, 101.5, 101.4, 93.4 (×2), 93.2, 93.1, 73.2, 72.9, 72.8, 72.5, 71.0, 70.9, 64.3 (×2), 64.2 (×2), 46.0, 45.9, 42.9, 42.8, 42.7, 42.3, 40.2 (×2), 34.1 (×2), 27.9 (×2), 26.4, 26.3, 25.8 (×3), 25.7 (×3), 25.5, 25.4, 24.54, 24.5, 19.6, 19.5, 18.2, 18.1, 17.5, 17.4, 15.1 (×2), 15.0 (×2), −4.3, −4.4, −4.8 (×2) ppm.

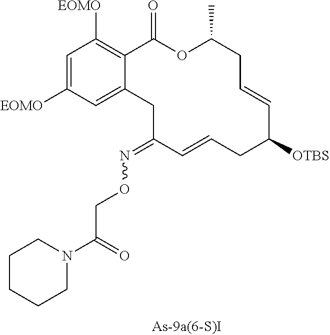

### Synthesis of the Protected Pochoxime As-9a(6-S)I:

A solution of the open chain oximes As-9a(6-S) (50 mg, 0.067 mmol, 1.0 equiv) in anhydrous CH₂Cl₂ (7.0 mL), under a steam of N₂, was heated to reflux. To this solution was added Grubbs II catalyst (0.2 mol %, 11.4 mg) in 1.0 mL of CH₂Cl₂, and the mixture was refluxed for 8 hours. After the solution was cooled to room temperature and then was filtered over a pad of celite to remove the catalyst. After evaporation of the solvent under reduced pressure, the residue was purified by flash chromatography (silica gel, 50/50 petroleum ether/EtOAc), to afford the desired compound As-9a(6-S)I (colorless oil) in 83% yield (39 mg) as a mixture of E/Z isomers in the ratio of 1/1. Rf=0.31 (60/40 EtOAc/petroleum ether); ¹H NMR (CDCl₃, 400 MHz, 23° C.) δ=6.73-6.69 (m, 4H); 6.06 (bs, 1H); 6.12-6.07 (m, 1H); 6.05-5.99 (m, 2H); 5.67-5.60 (m, 2H); 5.48 (bs, 1H); 5.44 (bs, 1H); 5.23 (bs, 8H); 4.86 (d, J=13.6 Hz, 2H); 4.81 (d, J=13.6 Hz, 2H); 4.36-4.32 (m, 2H); 3.75-3.70 (m, 8H); 3.65-3.56 (m, 4H); 3.48-3.38 (m, 4H); 2.46-2.24 (m, 8H); 1.67-1.59 (m, 12H); 1.46 (d, J=6.0 Hz, 6H); 1.30-1.20 (m, 20H); 0.88 (s, 18H); 0.042 (s, 12H) ppm.

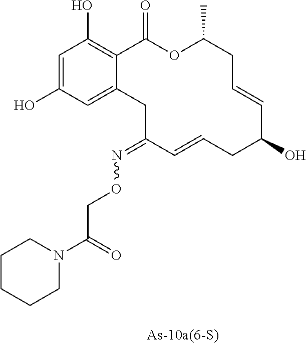 [SMILES: C[C@@H]1C/C=C/[C@@H](O)C/C=C/C(=NOCC(=O)N2CCCCC2)Cc2cc(O)cc(O)c2C(=O)O1]

### Synthesis of Pochoxime As-10a(6-S)

To a solution of As-9a(6-S)I (20.0 mg, 0.021 mmol, 1.0 equiv) in methanol (2.0 mL), sulfonic acid resin (111 mg, 0.34 mmol, 10 equiv, 3.0 mmol/g) was added and heated at room temperature for 16 hours. The reaction mixture was filtered and the resin was rinsed with CH₂Cl₂ (5.0 mL) and MeOH (5.0 mL). After removal of solvents under reduced pressure, the residue was purified by HPLC (20-80% CH₃CN in Water gradient in 50 min, flow: 2 mL/min, Discovery® HS C18, 5 μm, 5 cm×10.0 mm) to afford pochoximes As-10a(6-S) (Z-isomer: 2.4 mg; E-isomer: 2.8 mg). ¹H NMR (Z-isomer, MeOD-d4, 400 MHz) δ=6.70 (d, J=16.4 Hz, 1H), 6.28 (d, J=2 Hz, 1H), 6.15 (d, J=2.4 Hz, 1H), 6.13-6.04 (m, 1H), 5.71-5.64 (m, 1H), 5.64 (dd, J=15.6, 6.4 Hz, 1H), 5.33-5.27 (m, 1H), 4.79 (d, J=2.0 Hz, 2H), 4.14-4.13 (m, 1H), 4.02 (d, J=2.0 Hz, 1H), 3.57-3.55 (m, 2H), 3.51-3.46 (m, 3H), 2.64-2.57 (m, 1H), 2.40-2.33 (m, 2H), 2.25-2.18 (m, 1H), 1.66-1.58 (m, 6H), 1.43 (d, J=6.0 Hz, 3H) ppm. ¹³C NMR (MeOD-d4, 100 MHz, 25° C.) δ=170.0, 169.7, 162.4, 157.2, 142.0, 140.0, 136.8, 126.9, 121.0, 108.9, 102.3, 72.8, 72.6, 72.4, 64.7, 47.2, 44.2, 40.5, 39.2, 35.2, 27.4, 26.7, 25.4, 25.2, 19.6 ppm. ¹H NMR (E-isomer, CDCl₃, 400 MHz) δ=6.22 (d, J=2.0 Hz, 1H), 6.19 (d, J=2.0 Hz, 1H), 6.15 (d, J=2.4 Hz, 1H), 6.05-5.99 (m, 1H), 5.91 (d, J=16.4 Hz, 1H), 5.71-5.65 (m, 1H), 5.46 (dd, J=15.6, 6.4 Hz, 1H), 5.35-5.31 (m, 1H), 4.35 (d, J=15.6 Hz, 1H), 4.16-4.15 (m, 1H), 3.83 (d, J=15.2 Hz, 1H), 3.61-3.51 (m, 4H), 2.68-2.61 (m, 1H), 2.44-2.37 (m, 2H), 2.22-2.17 (m, 1H), 1.71-1.70 (m, 1H), 1.64-1.60 (m, 6H), 1.45 (d, J=6.4 Hz, 3H) ppm. ¹³C NMR (MeOD, 100 MHz, 25° C.) δ=171.0, 169.4, 162.6, 159.7, 140.9, 137.0, 135.8, 127.8, 126.8, 109.6, 102.3, 72.8, 72.7, 72.74, 64.7, 47.3, 44.2, 40.3, 39.1, 30.2, 27.5, 26.7, 25.4, 25.2, 19.5 ppm.

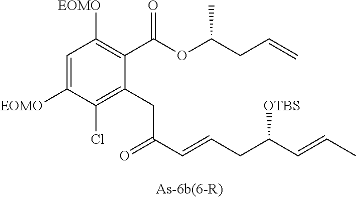

### Synthesis of Compound As-6b(6-R):

A solution of ester As-1b (144 mg, 0.372 mmol, 1.5 equiv) in anhydrous THF (2.0 mL) at −78° C. was treated with freshly prepared LDA (0.545 M, 0.545 mmol, 2.2 equiv) via cannula. After 25 minutes, a solution of Weinreb amide R-As-2 (70 mg, 0.224 mmol, 1.0 equiv) in THF (1.5 mL) at −78° C. was added by syringe. The resulting mixture was then stirred for 25 minutes and the reaction was quenched by addition of sat. NH₄Cl_{aq} solution. Upon warming to 23° C., the reaction mixture was extracted with EtOAc (2×30 mL), and the combined organic layers were washed with brine (30 mL), dried over Na₂SO₄, filtered and concentrated. The residue was purified by flash chromatography (90/10 petroleum ether/EtOAc) to give As-6b(6-R) as a yellow oil in 64% yield (89 mg). R_{f}=0.48 (70/30 petroleum ether/EtOAc); ¹H NMR (CDCl₃, 400 MHz, 23° C.) δ=7.08 (s, 1H); 6.93-6.85 (m, 1H); 6.17 (d, J=15.6 Hz, 1H); 5.82-5.71 (m, 1H); 5.61-5.52 (m, 1H); 5.42 (dd, J=15.6, 6.4 Hz, 1H); 5.27 (s, 2H); 5.17 (s, 2H); 5.14-5.02 (m, 3H); 4.18-4.14 (m, 1H); 4.02 (s, 2H); 3.74-3.68 (m, 4H); 2.37-2.30 (m, 4H); 1.66 (d, J=6.4 Hz, 3H); 1.25-1.18 (m, 9H); 0.86 (s, 9H); 0.021 (d, J=8.4 Hz, 6H) ppm. ¹³C NMR (CDCl₃, 400 MHz, 25° C.) δ=194.3, 166.5, 154.5, 153.8, 144.4, 133.75, 133.72, 132.7, 131.0, 126.0, 120.4, 117.7, 117.6, 102.9, 93.9, 93.7, 72.5, 72.4, 64.6, 64.4, 42.7, 41.7, 40.1, 25.2 (×3), 19.4, 18.2, 17.5, 15.03, 15.0, −4.23, −4.76 ppm.

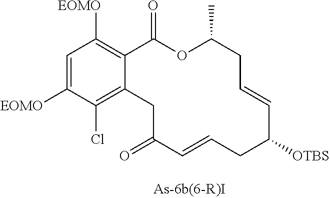

### Synthesis of Compound As-6b(6-R)I.

A solution of As-6b(6-R) (34 mg, 0.054 mmol, 1.0 equiv) in toluene (1.0 mL) was degassed for 20 min and heated to 80° C. Grubbs II catalyst (3.0 mg, 24 μmol, 0.05 equiv) was added and stirred at 80° C. for 5 hours. After cooling down to 23° C., the reaction was treated with DMSO (11 μL, 60 equiv. to catalyst) for 24 hours. The mixture was passed through silica pad, and washed with petroleum ether/ethyl acetate 50/50 and then 25/75. The combined filtrates were concentrated under reduced pressure and the residue was purified by flash chromatography (90/10 petroleum ether/EtOAc) to afford As-6b(6-R) I in 48% yield (15.4 mg) R_{f}=0.3 (80/20 petroleum ether/EtOAc)¹H (CDCl₃, 400 MHz, 23° C.) δ=7.11 (s, 1H), 6.77-6.69 (m, 1H), 5.87 (d, J=15.6 Hz, 1H); 5.47-5.39 (m, 1H), 5.34 (s, 2H), 5.33-5.31 (m, 1H), 5.26 (s, 2H), 5.18-5.14 (m, 1H), 4.32-4.28 (m, 1H), 4.03 (d, J=17.2 Hz, 1H), 3.86-3.67 (m, 5H), 2.36-2.25 (m, 4H), 1.36 (d, J=6.8 Hz, 3H), 1.30-1.23 (m, 6H), 0.90 (s, 9H), 0.07 (s, 3H), 0.04 (s, 3H) ppm. ^{D}C NMR (CDCl₃, 100 MHz, 23° C.) δ=195.6, 166.8, 154.6, 153.8, 143.6, 135.8, 132.8, 129.9, 126.2, 120.4, 117.8, 102.9, 93.9, 93.6, 72.6, 71.8, 64.8, 64.6, 44.6, 40.8, 39.0, 25.9 (×3), 19.7, 18.1, 15.0 (×2), −4.5, −4.8 ppm.

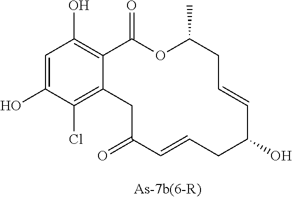 [SMILES: C[C@@H]1C/C=C/[C@H](O)C/C=C/C(=O)Cc2c(Cl)c(O)cc(O)c2C(=O)O1]

### Synthesis of As-7b(6-R).

To a solution of As-6b(6-R)I (11.2 mg, 0.0188 mmol, 1.0 equiv) in isopropanol (1.0 mL), sulfonic acid resin (37.0 mg, 0.1128 mmol, 6.0 equiv, 3.0 mmol/g) was added and the suspension was heated at 40° C. for 16 hours. The reaction mixture was filtered and the resin was rinsed with CH₂Cl₂ (5.0 mL) and MeOH (5.0 mL). After removal of solvents under reduced pressure, the residue was purified by prep. TLC (60/40 EtOAc/petroleum ether) to afford As-7b(6-R) in 47% yield (3.0 mg,) R_{f}=0.24 (60/40 EtOAc/petroleum ether); ¹H (CDCl₃, 400 MHz, 23° C.) δ=6.65-6.57 (m, 1H), 6.39 (s, 1H), 5.73 (d, J=15.2 Hz, 1H), 5.42-5.34 (m, 1H), 5.27-5.21 (m, 1H), 5.12-5.06 (m, 1H), 4.12-4.11 (m, 1H), 4.08 (d, J=18 Hz, 1H), 3.91 (d, J=18 Hz, 1H), 2.49-2.43 (m, 2H), 2.24-2.16 (m, 1H), 2.05-1.91 (m, 1H), 1.18 (d, J=6.4 Hz, 3H) ppm. ¹³C NMR (CDCl₃, 100 MHz, 25° C.) δ=198.7, 170.7, 163.1, 159.7, 145.2, 137.1, 131.7, 126.8, 116.6, 109.1, 104.0, 73.5, 73.2, 46.9, 41.0, 37.7, 30.7, 18.0 ppm.

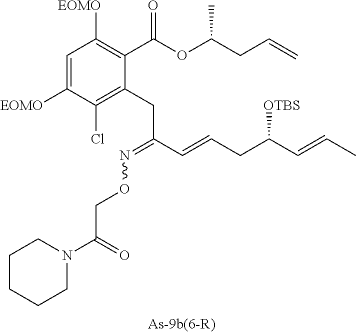

### Synthesis of the Open Chain Oxime As-9b(6-R):

To a stirred solution of the acylation product As-6b(6-R) (85 mg, 0.136 mmol) in pyridine (3.0 mL) at 45° C. under N₂, was added hydroxylamine hydrochloride As-8 (80 mg, 0.408 mmol) in two portions over 48 hours. After evaporation of the pyridine, the residue was dissolved in CH₂Cl₂ (30 mL) washed sat. NH₄Cl_{aq} (10 mL) and dried over anhydrous Na₂SO₄. Filtration and evaporation of the solvents under reduced pressure, followed by flash chromatography (silica gel, gradient from 60/40 to 50/50, petroleum ether/EtOAc), afforded the desired compound As-9b(6-R) (colorless oil) in 53% combined yield (41 mg). More polar isomer (21 mg, colorless oil) R_{f}=0.37 (50/50 petroleum ether/EtOAc); ¹H NMR (CDCl₃, 400 MHz, 23° C.) (Z)-Oxime δ=7.06 (s, 1H); 6.81 (d, J=16 Hz, 1H); 6.29 (m, 1H); 5.86-5.76 (m, 1H); 5.31 (s, 2H); 5.21 (s, 2H); 5.15-5.07 (m, 3H); 4.59 (s, 2H); 4.16 (m, 1H); 3.86 (s, 2H); 3.89-3.73 (m, 4H); 3.51 (m, 2H); 3.32 (m, 2H); 2.38-2.28 (m, 4H); 1.70 (d, J=6.4 Hz, 3H); 1.68-1.48 (m, 8H); 1.32-1.22 (m, 12H); 0.90 (s, 9H); 0.10 (d, J=8.4 Hz, 6H) ppm. ¹³C NMR (CDCl₃, 400 MHz, 25° C.) δ=166.9, 166.4, 154.1, 153.2, 152.6, 136.0, 134.4, 134.0, 133.7, 125.6, 121.0, 120.8, 117.78, 117.7, 102.6, 94.0, 93.8, 73.07, 73.03, 71.2, 64.7, 64.6, 46.4, 42.9, 42.5, 40.1, 32.6, 26.4, 25.8 (×3), 24.6, 19.3, 18.2, 17.57, 17.55, 15.06, −4.23, −4.77 ppm.

Less polar isomer (20 mg, colorless oil) R_{f}=0.48 (50/50 petroleum ether/EtOAc); ¹H NMR (CDCl₃, 400 MHz, 23° C.) (E)-Oxime δ=7.08 (s, 1H); 6.15-6.08 (m, 1H); 5.86-5.76 (m, 2H); 5.61-5.50 (m, 2H); 5.31 (s, 2H); 5.21 (s, 2H); 5.19-5.11 (m, 3H); 4.69 (s, 2H); 4.01-3.88 (m, 2H); 3.79-3.71 (m, 4H); 3.58-3.55 (m, 2H); 3.41-3.39 (m, 2H); 2.49-3.32 (m, 2H); 2.25-2.12 (m, 2H); 1.65-1.53 (m, 10H); 1.34-1.23 (m, 9H); 0.88 (s, 9H); −0.034 (d, J=8.4 Hz, 6H) ppm. ¹³C NMR (CDCl₃, 400 MHz, 23° C.) δ=166.9, 166.4, 154.1, 153.2, 152.6, 136.0, 134.4, 134.0, 133.7, 125.6, 121.0, 120.8, 117.78, 117.7, 102.6, 94.0, 93.8, 73.07, 73.03, 71.2, 64.7, 64.6, 46.4, 42.9, 42.5, 40.1, 32.6, 26.4, 25.8 (×3), 24.6, 19.3, 18.2, 17.57, 17.55, 15.06, −4.23, −4.77 ppm.

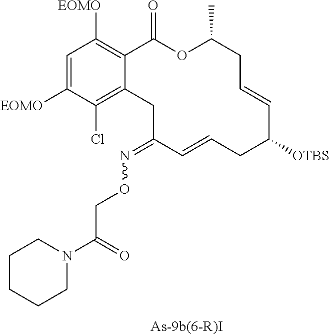

### Synthesis of the Protected Pochoxime As-9b(6-R)I.

A solution of As-9b(6-R) (50.0 mg, 0.066 mmol) in degassed toluene (3.5 mL) under a stream of N₂, was heated to 80° C. To this solution was added Grubbs II catalyst [2×(0.05 mol %, 3.0 mg)] and the mixture was heated for 8 hours. The solution was cooled to room temperature and then DMSO (31.0 μl, 60 equiv respects to the catalyst) was added. The resulting solution was stirred for 24 hours at room temperature, and then filtered over a pad of celite to remove the catalyst. After evaporation of the solvent under reduced pressure, the residue was purified by flash chromatography (silica gel, petroleum ether/EtOAc, 50/50), to afford the desired compound As-9b(6-R)I (colorless oil) in 63% yield (31 mg) as a mixture of E/Z isomers in the ratio of 1/1. Rf=0.36 (50/50 petroleum ether/EtOAc); ¹H NMR (CDCl₃, 400 MHz, 23° C.) δ=7.05 (s, 1H); 7.01 (s, 1H); 6.59 (d, J=16.4 Hz; 1H); 6.07-5.97 (m, 3H); 5.31-5.28 (m, 4H); 5.24-5.21 (m, 4H); 5.12-5.08 (m, 2H); 4.83-4.73 (m, 4H); 4.28-4.23 (m, 2H); 3.81-3.72 (m, 8H); 3.60-3.56 (m, 4H); 3.51-3.46 (m, 4H); 2.41-2.07 (m, 8H); 1.66-1.57 (m, 12H); 1.46 (d, J=6.4 Hz, 3H); 1.38 (d, J=6.4 Hz, 3H); 1.30-1.21 (m, 18H); 0.92 (s, 18H); 0.04 (s, 12H) ppm.

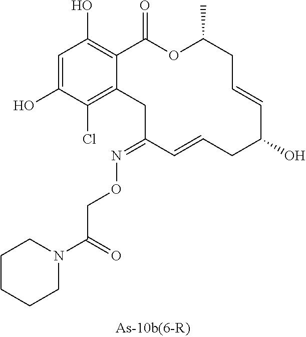 [SMILES: C[C@@H]1C/C=C/[C@H](O)C/C=C/C(=N\OCC(=O)N2CCCCC2)Cc2c(Cl)c(O)cc(O)c2C(=O)O1]

### Synthesis of Pochoxime As-10b(6-R)

To a solution of As-9b(6-R)I (20.0 mg, 0.021 mmol, 1.0 equiv) in methanol (2.0 mL) sulfonic acid resin (111 mg, 0.34 mmol, 10 equiv, 3.0 mmol/g) was added and heated at 40° C. for 16 hours. The reaction mixture was filtered and the resin was rinsed with CH₂Cl₂ (5.0 mL) and MeOH (5.0 mL). After removal of solvents under reduced pressure, the residue was purified by HPLC (20-80% CH3CN in Water gradient in 50 min, flow: 2 mL/min, Discovery® HS C18, 5 μm, 5 cm×10.0 mm) to afford pochoximes As-10a(6-R) (Z-isomer: 2.4 mg; E-isomer: 2.8 mg). ¹H NMR (Acetone-d⁶, 400 MHz, 23° C.) δ=6.58 (s, 1H); 6.08 (m, 1H); 5.59 (m, 1H); 5.41 (d, J=15.6 Hz, 1H); 5.27 (m, 2H); 4.80 (s, 2H); 4.29 (d, J=17.2 Hz, 1H); 4.22 (m, 1H); 4.15 (d, J=17.2 Hz, 1H); 3.54 (m, 4H); 2.56 (m, 1H); 2.32 (m, 1H); 2.18 (m, 2H); 1.66 (m, 4H); 1.53 (m, 2H); 1.40 (d, J=6.4 Hz, 3H) ppm, 3 OH signals are not visible. ¹³C NMR (Acetone-d⁶, 100 MHz, 23° C.) δ=169.21, 167.1, 157.8, 155.1, 138.6, 137.5, 133.1, 126.8, 124.7, 123.6, 115.2, 103.6, 74.0, 72.9, 71.8, 46.8, 43.2, 41.1, 38.3, 35.7, 32.0, 27.3, 26.3, 25.2, 18.8 ppm.

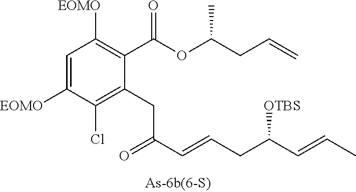

### Synthesis of As-6b(6-S):

A solution of ester As-1b (108 mg, 0.279 mmol, 1.5 equiv) in anhydrous THF (1.5 mL) at −78° C. was treated with freshly prepared LDA (0.409 M, 0.409 mmol, 2.2 equiv) via cannula. After 30 minutes, a solution of Weinreb amide S-As-2 (58 mg, 0.180 mmol, 1.0 equiv) in THF (1.5 mL) at −78° C. was added by syringe. The resulting mixture was then stirred for 25 minutes and the reaction was quenched by addition of sat. NH₄Cl_{aq} solution. Upon warming to 23° C., the reaction mixture was extracted with EtOAc (2×30 mL) and the combined organic layers were washed with brine (30 mL), dried over Na₂SO₄, filtered and concentrated. The residue was purified by flash chromatography (90/20 petroleum ether/EtOAc) to give As-6b(6-S) as a yellow oil in 62% yield (69.4 mg). Rf=0.48 (80/20 petroleum ether/EtOAc); ¹H NMR (CDCl₃, 400 MHz, 23° C.) δ=7.13 (s, 1H); 6.97-6.90 (m, 1H); 6.22 (d, J=16 Hz, 1H); 5.86-5.76 (m, 1H); 5.64-5.57 (m, 1H); 5.47 (dd, J=15.2, 6.8 Hz, 1H); 5.32 (s, 2H); 5.22 (s, 2H); 5.20-5.07 (m, 3H); 4.23-4.18 (m, 1H); 4.07 (s, 2H); 3.80-3.71 (m, 4H); 2.49-2.30 (m, 4H); 1.71 (d, J=6.4 Hz, 3H); 1.30-1.23 (m, 9H); 0.90 (s, 9H); 0.070 (d, J=8.4 Hz, 6H) ppm. ¹³C NMR (CDCl₃, 100 MHz, 23° C.) δ=194.3, 166.5, 154.5, 153.8, 144.4, 133.75, 133.70, 132.4, 131.0, 125.9, 120.4, 117.7, 117.6, 102.8, 93.8, 93.7, 72.5, 71.4, 64.6, 64.4, 42.7, 41.7, 40.1, 25.8 (×3), 19.4, 18.2, 17.5, 15.0, 14.97, −4.2, −4.7 ppm.

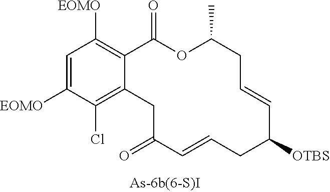

### Synthesis of Compound As-6b(6-S)I:

A solution of As-6b(6-S) (34.0 mg, 0.054 mmol, 1.0 equiv) in toluene (1.0 mL) was degassed for 20 min and heated to 80° C. Grubb's II catalyst (3.0 mg, 24 μmol, 0.05 equiv) was added and stirred at 80° C. for 5 hours. After cooling down to 23° C., the reaction was treated with DMSO (11 μL, 60 equiv. to catalyst) for 24 hours. The mix was passed through silica pad, and washed with petroleum ether/ethyl acetate 50/50 and then 25/75. The combined filtrates were concentrated under reduced pressure and the residue was purified by flash chromatography (90/10 petroleum ether/EtOAc) to afford As-6b(6-S)I in 35% yield (11.2 mg) R_{f}=0.3 (80/20 petroleum ether/EtOAc)¹H (CDCl₃, 400 MHz, 25° C.) δ=7.11 (s, 1H), 6.77-6.69 (m, 1H), 5.87 (d, J=15.6 Hz, 1H); 5.47-5.39 (m, 1H), 5.34 (s, 2H), 5.33-5.31 (m, 1H), 5.26 (s, 2H), 5.18-5.14 (m, 1H), 4.32-4.28 (m, 1H), 4.03 (d, J=17.2 Hz, 1H), 3.86-3.67 (m, 5H), 2.36-2.25 (m, 4H), 1.36 (d, J=6.8 Hz, 3H), 1.30-1.23 (m, 6H), 0.90 (s, 9H), 0.07 (s, 3H), 0.04 (s, 3H) ppm. ¹³C NMR (CDCl₃, 100 MHz, 23° C.) δ=195.6, 166.4, 154.6, 153.6, 143.4, 135.9, 132.7, 130.3, 125.0, 120.5, 117.8, 102.9, 93.9, 93.6, 72.0, 71.8, 64.7, 64.6, 44.5, 40.3, 38.2, 29.6, 25.8 (×3), 18.1, 15.0 (×2), −4.6, −4.9 ppm.

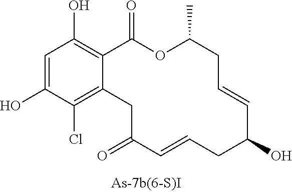 [SMILES: C[C@@H]1C/C=C/[C@@H](O)C/C=C/C(=O)Cc2c(Cl)c(O)cc(O)c2C(=O)O1]

### Synthesis of As-7b(6-S):

To a solution of As-6b(6-S)I (11.2 mg, 0.0188 mmol, 1.0 equiv) in isopropanol (1.0 mL), sulfonic acid resin (37 mg, 0.1128 mmol, 6.0 equiv, 3 mmol/g) was added and heated at 40° C. for 16 hours. The reaction mixture was filtered and the resin was rinsed with CH₂Cl₂ (5.0 mL) and MeOH (5.0 mL). After removal of solvents under reduced pressure, the residue was purified by prep. TLC (40/60 petroleum ether/EtOAc) to afford As-7b(6-S) in 47% yield (3.0 mg) R_{f}=0.24 (40/60 petroleum ether/EtOAc)¹H (CDCl₃, 400 MHz, 25° C.) δ=6.65-6.57 (m, 1H), 6.39 (s, 1H), 5.73 (d, J=15.2 Hz, 1H), 5.42-5.34 (m, 1H), 5.27-5.21 (m, 1H), 5.12-5.06 (m, 1H), 4.12-4.11 (m, 1H), 4.08 (d, J=18 Hz, 1H), 3.91 (d, J=18 Hz, 1H), 2.49-2.43 (m, 2H), 2.24-2.16 (m, 1H), 2.05-1.91 (m, 1H), 1.18 (d, J=6.4 Hz, 3H) ppm. ¹³C NMR (CDCl₃, 100 MHz, 25° C.) δ=198.7, 170.7, 163.1, 159.7, 145.2, 137.1, 131.7, 126.8, 116.6, 109.1, 104.0, 73.5, 73.2, 46.9, 41.0, 37.7, 30.7, 18.0 ppm.

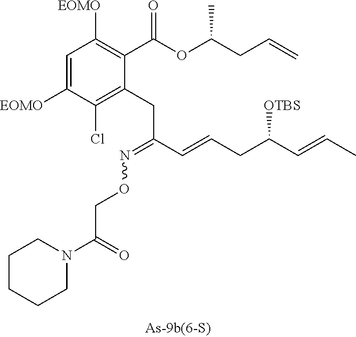

### Synthesis of the Open Chain Oxime As-9b(6-S):

To a stirred solution of As-6b(6-S) (41.5 mg, 0.066 mmol, 1.0 equiv) in pyridine (3.0 mL), at 45° C. under N₂, was added hydroxylamine hydrochloride As-8 (38.8 mg, 0.200 mmol, 3.0 equiv) in two portions over 48 hours. After evaporation of the pyridine, the residue was dissolved in CH₂Cl₂ (30 mL), washed with sat. NH₄Cl_{aq} (10 mL) and dried over anhydrous Na₂SO₄. Filtration and evaporation of the solvents under reduced pressure, followed by flash chromatography (silica gel, gradient from 60/40 to 50/50 petroleum ether/EtOAc) afforded the desired compound As-9b(6-S) (colorless oil) in 53% yield (27 mg) as a mixture of E/Z isomers in the ratio of 1/1. Rf=0.57 (40/60 petroleum ether/EtOAc) ¹H NMR (CDCl₃, 400 MHz, 23° C.) δ=7.13-7.07 (m, 2H); 6.81 (d, J=16.4 Hz, 1H); 6.28-6.21 (m, 1H); 6.15-6.07 (m; 1H); 5.87-5.72 (m, 3H); 5.62-5.37 (m, 4H); 5.31 (s, 4H); 5.21 (s, 4H); 5.19-5.10 (m, 6H); 4.75 (s, 2H); 4.59 (s, 2H); 4.33-4.31 (m; 2H); 4.15-4.11 (m, 1H); 3.99-3.86 (m, 4H); 3.81-3.71 (m, 8H); 3.56-3.51 (m, 4H); 3.41-3.39 (m, 2H); 3.33-3.31 (m, 2H); 2.44-2.11 (m, 8H); 1.70-1.48 (m, 20H); 1.32-1.19 (m, 18H); 0.91 (s, 9H); 0.87 (s, 9H); 0.1-0.001 (m, 12H) ppm. ¹³C NMR (CDCl₃, 100 MHz, 23° C.) (E/Z isomers mixture) δ=166.9, 166.7, 166.4, 166.2, 155.1, 154.6, 154.3, 154.2, 154.1, 153.9, 153.3, 153.2, 152.6, 135.9, 134.41, 134.38, 133.98, 133.87, 133.7, 133.5, 132.9, 125.6, 125.2, 121.1, 120.85, 120.82, 117.88, 117.78, 117.7, 102.8, 102.6, 102.5, 94.0, 93.99, 93.91, 93.8, 93.74, 93.72, 73.3, 73.1, 73.08, 73.03, 71.6, 71.2, 64.7, 64.6, 64.5, 64.4, 46.4, 46.2, 42.99, 42.94, 42.5, 42.4, 40.15, 40.09, 32.6, 26.5, 26.4, 26.3, 25.9 (×3), 25.8 (×3), 25.57, 25.52, 24.6, 24.5, 19.3, 19.26, 18.2, 17.57, 17.55, 15.1, 15.0, −4.2, −4.4, −4.6, −4.7 ppm.

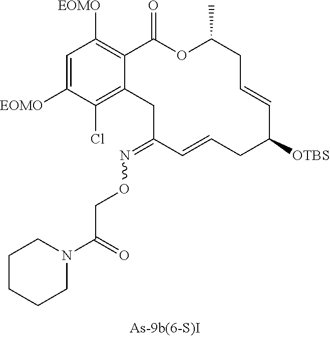

### Synthesis of the Protected Pochoxime As-9b(6-S)I.

A solution of As-9b(6-S) (56.6 mg, 0.074 mmol) in degassed toluene (3.5 mL) under a stream of N₂, was heated to 80° C. To this solution was added Grubbs II catalyst [2×(0.05 mol %, 3.15 mg)] and the mixture was heated for 8 hours. The solution was cooled to room temperature and then DMSO (31.53 μl, 60 equiv respect to the catalyst) was added. The resulting solution was stirred for 24 hours at room temperature, and then filtered over a pad of celite to remove the catalyst. After evaporation of the solvent under reduced pressure, the residue was purified by flash chromatography (silica gel, petroleum ether/EtOAc, 50/50), to afford the desired compound As-9b(6-S)I (colorless oil) in 67% yield (36 mg) as a mixture of E/Z isomers in the ratio of 1/1. Rf=0.36 (50/50 petroleum ether/EtOAc); ¹H NMR (CDCl₃, 400 MHz, 23° C.) δ=7.05 (s, 1H); 7.01 (s, 1H); 6.59 (d, J=16.4 Hz; 1H); 6.07-5.97 (m, 3H); 5.31-5.28 (m, 4H); 5.24-5.21 (m, 4H); 5.12-5.08 (m, 2H); 4.83-4.73 (m, 4H); 4.28-4.23 (m, 2H); 3.81-3.72 (m, 8H); 3.60-3.56 (m, 4H); 3.51-3.46 (m, 4H); 2.41-2.07 (m, 8H); 1.66-1.57 (m, 12H); 1.46 (d, J=6.4 Hz, 3H); 1.38 (d, J=6.4 Hz, 3H); 1.30-1.21 (m, 18H); 0.92 (s, 18H); 0.04 (s, 12H) ppm.

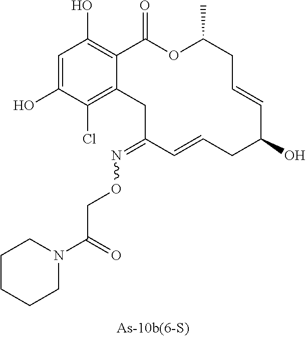 [SMILES: C[C@@H]1C/C=C/[C@@H](O)C/C=C/C(=NOCC(=O)N2CCCCC2)Cc2c(Cl)c(O)cc(O)c2C(=O)O1]

### Synthesis of As-10b(6-S):

To a solution of As-9b(6-S)I (36 mg, 0.048 mmol, 1.0 equiv) in isopropanol (2.0 mL), sulfonic acid resin (95 mg, 0.293 mmol, 6.0 equiv) was added and heated at 40° C. for 16 hours. The reaction mixture was filtered and the resin was rinsed with CH₂Cl₂ (5.0 mL) and MeOH (5.0 mL). After removal of solvents under reduced pressure, the residue was purified by flash chromatography (silica gel, 50/50 petroleum ether/EtOAc) to afford the desired compound As-10b(6-S) (colorless oil) in 77% yield (19 mg) as a mixture of E/Z isomers in the ratio of 1/1. R_{f}=0.69 (30/70 petroleum ether/EtOAc). The mixture was subsequently purified by HPLC (20-80% CH3CN in Water gradient in 50 min, flow: 2 mL/min, Discovery® HS C18, 5 μm, 5 cm×10.0 mm) to afford pure As-10b(6-S): ¹H (Acetone-d⁶, 400 MHz, 25° C.) δ=6.45 (s, 1H), 5.95-5.88 (m, 1H), 5.38-5.31 (m, 1H), 5.18-5.11 (m, 2H), 5.05 (dd, J=15.2 Hz, 8 Hz, 1H), 4.65 (s, 2H), 4.15 (d, J=17.2 Hz, 1H), 4.02 (m, 1H), 4.02 (d, J=17.2 Hz, 1H), 3.42 (m, 4H), 2.47-2.41 (m, 1H), 2.31-2.27 (m, 1H), 2.19-2.16 (m, 1H), 1.79 (m, 1H), 1.51-1.38 (m, 6H), 1.24 (d, J=6.4 Hz, 3H) ppm. ¹³C NMR (Acetone-d⁶, 100 MHz, 25° C.) δ=169.20, 167.0, 158.2, 154.2, 139.0, 138.0, 137.5, 125.5, 123.2, 116.0, 109.3, 103.3, 73.5, 73.0, 70.7, 46.8, 43.1, 40.9, 36.8, 35.7, 31.0, 27.1, 26.3, 25.2, 18.8 ppm.

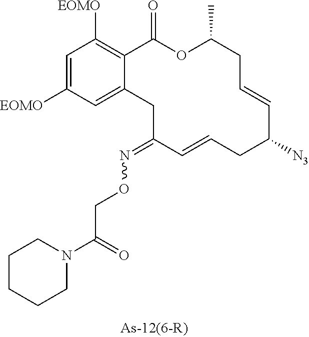

### Synthesis of Azide As-12(6-R):

A solution of As-9a(6-S)I (200 mg, 0.28 mmol) in THF (3.0 mL) was treated with a solution of TBAF in THF (0.42 mL, 1M in THF, 1.5 equiv) at 0° C. The reaction was allowed to reach room temperature and was stirred for 3 hours. Then the mixture was extracted from sat. NH₄Cl_{aq} solution with EtOAc (3×10 mL), washed with brine (15 mL) dried over anhydrous Na₂SO₄, and concentrated. Purification by flash chromatography column (EtOAc) afforded the corresponding TBS deprotected alcohol As-11a(6-S) in 72% yield (120 mg). The alcohol As-11a(6-S) (110 mg, 0.187 mmol, 1.0 equiv) was dissolved in CH₂Cl₂ (5.0 mL) and the solution was cooled to 0° C. Triethylamine (130 μL, 0.93 mmol, 5.0 equiv) and methane sulphonyl chloride (58 μL, and 0.748 mmol, 4.0 equiv) were added slowly at 0° C. The reaction mixture was stirred for 7 hours at 23° C. and then quenched with sat. aqueous NaHCO₃ and washed with brine, dried over Na₂SO₄, filtered and the solvent was concentrated under reduced pressure to leave a yellow residue. The crude mesylate was dissolved in DMF (5.0 mL) NaN₃ (204 mg, 3.74 mmol, 20 equiv) was added and the mixture was stirred at 23° C. for 24 hours. Then, the reaction mixture was filtered and concentrated under reduced pressure. The residue was purified by flash chromatography (SiO₂, gradient from 70/30 to 50/50 hexane/EtOAc), to afford azide As-12(6-R) in 74% over two steps (85 mg) as a colourless oil. R_{f}=0.3 (50/50 EtOAc/Hexane); ¹H NMR (CDCl₃, 400 MHz) δ=6.87 (d, J=16.4 Hz, 1H), 6.74-6.72 (m, 2H), 6.65 (s, 1H), 6.59 (s, 1H), 6.14 (d, J=16.0 Hz, 1H), 5.78-5.60 (m, 4H), 5.40-5.34 (m, 2H), 5.24 (s, 8H), 5.20-5.14 (m, 2H), 4.83 (s, 4H), 4.53 (d, J=14.8 Hz, 1H), 4.20-4.13 (m, 1H), 3.71-3.68 (m, 10H), 3.61-3.57 (m, 5H), 3.48-3.39 (m, 4H), 3.11 (d, J=14.8 Hz, 1H), 2.52-2.48 (m, 2H), 2.42-2.35 (m, 4H), 2.17-2.14 (m, 2H), 1.68-1.58 (m, 12H), 1.46 (d, J=6.4 Hz, 6H), 1.30-1.20 (m, 12H) ppm.

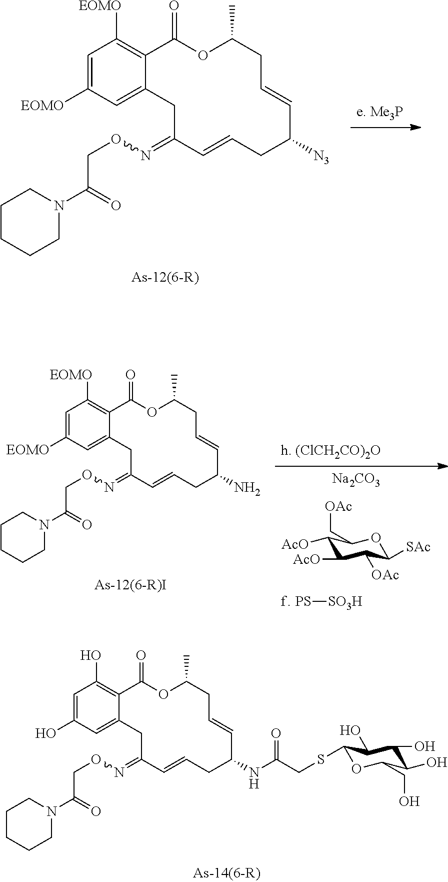

### Synthesis of GlycoPochoxime As-14(6-R).

To the solution of azide As-12(6-R) (80 mg, 0.13 mmol, 1.0 equiv) in a mixture of THF: H₂O (5.5 mL, 5:1) was added Me₃P (0.52 mL, 4.0 equiv, 1M in THF). The reaction mixture was stirred at 23° C. for 5 hours and then concentrated under reduced pressure. The crude amine As-12(6-R)I was dissolved in CH₂Cl₂ (5 mL) and cooled to 0° C., i-Pr₂EtN (0.1 mL, 0.65 mmol, 5.0 equiv) and chloroacetic anhydride (0.11 g, 0.65 mmol, 5.0 equiv) were added sequentially and stirred for 10 min. The reaction mixture was quenched with aqueous NaHCO₃. The organic layer was then washed with brine, dried over Na₂SO₄ and concentrated under reduce pressure. The residue was purified by flash chromatography (SiO₂, 40/60 Hexane/EtOAc) to give the corresponding chloride in 45.3% over two steps (39.1 mg). R_{f}=0.15(1:1 EtOAc/Hexane). LC-MS: m/z [M]⁺ calcd. for C₃₃H₄₆ClN₃O₉: 663.29; found: 662.85. To a solution of tetra-O-acetyl-1-S-acetyl-1-thio-β-D-glucopyranose (72.7 mg, 0.17 mmol, 3.0 equiv) in methanol (2.0 mL), sodium carbonate (93.5 mg, 0.88 mmol, 15.0 equiv) was added and the mixture was stirred at room temperature for 3 hours; then, chloride (39.1 mg, 0.058 mmol, 1.0 equiv) was added and the stirring continued further for 3 hours. The reaction mixture was filtered, neutralized with Dowex 50WX2-100 resin and the solvent was concentrated under reduce pressure. The crude product was dissolved in methanol (3.0 mL) and PS—SO₃H (193 mg, 0.58 mmol, 10.0 equiv, 3.0 mmol/g) was added and the resulting suspension was stirred at 23° C. for 16 hours. The reaction mixture was filtered, and the solvent was concentrated under reduced pressure. The crude product was purified by HPLC (Agilent 1100 series HPLC equipped with DAD and with a Agilent ZORBAX Eclipse XDB-C18 (4.6×300 mm, 5 μm) column (linear gradient from 70% H2O 0.1% TFA 30% MeCN 0.1% TFA to 50% H2O 0.1% TFA 50% MeCN 0.1% TFA in 35 minutes with a flow rate of 2.0 mL/min) to give thio glycosides As-14(6-R) in 52% over two steps (Z-isomer: 9.4 mg; E-isomer: 11.8 mg). ¹H NMR (Z-isomer, CD₃OD, 400 MHz) δ=6.69 (d, J=16 Hz, 1H), 6.16 (d, J=2.0 Hz, 1H), 6.07 (d, J=2.0 Hz, 1H), 5.95-5.87 (m, 1H), 5.54-5.46 (m, 1H), 5.29 (dd, J=15.2, 8.8 Hz, 1H), 5.17-5.13 (m, 1H), 4.70 (d, J=4.0 Hz, 2H), 4.28 (d, J=10 Hz, 1H), 4.13-4.07 (m, 1H), 3.75 (d, J=12 Hz, 1H), 3.54-3.45 (m, 5H), 3.38 (t, J=5.2 Hz, 2H), 3.18-3.17 (m, 4H), 3.14-3.08 (m, 2H), 2.43-2.22 (m, 3H), 2.13-2.05 (m, 1H), 1.59-1.48 (m, 6H), 1.30 (d, J=6.0 Hz, 3H) ppm (all the OH and NH are not visible); ¹³C NMR (Z-isomer, CD₃OD, 100 MHz) δ 170.3, 169.2, 168.3, 160.3, 158.8, 155.2, 139.3, 137.7, 131.9, 129.3, 120.03, 106.8, 100.8, 85.1, 80.8, 78.1, 72.9, 71.3, 70.9, 69.7, 61.5, 52.6, 45.8, 42.8, 39.0, 38.7, 37.4, 34.5, 32.5, 26.0, 25.3, 23.9, 18.8 ppm.

¹H NMR (E-isomer, CD₃OD, 400 MHz) δ=6.21 (s, 1H), 6.19 (d, J=1.6 Hz, 1H), 6.05 (d, J=16 Hz, 1H), 5.97-5.89 (m, 1H), 5.67-5.60 (m, 1H), 5.41 (dd, J=15.2, 8.4 Hz, 1H), 5.29-5.23 (m, 1H), 4.42 (s, 2H), 4.39 (d, J=5.6 Hz, 1H), 4.20-4.15 (m, 1H), 3.88 (d, J=11.6 Hz, 1H), 3.66-3.62 (m, 2H), 3.59-3.50 (m, 4H), 3.47-3.44 (m, 4H), 3.31 (s, 2H), 2.55-2.51 (m, 1H), 2.44-2.36 (m, 2H), 2.22-2.15 (m, 1H), 1.72-1.62 (m, 6H), 1.45 (d, J=6.0 Hz, 3H) ppm, (all the OH and NH are not visible); ¹³C NMR (Z-isomer, CD₃OD, 100 MHz) δ 170.2, 169.1, 168.0, 160.2, 157.9, 137.9, 134.0, 131.8, 129.5, 126.8, 110.9, 106.8, 100.6, 85.0, 80.8, 78.0, 72.9, 71.5, 71.0, 70.0, 61.5, 52.8, 48.5, 45.9, 42.8, 39.1, 36.9, 32.5, 28.2, 26.1, 25.3, 23.9, 19.0 ppm.

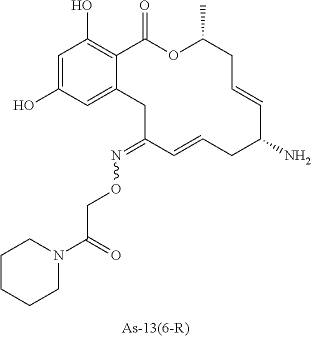 [SMILES: C[C@@H]1C/C=C/[C@H](N)C/C=C/C(=NOCC(=O)N2CCCCC2)Cc2cc(O)cc(O)c2C(=O)O1]

### Synthesis of Amine As-13(6-R).

The amine As-12(6-R)I (10 mg, 0.017 mmol) was dissolved in TFA/m-cresol 2:1 (100 μL/50 μL) and stirred at room temperature for 5 min. The reaction mixture was then diluted with 10 mL H₂O, lyophilized to give amine As-13(6-R) in a 62% yield (5.0 mg) and purified by HPLC (Agilent 1100 series HPLC equipped with DAD and with a Agilent ZORBAX Eclipse XDB-C18 (4.6×300 mm, 5 μm) column (linear gradient from 82% H₂O 0.1% TFA 18% MeCN 0.1% TFA to 64% H₂O 0.1% TFA 36% MeCN 0.1% TFA in 35 minutes with a flow rate of 2.0 mL/min). ¹H NMR (CD₃OD, 400 MHz) δ=6.91 (d, J=16 Hz, 1H), 6.27 (d, J=1.6 Hz, 1H), 6.19 (s, 3H), 6.12 (d, J=15.6 Hz, 1H), 5.91-5.78 (m, 4H), 5.46-5.40 (m, 2H), 5.29-5.23 (m, 2H), 4.42 (d, J=15.2 Hz, 1H), 3.68-3.55 (m, 10H), 3.54-3.49 (m, 7H), 2.69-2.47 (m, 4H), 2.34-2.28 (m, 4H), 1.72-1.71 (m, 6H), 1.63-1.62 (m, 6H), 1.47 (d, J=6.0 Hz, 6H) ppm.

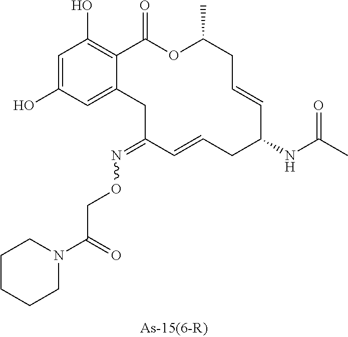 [SMILES: CC(=O)N[C@H]1/C=C/C[C@@H](C)OC(=O)c2c(O)cc(O)cc2CC(=NOCC(=O)N2CCCCC2)/C=C/C1]

### Preparation of Acetamide As-15(6-R).

The amine As-12(6-R)I (20 mg, 0.034 mmol) was dissolved in CH₂Cl₂ (1.0 mL) and cooled to 0° C., i-Pr₂EtN (3.8 μL, 0.17 mmol, 5.0 equiv) and acetic anhydride (17.2 μL, 0.17 mmol, 5.0 equiv) were added sequentially and the mixture was stirred for 10 min. The reaction was then quenched with aqueous NaHCO₃ and the organic layer was washed with brine, dried over Na₂SO₄ and evaporated under reduce pressure. The crude acetyl derivative was dissolved in methanol (3.0 mL) and PS—SO₃H (56 mg, 0.17 mmol, 10.0 equiv, 3.0 mmol/g) was added and stirred at 23° C. for 16 hours. The reaction mixture was filtered, and evaporated under vacuo. The crude product was purified by HPLC (Agilent 1100 series HPLC equipped with DAD and with a Agilent ZORBAX Eclipse XDB-C18 (4.6×300 mm, 5 μm) column (linear gradient from 70% H₂O 0.1% TFA 30% MeCN 0.1% TFA to 50% H₂O 0.1% TFA 50% MeCN 0.1% TFA in 35 minutes with a flow rate of 2.0 mL/min). to give acetamide As-15(6-R)(64%—over 2 steps: Z-isomer: 5.4 mg; E-isomer: 5.8 mg). ¹H NMR (Z-isomer, CDCl₃, 400 MHz) δ=6.67 (d, J=16.4 Hz, 1H), 6.53 (d, J=2.0 Hz, 1H), 6.29 (d, J=2.0 Hz, 1H), 6.08-5.95 (m, 1H), 5.54-5.47 (m, 2H), 5.40-5.35 (m, 1H), 4.62-4.59 (m, 2H), 4.34 (d, J=15.6 Hz, 1H), 3.82 (d, J=15.2 Hz, 1H), 3.55-3.54 (m, 2H), 3.37 (s, 2H), 2.71-2.66 (m, 2H), 2.49-2.45 (m, 2H), 2.33-2.25 (m, 2H), 1.93 (s, 3H), 1.66-1.58 (m, 6H), 1.43 (d, J=6.4 Hz, 3H) ppm two OH signals are not visible.

¹H NMR (E-isomer, CDCl₃, 400 MHz) δ=6.45 (s, 1H), 6.30 (d, J=1.6 Hz, 1H), 5.95 (s, 1H), 5.55-5.50 (m, 3H), 5.41-5.35 (m, 1H), 4.91-4.79 (m, 2H), 4.62-4.56 (m, 1H), 4.37 (d, J=4.4 Hz, 1H), 3.56 (s, 2H), 3.37 (s, 2H), 2.73-2.65 (m, 2H), 2.46-2.21 (m, 4H), 1.99 (s, 3H), 1.66-1.59 (m, 6H), 1.44 (d, J=6.4 Hz, 3H) ppm two OH signals are not visible.

### Methods for Molecular Dynamics

The conformational profile of the pochoxime derivatives were analyzed using the approach known to one skilled in the art. Each molecule was simulated by molecular dynamics with the Merck Molecular Force Field (MMFF94) in the CHARMM program, version c31b1. A dielectric constant of 80 was used to simulate the effect of solvent in a simple way. The simulations were carried out at 1000 K during 10 ns and 2000 frames were extracted from the trajectory at 10 ps intervals. The high temperature was used to ensure that conformational energy barriers were crossed. Each frame was minimized by 2000 steps of the steepest descent (SD) algorithm in CHARM, and the MMFF energy was calculated. The resulting 2000 conformations were clustered to determine the main conformations. Starting from the lowest energy conformation as representative of the first cluster, all conformations having a root-mean-square deviation (RMSD) lower than 1 Å were grouped into that cluster. The lowest energy conformer of the remaining conformations was taken as the starting point for the second cluster, and the process was repeated until all compounds had been clustered.

Docking of pochoxime derivatives in the HSP90 binding site were performed using three different approaches: Autodock 4, AutodockVina and a developmental algorithm called “Attracting cavities” that will be implemented in EADock. In brief, in the latter, an extended conformation of the ligand is minimized in the cavities of the protein, starting from different positions and orientations, in an approach similar to that of MCSS. Minimized poses are finally clustered and ranked according to the scoring function of EADock. A detailed description of this algorithm will be the subject of a future communication.

All docking runs were performed using the HSP90 structure (PDB ID 3INW), after removing the ligand. Docking were done in a 30 Å³ cubic box centered on the center of the ligand originally present in the 3INW PDB file. Two water molecules present in the 3INW structure were retained in the docking calculations, since they contribute to the existence of hydrogen bond bridges between the phenol functions present in all ligands and residues Asp93 and Ser52 of HSP90.

Autodock calculations were performed using 100 GA runs, each involving a maximum of 12,500,000 energy calculations. Vina calculations were performed using an exhaustively value of 100. Default values were used for all other parameters.

The 6 new pochoximes, as well as the two ligands present in PDB ID 3INW and 3INX, and radicicol were all docked using two separate series of docking runs. In the first series, docking software were fed with the bioactive conformation for radicicol and the 3INW and 3INX ligands, and with the P-shape conformer for the new pochoximes. In a second series, all docking programs were fed with the bioactive L-shape conformer for all the ligands.

### MMFF94:

Halgren, T. A. J. Comput. Chem. 1996, 17, 616-641.
Halgren, T. A.; Nachbar, R. B. J. Comput. Chem. 1996, 17, 587-615
Halgren, T. A. J. Comput. Chem. 1996, 17, 553-586.
Halgren, T. A. J. Comput. Chem. 1996, 17, 520-552.
Halgren, T. A. J. Comput. Chem. 1996, 17, 490-519.

### CHARMM:

Brooks, B. R.; Bruccoleri, R. E.; Olafson, B. D.; States, D. J.; Swaminathan, S.; Karplus, M. J. Comput. Chem. 1983, 4, 187-217

### Autodock 4:

Morris G M, Huey R, Lindstrom W, Sanner M F, Belew R K, Goodsell D S, Olson A J. J Comput Chem. 2009 December; 30(16):2785-91.

### AutodockVina:

Trott O, Olson A J. J Comput Chem. 2010 Jan. 30; 31(2):455-61.

### EADock:

Grosdidier A, Zoete V, Michielin O. J Comput Chem. 2009 October; 30(13):2021-30.

### HSP90 Affinity Bioassay

### Fluorescence Polarization Hsp90α Competition Assay

Fluoresceine-GA was purchased from InvivoGen and dissolved in DMSO to form a 1 mM solution. HSP90α was purchased from Stressgen (SPP-776F). The assay buffer contained 20 mM HEPES (K), pH 7.3, 50 mM KCl, 5 mM MgCl₂, 20 mM Na₂MoO₄, 0.01% Tergitol® solution Type NP-40, 70% in H2O (Sigma-Aldrich, NP40S). Before each use, 0.1 mg/mL bovine gamma globulin (BGG; Calbiochem, 345876) and 2 mM DTT (Fluka, 43817) were freshly added. Fluorescence polarizations measurements were performed on a Molecular Devices instrument, reading black 96-well plates (Corning, 3650) from the top of the wells. Measurements were made with excitation at 485 nm and emission at 538 nm with a cutoff of 530 nm. The polarization values were calculated using the equation mP=1000×[(I_{S}−I_{SB})−(I_{P}−I_{PB})]/[(I_{S}−I_{SB})+(I_{P}−I_{PB})], where I_{S} is the parallel emission intensity, I_{P} is the perpendicular emission intensity and I_{SB} and I_{PB} are the values for the background.

Stocks solutions of the compounds were made in DMSO at concentrations of 10 mM. The drugs were serially diluted over a threefold dilution in assay buffer starting from 30 μM. GA-FITC and Hsp90 were added at 5 and 25 nM concentrations respectively. Total reaction mixture was of 100 μL. The plates were shaked at 4° C. for 8 h in the dark and then the FP values were recorded. A window of 100 mP was observed between wells containing protein and tracer and wells containing tracer only. The measured FP values (mP) were plotted against the competitor concentration. EC50 values were determined as the competitor concentration where 50% of GA was displaced.

### Cellular Assay Results for the New Amino Pochoxime Compounds—Example

The compounds were dissolved in 100% DMSO to make 10 mM stock solutions. HER2+ BT474 breast carcinoma cells were cultured in DMEM/F12 media, supplemented with 10% FBS. Log-phase growing BT474 cells were seeded in 96 well plates at 1.5×10E4 per well. At this cell density, BT474 is expected to reach confluency about 70-80% in 3 days. Different dilutions of compounds or vehicle in 200 μl (concentration range 0.004-10 μM) were added to the cells and incubated for 72 hours. Upon completion of the incubation, media were gently removed by suction and 100 μl of ATPlite solution (Perkin Elmer) was added in each well. Viable cells were measured by detecting luminescence generated from reaction of ATPlite solution and ATP in the cells using a 96-well microplate luminescence reader. Relative luminescence light unit is correlated to the amount of ATP in viable cells. The assays were performed in duplicates. IC₅₀ was calculated using XLfit. The IC₅₀ shown in Table 4 below is the average IC₅₀ from three independent experiments.

TABLE 4
IC₅₀ of the Present Compounds

| Com- | Proliferation assay in | |
|---|---|---|
| pound | BT474 (IC₅₀ μM) | Chemical Structure |
| 5Z | >10 | 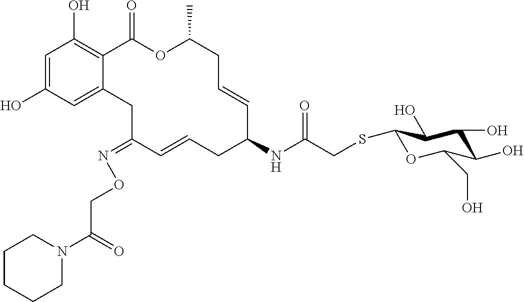 |
| 5E | 7.84 | 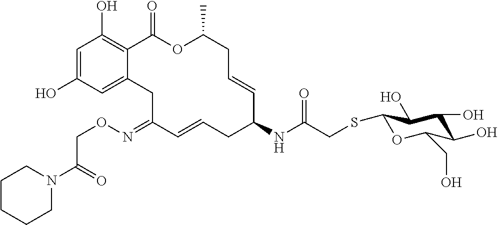 |
| 11Z | >10 | 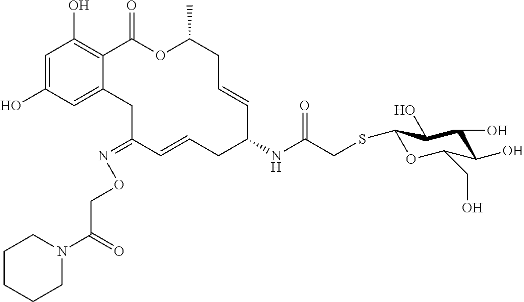 |
| 11E | 4.76 | 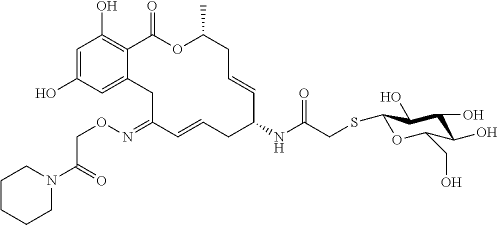 |
| 12Z | 0.038 | 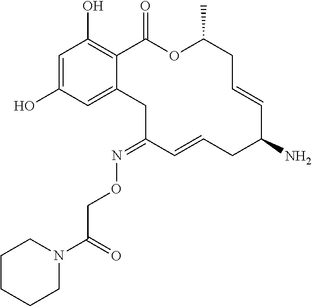 [SMILES: C[C@@H]1C/C=C/[C@@H](N)C/C=C/C(=N\OCC(=O)N2CCCCC2)Cc2cc(O)cc(O)c2C(=O)O1] |
| 12E | 0.030 | 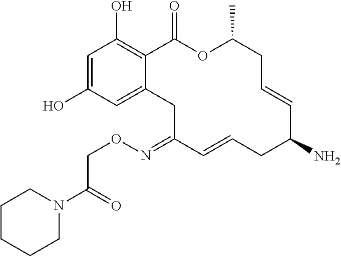 [SMILES: C[C@@H]1C/C=C/[C@@H](N)C/C=C/C(=N/OCC(=O)N2CCCCC2)Cc2cc(O)cc(O)c2C(=O)O1] |
| 13 | 0.097 | 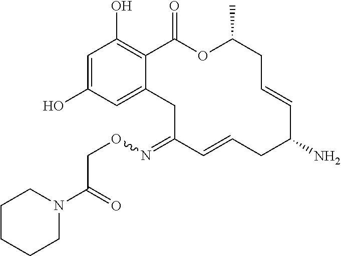 [SMILES: C[C@@H]1C/C=C/[C@H](N)C/C=C/C(=NOCC(=O)N2CCCCC2)Cc2cc(O)cc(O)c2C(=O)O1] |
| 14Z | 0.270 | 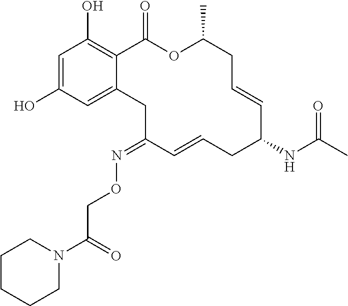 [SMILES: CC(=O)N[C@H]1/C=C/C[C@@H](C)OC(=O)c2c(O)cc(O)cc2CC(=N/OCC(=O)N2CCCCC2)/C=C/C1] |
| 14E | 0.223 | 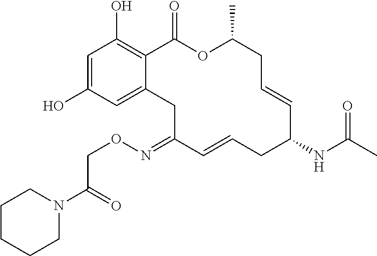 [SMILES: CC(=O)N[C@H]1/C=C/C[C@@H](C)OC(=O)c2c(O)cc(O)cc2CC(=N\OCC(=O)N2CCCCC2)/C=C/C1] |
| As-10a (6-S)-Z | 0.026 | 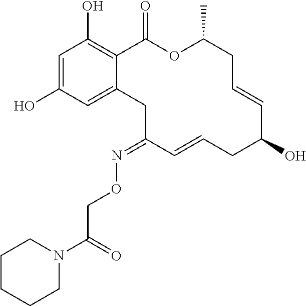 [SMILES: C[C@@H]1C/C=C/[C@@H](O)C/C=C/C(=N\OCC(=O)N2CCCCC2)Cc2cc(O)cc(O)c2C(=O)O1] |
| As-10a (6-S)-E | 0.058 | 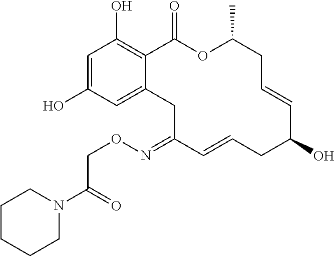 [SMILES: C[C@@H]1C/C=C/[C@@H](O)C/C=C/C(=N/OCC(=O)N2CCCCC2)Cc2cc(O)cc(O)c2C(=O)O1] |
| As-10b (6-S)-Z | 0.824 | 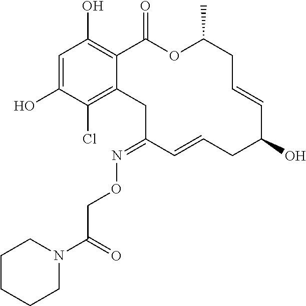 [SMILES: C[C@@H]1C/C=C/[C@@H](O)C/C=C/C(=N\OCC(=O)N2CCCCC2)Cc2c(Cl)c(O)cc(O)c2C(=O)O1] |
| As-10b (6-S)-E | 0.027 | 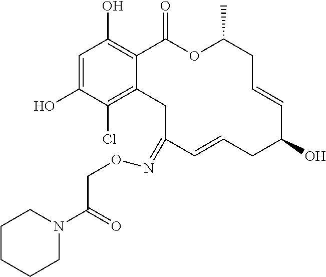 [SMILES: C[C@@H]1C/C=C/[C@@H](O)C/C=C/C(=N/OCC(=O)N2CCCCC2)Cc2c(Cl)c(O)cc(O)c2C(=O)O1] |

Additional proliferation assays were performed for certain exemplified compounds using the methods similar to the one described above in multiple cancer cell lines. IC₅₀ values are shown in Table 5 below.

TABLE 5
Additional IC₅₀ of the Present Compounds

| | | | | | | As- | | |
|---|---|---|---|---|---|---|---|---|
| Cell Lines | | A1B5C1D1-1 | A1B5C1D1-4 | As-13(6S)-Z | As-13(6S)-E | 10(a)(6S)-Z | As-10(a)(6S)-E | As-10(b)(6S)-E |
| HCC1569 | Breast (Her2+; Her2i res) | 0.160 | 0.087 | 0.175 | 0.140 | 0.1 | 0.377 | 0.187 |
| HCC827 | Lung (EGFR mut) | 0.059 | 0.042 | 0.105 | 0.079 | — | — | — |
| H1975 | Lung (EGFR mut) | 0.053 | 0.041 | 0.051 | 0.037 | 0.035 | 0.073 | 0.033 |
| NCI-H2228 | Lung (EML4-ALK+) | 0.164 | 0.046 | 0.139 | 0.087 | 0.077 | 0.241 | 0.281 |
| H23 | Lung | 0.033 | 0.027 | — | 0.041 | 0.028 | 0.088 | 0.036 |
| A549 | Lung | 0.093 | 0.023 | — | — | 0.022 | 0.09 | 0.126 |
| H460 | Lung | 0.029 | 0.016 | — | 0.033 | 0.015 | 0.045 | — |
| N87 | Gastric (Her2+) | 0.041 | 0.047 | 0.012 | 0.007 | 0.011 | 0.073 | 0.013 |
| SKOV-3 | Ovarian (Her2+) | 0.042 | 0.031 | — | 0.033 | 0.025 | 0.067 | 0.045 |
| A498 | Renal (VHL mut) | 0.04 | 0.024 | 0.166 | 0.18 | 0.024 | 0.099 | 0.037 |
| RPMI8226 | Multiple myeloma | 0.046 | 0.04 | 0.072 | 0.046 | — | — | — |
| PC3 | Prostate | 0.062 | 0.037 | 0.082 | 0.044 | — | — | — |
| HCT116 | Colon | 0.084 | 0.042 | 0.101 | 0.076 | — | — | — |
| HT29 | Colon | 1.23 | 1.3 | 0.092 | 0.033 | — | — | — |

### REFERENCES

[1] P. Workman, F. Burrows, L. Neckers, N. Rosen, Ann. N.Y. Acad. Sci. 2007, 1113, 202-216.
[2] L. Neckers, K. Neckers, Expert. Opin. Emerg. Drugs 2005, 10, 137-149.
[3] T. Taldone, W. Sun, G. Chiosis, Bioorg. Med. Chem. Lett. 2009, 17, 2225-2235.
[4] L. Whitesell, S. L. Lindquist, Nat. Rev. Cancer 2005, 5, 761-772.
[5] T. Taldone, A. Gozman, R. Maharaj, G. Chiosis, Curr. Opin. Pharmacol. 2008, 8, 370-374.
[6] A. Sittler, R. Lurz, G. Lueder, J. Priller, H. Lehrach, M. K. Hayer-Hartl, F. U. Hartl, E. E. Wanker, Hum. Mol. Genet. 2001, 10, 1307-1315.
[7] M. Waza, H. Adachi, M. Katsuno, M. Minamiyama, C. Sang, F. Tanaka, A. Inukai, M. Doyu, G. Sobue, Nat. Med. 2005, 11, 1088-1095.
[8] W. Luo, F. Dou, A. Rodina, S. Chip, J. Kim, Q. Zhao, K. Moulick, J. Aguirre, N. Wu, P. Greengard, G. Chiosis, Proc. Natl. Acad. Sci. USA 2007, 104, 9511-9516.
[9] P. K. Auluck, N. M. Bonini, Nat. Med. 2002, 8, 1185-1186.
[10] W. Luo, A. Rodina, G. Chiosis, BMC Neurosci. 2008, 9 Suppl 2, S7.
[11] R. Geller, M. Vignuzzi, R. Andino, J. Frydman, Genes Dev. 2007, 21, 195-205.
[12] J. W. Rice, J. M. Veal, R. P. Fadden, A. F. Barabasz, J. M. Partridge, T. E. Barta, L. G. Dubois, K. H. Huang, S. R. Mabbett, M. A. Silinski, P. M. Steed, S. E. Hall, Arthritis Rheum. 2008, 58, 3765-3775.
[13] S. V. Sharma, T. Agatsuma, H. Nakano, Oncogene 1998, 16, 2639-2645.
[14] T. W. Schulte, S. Akinaga, S. Soga, W. Sullivan, B. Stensgard, D. Toft, L. M. Neckers, Cell Stress Chaperones 1998, 3, 100-108.
[15] L. Neckers, T. W. Schulte, E. Mimnaugh, Invest. New Drugs 1999, 17, 361-373.
[16] G. Chiosis, Y. Kang, W. Sun, Expert Opin. Drug Discovery 2008, 3, 99-114.
[17] K. Lundgren, H. Zhang, J. Brekken, N. Huser, R. E. Powell, N. Timple, D. J. Busch, L. Neely, J. L. Sensintaffar, Y. C. Yang, A. McKenzie, J. Friedman, R. Scannevin, A. Kamal, K. Hong, S. R. Kasibhatla, M. F. Boehm, F. J. Burrows, Mol. Cancer Ther. 2009, 8, 921-929.
[18] S. Y. Sharp, C. Prodromou, K. Boxall, M. V. Powers, J. L. Holmes, G. Box, T. P. Matthews, K.-M. J. Cheung, A. Kalusa, K. James, A. Hayes, A. Hardcastle, B. Dymock, P. A. Brough, X. Barril, J. E. Cansfield, L. Wright, A. Surgenor, N. Foloppe, R. E. Hubbard, W. Aherne, L. Pearl, K. Jones, E. McDonald, F. Raynaud, S. Eccles, M. Drysdale, P. Workman, Mol. Cancer Ther. 2007, 6, 1198-1211.
[19] E. McDonald, K. Jones, P. A. Brough, M. J. Drysdale, P. Workman, Curr. Top. Med. Chem. 2006, 6, 1193-1203.
[20] S. A. Eccles, A. Massey, F. I. Raynaud, S. Y. Sharp, G. Box, M. Valenti, L. Patterson, A. de Haven Brandon, S. Gowan, F. Boxall, W. Aherne, M. Rowlands, A. Hayes, V. Martins, F. Urban, K. Boxall, C. Prodromou, L. Pearl, K. James, T. P. Matthews, K.-M. Cheung, A. Kalusa, K. Jones, E. McDonald, X. Barril, P. A. Brough, J. E. Cansfield, B. Dymock, M. J. Drysdale, H. Finch, R. Howes, R. E. Hubbard, A. Surgenor, P. Webb, M. Wood, L. Wright, P. Workman, Cancer Res. 2008, 68, 2850-2860.
[21] S. Chandarlapaty, A. Sawai, Q. Ye, A. Scott, M. Silinski, K. Huang, P. Fadden, J. Partdrige, S. Hall, P. Steed, L. Norton, N. Rosen, D. B. Solit, Clin. Cancer Res. 2008, 14, 240-248.
[22] K. H. Huang, J. M. Veal, R. P. Fadden, J. W. Rice, J. Eaves, J. P. Strachan, A. F. Barabasz, B. E. Foley, T. E. Barta, W. Ma, M. A. Silinski, M. Hu, J. M. Partridge, A. Scott, L. G. DuBois, T. Freed, P. M. Steed, A. J. Ommen, E. D. Smith, P. F. Hughes, A. R. Woodward, G. J. Hanson, W. S. McCall, C. J. Markworth, L. Hinkley, M. Jenks, L. Geng, M. Lewis, J. Otto, B. Pronk, K. Verleysen, S. E. Hall, J. Med. Chem. 2009, 52, 4288-4305.
[23] G. Chiosis, E. Caldas Lopes, D. Solit, Curr Opin Investig Drugs 2006, 7, 534-541.
[24] S. Modi, A. T. Stopeck, M. S. Gordon, D. Mendelson, D. B. Solit, R. Bagatell, W. Ma, J. Wheler, N. Rosen, L. Norton, G. F. Cropp, R. G. Johnson, A. L. Hannah, C. A. Hudis, J. Clin. Oncol. 2007, 25, 5410-5417.
[25] J. R. Sydor, E. Normant, C. S. Pien, J. R. Porter, J. Ge, L. Grenier, R. H. Pak, J. A. Ali, M. S. Dembski, J. Hudak, J. Patterson, C. Penders, M. Pink, M. A. Read, J. Sang, C. Woodward, Y. Zhang, D. S. Grayzel, J. Wright, J. A. Barrett, V. J. Palombella, J. Adams, J. K. Tong, Proc. Natl. Acad. Sci. USA 2006, 103, 17408-17413.
[26] E. Moulin, V. Zoete, S. Barluenga, M. Karplus, N. Winssinger, J. Am. Chem. Soc. 2005, 127, 6999-7004.
[27] S. Barluenga, C. Wang, J. G. Fontaine, K. Aouadi, K. Beebe, S. Tsutsumi, L. Neckers, N. Winssinger, Angew. Chem. Int. Ed. Engl. 2008, 47, 4432-4435; Angew. Chem. 2008, 120, 4504-4507.
[28] P. Y. Dakas, S. Barluenga, F. Totzke, U. Zirrgiebel, N. Winssinger, Angew. Chem. Int. Ed. Engl. 2007, 46, 6899-6902; Angew. Chem. 2008, 119, 7023-7026.
[29] E. Moulin, S. Barluenga, F. Totzke, N. Winssinger, Chem. Eur. J. 2006, 12, 8819-8834; C. Wang, S. Barluenga, G. K. Koripelly, J. G. Fontaine, R. Chen, J. C. Yu, X. Shen, J. C. Chabala, J. V. Heck, A. Rubenstein, N. Winssinger, Bioorg. Med. Chem. Lett. 2009, 19, 3836-3840.
[30] N. Proisy, S. Y. Sharp, K. Boxall, S. Connelly, S. M. Roe, C. Prodromou, A. M. Slawin, L. H. Pearl, P. Workman, C. J. Moody, Chem. Biol. 2006, 13, 1203-1215.
[31] Y. Ikuina, N. Amishiro, M. Miyata, H. Narumi, H. Ogawa, T. Akiyama, Y. Shiotsu, S. Akinaga, C. Murakata, J. Med. Chem. 2003, 46, 2534-2541.
[32] S. Soga, L. M. Neckers, T. W. Schulte, Y. Shiotsu, K. Akasaka, H. Narumi, T. Agatsuma, Y. Ikuina, C. Murakata, T. Tamaoki, S. Akinaga, Cancer Res. 1999, 59, 2931-2938.
[33] M. Scholl, S. Ding, C. W. Lee, R. H. Grubbs, Org. Lett. 1999, 1, 953-956.
[34] J. Kim, S. Felts, L. Llauger, H. He, H. Huezo, N. Rosen, G. Chiosis, J. Biomol. Screen. 2004, 9, 375-381.
[35] W. Xu, E. Mimnaugh, M. F. Rosser, C. Nicchitta, M. Marcu, Y. Yarden, L. Neckers, J. Biol. Chem. 2001, 276, 3702-3708.
[36] S. M. Roe, C. Prodromou, R. O'Brien, J. E. Ladbury, P. W. Piper, L. H. Pearl, J. Med. Chem. 1999, 42, 260-266.
[37] L. Wright, X. Barril, B. Dymock, L. Sheridan, A. Surgenor, M. Beswick, M. Drysdale, A. Collier, A. Massey, N. Davies, A. Fink, C. Fromont, W. Aherne, K. Boxall, S. Sharp, P. Workman, R. E. Hubbard, Chem. Biol. 2004, 11, 775-785.
[38] K. L. Soldano, A. Jivan, C. V. Nicchitta, D. T. Gewirth, J. Biol. Chem. 2003, 278, 48330-48338.
[39] M. Spichty, A. Taly, F. Hagn, H. Kessler, S. Barluenga, N. Winssinger, M. Karplus, Biophys. Chem. 2009, 143, 111-123.
[40] T. E. Barta, J. M. Veal, J. W. Rice, J. M. Partridge, R. P. Fadden, W. Ma, M. Jenks, L. Geng, G. J. Hanson, K. H. Huang, A. F. Barabasz, B. E. Foley, J. Otto, S. E. Hall, Bioorg. Med. Chem. Lett. 2008, 18, 3517-3521.
[41] K. Richter, S. Moser, F. Hagn, R. Friedrich, O. Hainzl, M. Heller, S. Schlee, H. Kessler, J. Reinstein, J. Buchner, J. Biol. Chem. 2006, 281, 11301-11311.

All publications and patent applications as mentioned herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I), or a pharmaceutically acceptable salt, solvate, and/or prodrug thereof: 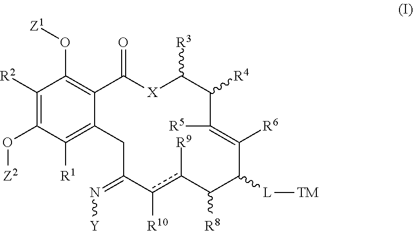
wherein:
X is O, S or NR;
Y is —OR, —O—(CH₂)ₘCOOR, —O—(CH₂)ₘCON(R)₂, —N(R)₂, —N(R)SOR or —N(R)SO₂R, wherein the groups bound to the nitrogen atom may be in Z- or E-configuration;
Z¹ and Z² are independently hydrogen or —(CH₂)—O—R^{Z};
R^{Z} is optionally substituted alkyl;
R¹ and R² are independently hydrogen, halogen, OR, N(R)₂, SR, azido, nitro, cyano, aliphatic, aryl, alkylaryl, arylalkyl, heterocyclyl, heteroaryl, —S(O)R, —S(O)₂R, —SO₂N(R)₂, —N(R)SO₂R, —N(CO)R, —N(CO)N(R)₂, —N(CO)OR, —O(CO)R, —(CO)R, —(CO)OR, —(CO)N(R)₂, —O(CO)OR, or —O(CO)N(R)₂;
R³, R⁴, R⁵, R⁶, R⁸, R⁹ and R¹⁰ are independently hydrogen, halogen, azido, nitro, cyano, aliphatic, alkylaryl, aralkyl, aryl, heteroalkyl, alkylheteroaryl, heterocyclyl, heteroaryl, OR, N(R)₂, SR, —O(CH₂)ₘN(R)C(O)(CH₂)ₚR, —O(CH₂)ₘOC(O)(CH₂)ₚR, —O(CH₂)ₘC(O)(CH₂)ₚN(R)₂, —O(CH₂)ₘC(O)(CH₂)ₚOR, —O(CH₂)ₘN(R)C(O)(CH₂)ₚOR, —O(CH₂)ₘN(R)C(O)(CH₂)ₚN(R)₂, —O(CH₂)ₘOC(O)(CH₂)ₚOR, —O(CH₂)ₘOC(O)(CH₂)ₚN(R)₂, —NR(CH₂)ₘN(R)C(O)(CH₂)ₚR, —NR(CH₂)ₘOC(O)(CH₂)ₚR, —NR(CH₂)ₘC(O)(CH₂)ₚN(R)₂, —NR(CH₂)ₘC(O)(CH₂)ₚOR, —NR(CH₂)ₘN(R)C(O)(CH₂)ₚOR, —NR(CH₂)ₘN(R)C(O)(CH₂)ₚN(R)₂, —NR(CH₂)ₘOC(O)(CH₂)ₚOR, —NR(CH₂)ₘOC(O)(CH₂)ₚN(R)₂, —(CH₂)ₘN(R)C(O)CH₂)ₚR, —(CH₂)ₘOC(O)(CH₂)ₚR, —(CH₂)ₘC(O)(CH₂)ₚN(R)₂, —(CH₂)ₘC(O)(CH₂)ₚOR, —(CH₂)ₘN(R)C(O)(CH₂)ₚOR, —(CH₂)ₘN(R)C(O)(CH₂)ₚN(R)₂, —(CH₂)ₘOC(O)(CH₂)ₚOR, —(CH₂)ₘOC(O)(CH₂)ₚN(R)₂, —(CH₂)ₘN₃, —O(CH₂)ₘN₃—CH₂)ₘN(R)₂, —(CH₂)ₘOR, —(CH₂)ₘS(O)(CH₂)ₚR, —(CH₂)ₘS(O)₂(CH₂)ₚR, —(CH₂)ₘSO₂(CH₂)ₚN(R)₂, or —(CH₂)ₘN(R)SO₂(CH₂)ₚR; and
each R is independently hydrogen, aliphatic, amino, azido, cyano, nitro, alkylamino, dialkylamino, OH, alkoxy, carbonylamino, aminocarbonyl, alkoxycarbonyl, carbonyloxy, carboxy, acyl, aryl, alkaryl, arylalkyl including benzyl, heteroalkyl, heteroaryl, heterocyclyl, or a protecting group; or two R on the same nitrogen are taken together with the nitrogen to form a 5 to 8 membered heterocyclic or heteroaryl ring; wherein where a group contains more than one R substituent; wherein R is optionally substituted, and each R can be the same or different;
m and p are independently 0, 1, 2, 3, 4 or 5;
the dashed lines between the carbon atoms bearing R⁹ and R¹⁰ indicate either a single or a double bond, where the valence requirements are fulfilled by additional hydrogen atoms; and
L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and
TM is a targeting moiety that specifically binds with a biological situs under physiological conditions; or alternatively, L-TM is a group an oxygen- or nitrogen-based functional group; and
with the proviso that structural Formula (I) does not include the compounds listed in Table X.

2. The compound of claim 1, wherein X is O or NR.

3. The compound of claim 1, wherein Y is —OR, —O—(CH₂)ₘCOOR or —O—(CH₂)ₘCON(R)₂.

4. The compound of claim 1, wherein R¹ and R² are independently hydrogen, halogen, or lower alkyl.

5. The compound of claim 4, wherein
R¹ is hydrogen, halogen, or lower alkyl; and
R² is hydrogen.

6. The compound of claim 1, wherein R^{Z} is lower alkyl, alkoxy-substituted lower alkyl, or aryl-substituted lower alkyl.

7. The compound of claim 6, wherein R^{Z} is methyl, ethyl, isopropyl, n-propyl, n-butyl, isobutyl, t-butyl, methoxy-ethyl, methoxy-methyl, chloromethyl, or benzyl.

8. The compound of claim 1, wherein L-TM is an oxygen or nitrogen-based functional group.

9. The compound of claim 1, wherein the compound has the formula (II): 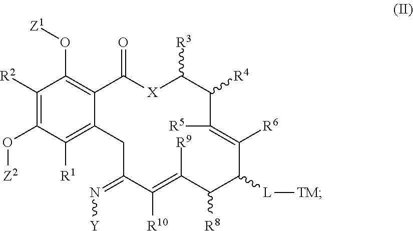
wherein,
X is O, S or NR;
Y is —OR, —O—(CH₂)ₘCOOR, —O—(CH₂)ₘCON(R)₂, —N(R)₂, —N(R)SOR or —N(R)SO₂R, wherein the groups bound to the nitrogen atom may be in Z- or E-configuration;
Z¹ and Z² are independently hydrogen or —(CH₂)—O—R^{Z};
R^{Z} is optionally substituted alkyl;
R¹ and R² are independently hydrogen, halogen, or alkyl;
R³, R⁴, R⁵, R⁶, R⁸, R⁹ and R¹⁰ are independently hydrogen, halogen, or alkyl;
L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and
TM is a targeting moiety that specifically binds with a biological situs under physiological conditions; or alternatively, L-TM is a group an oxygen- or nitrogen-based functional group;

10. The compound of claim 9, wherein the compound has the formula (IIIa): 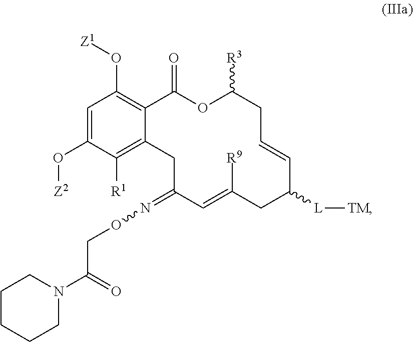
wherein,
Z¹ and Z² are —(CH₂)—O—R^{Z};
R^{Z} is optionally substituted alkyl;
R¹ is H, halogen, or lower alkyl;
R³ and R⁹ are independently H or lower alkyl; and
L-TM is an oxygen-based functional group.

11. The compound of claim 9, wherein the compound has the formula (IIIb): 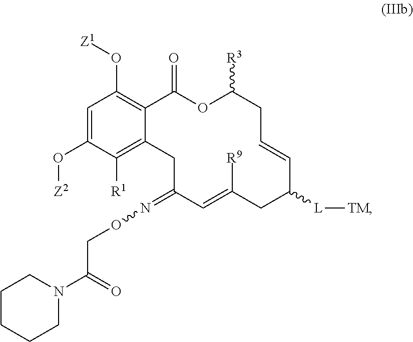
wherein,
and Z² are —(CH₂)—O—R^{Z};
R^{Z} is hydrogen or optionally substituted alkyl;
R¹ is H, halogen, or lower alkyl;
R³ and R⁹ are independently H or lower alkyl; and
L-TM is a nitrogen-based functional group.

12. The compound of claim 9, wherein the compound has the formula (IIIc): 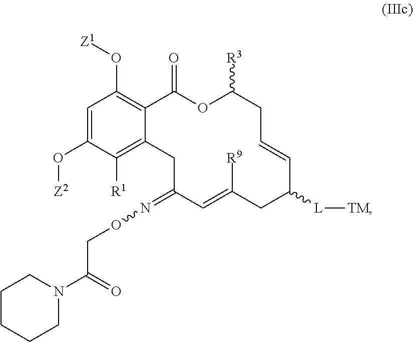
wherein,
Z¹ and Z² are —(CH₂)—O—R^{Z};
R^{Z} is hydrogen or optionally substituted alkyl;
R¹ is H, halogen, or lower alkyl;
R³ and R⁹ are independently H or lower alkyl; and
L is a linkage moiety selected from the group consisting of —O—, —N(R)—, —S—, —C(═O)—, —O—C(═O)—, —C(═O)—O—, —N(R)—C(═O)—, —C(═O)—N(R)—, —O—C(═O)—O—, —O—C(═O)—N(R)—, —N(R)—C(═O)—O—, —N(R)—C(═O)—N(R)—, —C(═O)—O—C(═O)—, —C(═O)—N(R)—C(═O)—, —C(═O)—C(═O)—, —N(R)—N(R)—, —C(═N—NR₂)—, —N(R)—C(═N—NR₂)—, —C(═N—NR₂)—N(R)—, —N(R)—C(═N—NR₂)—N(R)—, —C(═NR)—, —N(R)—C(═NR)—, —C(═NR)—N(R)—, —N(R)—C(═NR)—N(R)—, —C(═S)—, —O—C(═S)—, —C(═S)—O—, —N(R)—C(═S)—, —C(═S)—N(R)—, —O—C(═S)—O—, —O—C(═S)—N(R)—, —N(R)—C(═S)—O—, and —N(R)—C(═S)—N(R)—; and
TM is a targeting moiety that specifically binds with a biological situs under physiological conditions.

13. A compound selected from the group consisting of 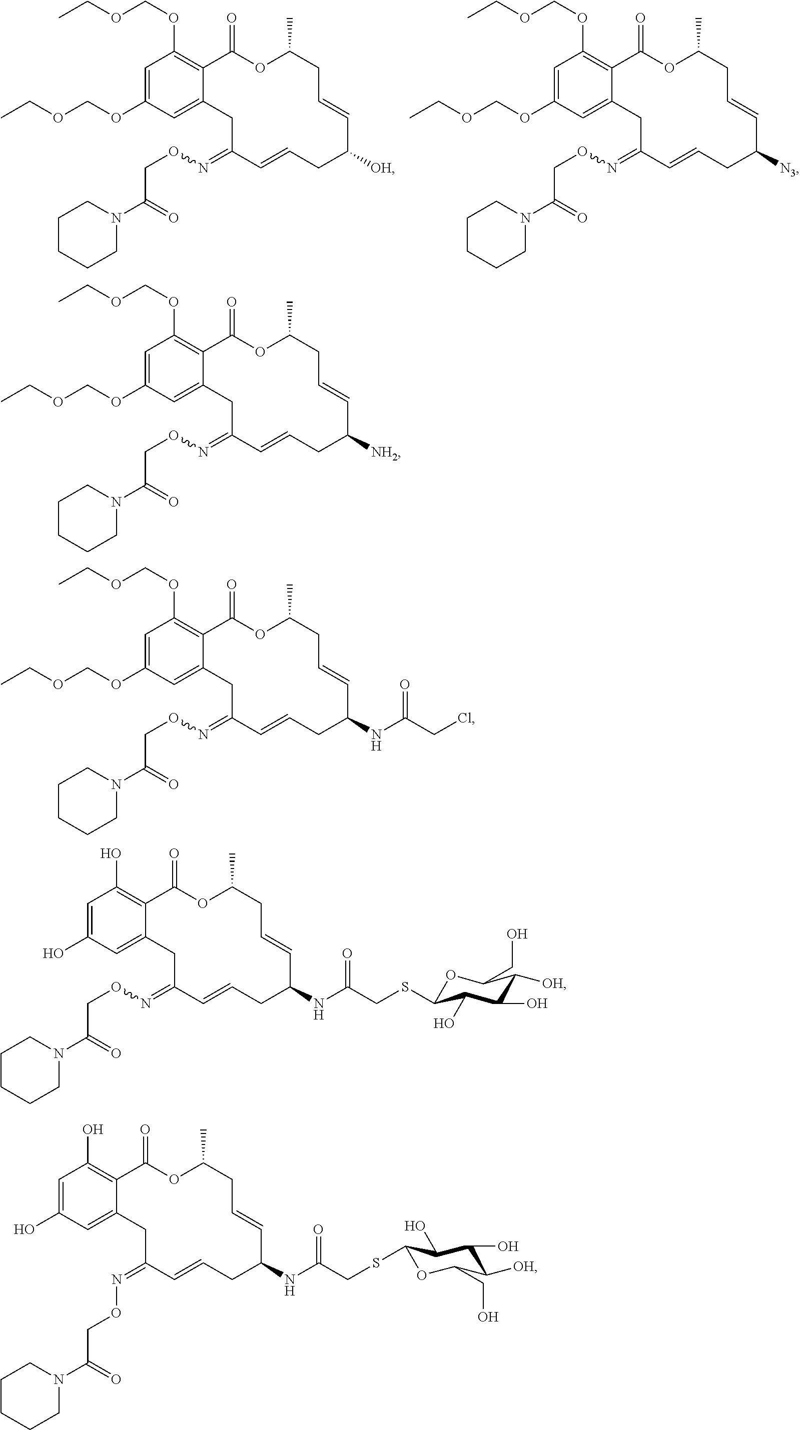 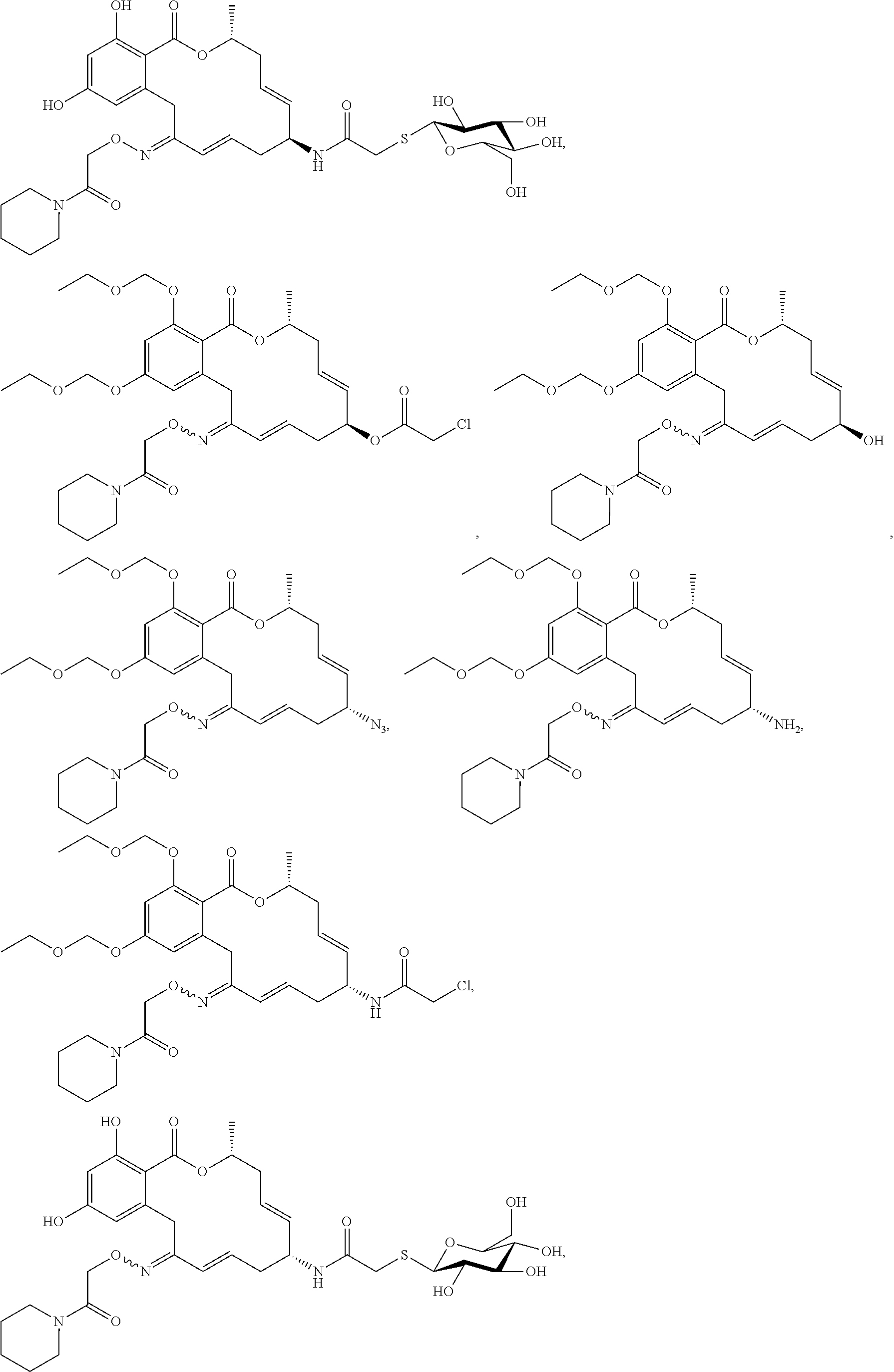 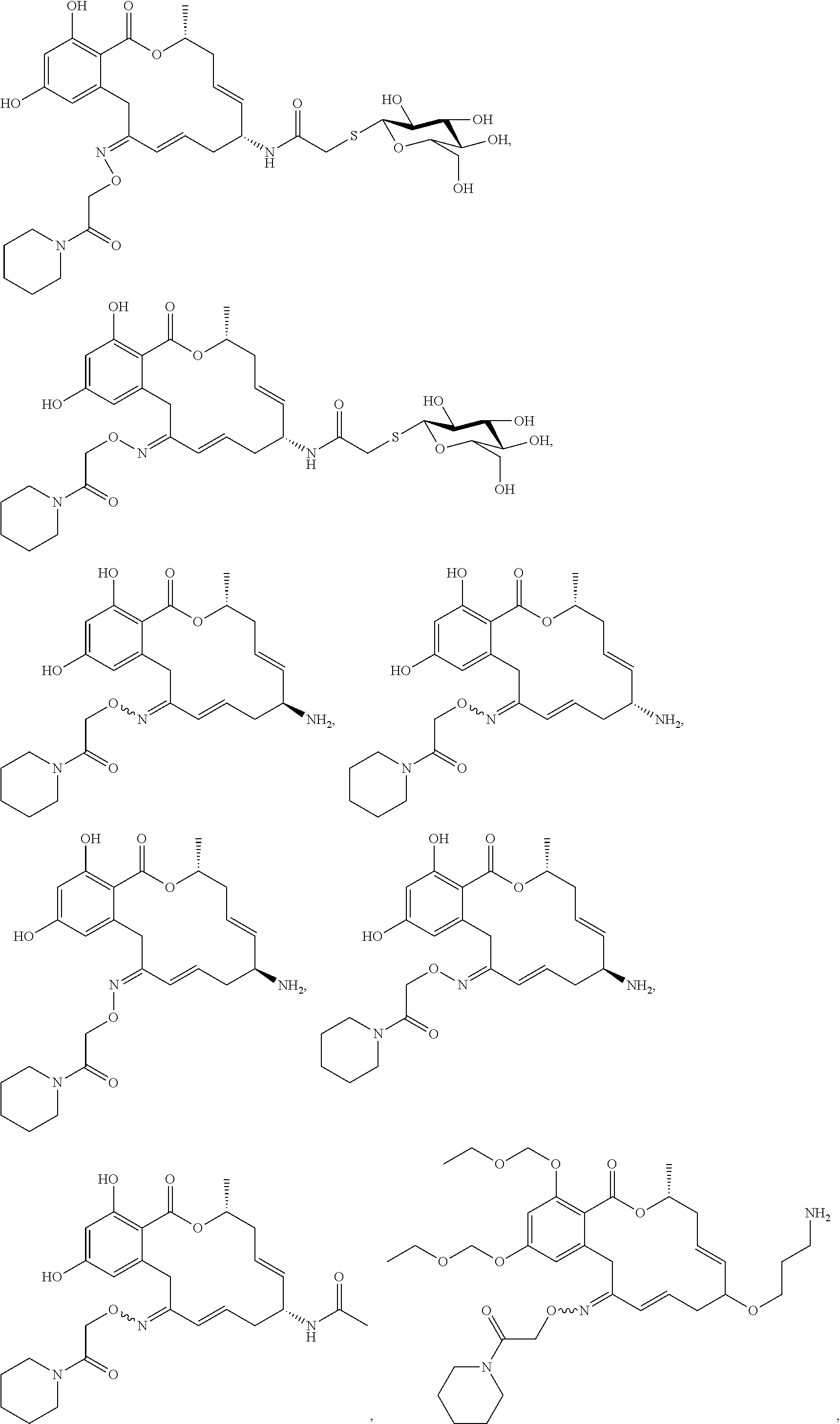 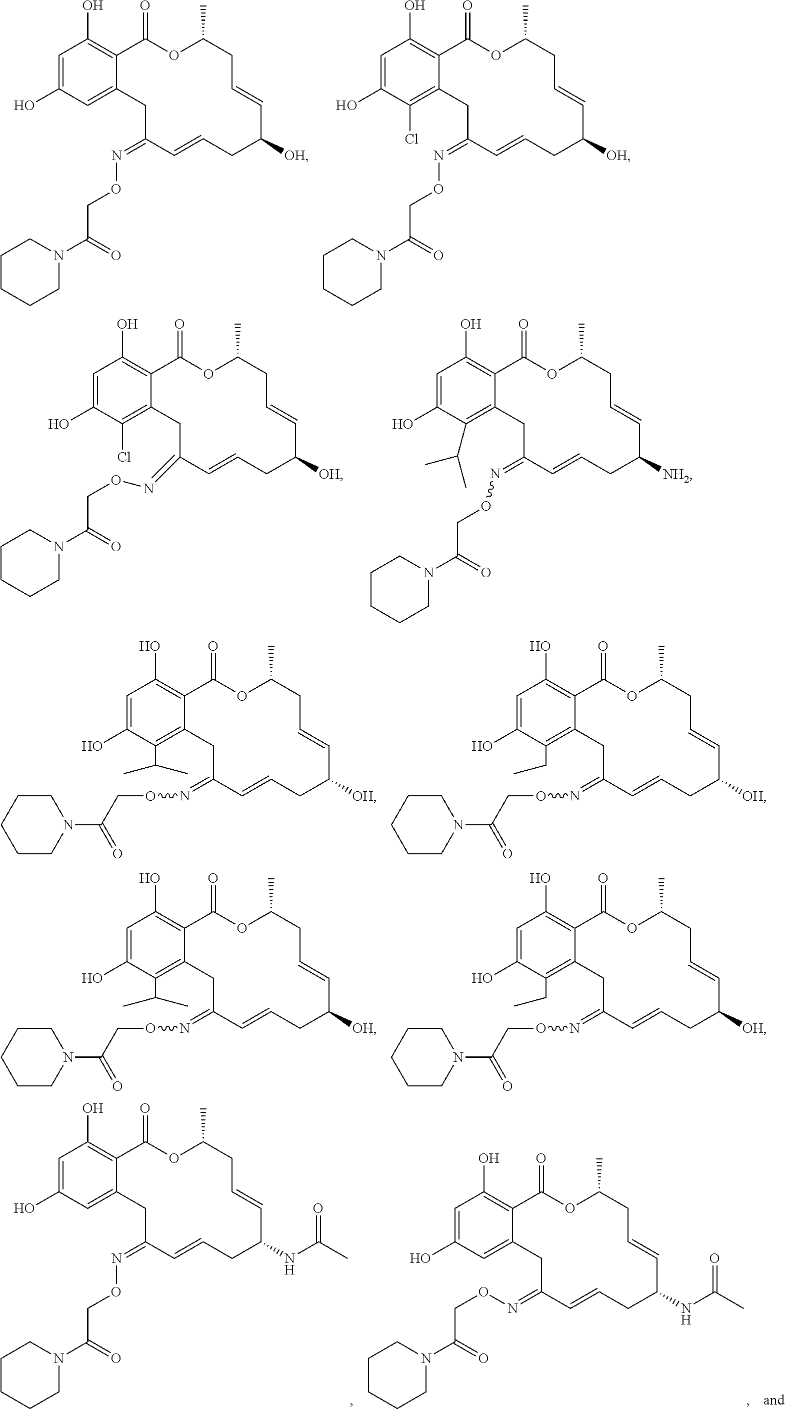 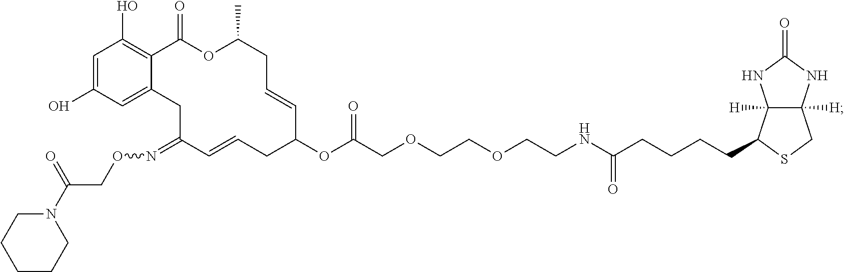
or a pharmaceutically acceptable salt, solvate, and/or prodrug thereof.

14. A compound selected from the group consisting of the compounds exemplified by Scheme 2 as described in the specification, or a pharmaceutically acceptable salt, solvate, and/or prodrug thereof.

15. A pharmaceutical composition comprising a compound of claim 1 and a pharmaceutically acceptable carrier.

16. A method of treating a patient with a disease comprising administering to the patient with the disease an effective amount of a compound of claim 1, wherein the disease is mediated by kinases and Heat Shock Protein 90 (HSP90).

17. The method of claim 16, wherein the disease is an autoimmune disease, inflammatory disease, neurological or neurodegenerative disease, cancer, cardiovascular disease, allergy, asthma, or a hormone-related disease.

18. The method of claim 17, wherein the cancer is a solid tumor, blood borne tumor, breast, ovary, cervix, prostate, testis, genitourinary tract, esophagus, larynx, glioblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

19. The method of claim 17, wherein the inflammatory disease is excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasia, macular degeneration, corneal graft rejection, neovascular glaucoma or Osler Weber syndrome.
